(19) United States
(12) Patent Application Publication
(10) Pub. No.: US 2021/0220135 A1
(43) Pub. Date: Jul. 22, 2021

(54) Apical Control Of Transvascular Delivery Of Prosthetic Mitral Valve

(71) Applicant: Tendyne Holdings, Inc., St. Paul, MN (US)

(72) Inventors: Igor Kovalsky, Minnetonka, MN (US); Zachary J. Tegels, Minneapolis, MN (US); Robert M. Vidlund, Forest Lake, MN (US); Zach Vidlund, Robbinsdale, MN (US)

(73) Assignee: Tendyne Holdings, Inc., St. Paul, MN (US)

(21) Appl. No.: 17/226,445

(22) Filed: Apr. 9, 2021

Related U.S. Application Data
(62) Division of application No. 15/828,834, filed on Dec. 1, 2017.
Application No. 15/828,834 is a continuation of application No. PCT/US2016/035719, filed on Jun. 3, 2016.
(60) Provisional application No. 62/264,562, filed on Dec. 8, 2015.
(60) Provisional application No. 62/171,329, filed on Jun. 5, 2015.

(51) Int. Cl. A61F 2/24

(52) U.S. Cl. CPC A61F 2/2436; A61F 2/2418; A61F 2002/9665; A61F 2/2433; A61F 2/2457; A61F 2/243

(57) ABSTRACT

Apparatus and methods are described herein for use in the delivery and deployment of a prosthetic mitral valve. In some embodiments, after a prosthetic valve has been at least partially delivered from a delivery sheath into the left atrium of a heart, a valve positioning device can be used to engage with and position the prosthetic valve within the mitral annulus. The valve positioning device can include an alignment member configured to engage with a prosthetic valve such that the alignment member can control the distal, proximal, lateral and/or rotational movement of the prosthetic valve. The prosthetic valve can include a tether that can be inserted or threaded through the alignment member such that the alignment member can be moved along the tether into engagement with the prosthetic valve.

### CROSS-REFERENCE TO RELATED APPLICATIONS

This application is a divisional of U.S. application Ser. No. 15/828,834, filed Dec. 1, 2017, which is a continuation of International PCT Application No. PCT/US2016/035719, entitled “Apical Control of Transvascular Delivery of Prosthetic Mitral Valve,” filed Jun. 3, 2016, which claims priority to and the benefit of U.S. Provisional Application Ser. No. 62/264,562, filed Dec. 8, 2015, and U.S. Provisional Patent Application Ser. No. 62/171,329, filed Jun. 5, 2015, each of the disclosures of which is incorporated herein by reference in its entirety.

International PCT Application No. PCT/US2016/035719 is also related to U.S. Provisional Patent Application Ser. No. 61/935,899, entitled “Transfemoral Delivery of Prosthetic Mitral Valve,” filed Feb. 5, 2014, U.S. Provisional Patent Application No. 62/100,548, entitled “Apparatus and Methods for Transfemoral Delivery of Prosthetic Mitral Valve,” filed Jan. 7, 2015, and International Patent Application No. PCT/US2015/014572, entitled “Apparatus and Methods for Transfemoral Delivery of Prosthetic Mitral Valve,” filed Feb. 5, 2015, and International Patent Application No. PCT/US2016/012305, entitled “Prosthetic Mitral Valves and Apparatus and Methods for Delivery of Same,” filed Jan. 6, 2016, each of the disclosures of which is incorporated herein by reference in its entirety.

### BACKGROUND

Embodiments are described herein that relate to devices and methods for use in the delivery and deployment of prosthetic valves, and particularly to devices and methods for delivering expandable prosthetic mitral valves.

Prosthetic heart valves can pose particular challenges for delivery and deployment within a heart. Valvular heart disease, and specifically, aortic and mitral valve disease is a significant health issue in the United States (US); annually approximately 90,000 valve replacements are conducted in the US. Traditional valve replacement surgery involving the orthotopic replacement of a heart valve is considered an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients. Thus elimination of the extra-corporeal component of the procedure could result in reduction in morbidities and cost of valve replacement therapies could be significantly reduced.

While replacement of the aortic valve in a transcatheter manner is the subject of intense investigation, lesser attention has been focused on the mitral valve. This is in part reflective of the greater level of complexity associated to the native mitral valve apparatus, and thus, a greater level of difficulty with regards to inserting and anchoring the replacement prosthesis. A need exists for delivery devices and methods for transcatheter mitral valve replacements.

### SUMMARY

Apparatus and methods are described herein for use in the delivery and deployment of a prosthetic mitral valve. As described herein, in some embodiments, after a prosthetic valve has been at least partially delivered from a delivery sheath into the left atrium of a heart, a valve positioning device can be used to engage with and position the prosthetic valve within the mitral annulus. The valve positioning device can include an alignment member configured to engage with a prosthetic valve such that the alignment member can control the distal, proximal, and/or rotational movement of the prosthetic valve. The prosthetic valve can include a tether that can be inserted or threaded through the alignment member such that the alignment member can be moved along the tether into engagement with the prosthetic valve.

### BRIEF DESCRIPTION OF THE FIGURES

FIGS. 1-6 are each a cross-sectional illustration of a heart with devices used during various stages in a procedure to transfemorally deliver and deploy a prosthetic mitral valve.

FIGS. 7-9 are front, bottom, and top views of a prosthetic heart valve according to an embodiment.

FIG. 10 is an opened and flattened view of the inner frame of the prosthetic heart valve of FIGS. 7-9, in an unexpanded configuration.

FIGS. 11 and 12 are side and bottom views, respectively, of the inner frame of FIG. 10 in an expanded configuration.

FIG. 13 is an opened and flattened view of the outer frame of the valve of FIGS. 7-9, in an unexpanded configuration.

FIGS. 14 and 15 are side and top views, respectively, of the outer frame of FIG. 13 in an expanded configuration.

FIGS. 16-18 are side, front, and top views of an assembly of the inner frame of FIGS. 10-12 and the outer frame of FIGS. 13-15.

FIG. 19 is a side perspective view of the assembly of the inner frame of FIGS. 10-12 and the outer frame of FIGS. 13-15 shown in a biased expanded configuration.

FIG. 20 is a side perspective view of the assembly of FIG. 19 with the outer frame shown inverted.

FIG. 21 is side view of the assembly of FIG. 20 shown in a collapsed configuration within a lumen of a delivery sheath.

FIG. 22 is a side view of the assembly of FIG. 21 shown in a first partially deployed configuration.

FIG. 23 is a side view of the assembly of FIG. 21 shown in a second partially deployed configuration.

FIG. 24 is a side view of the assembly of FIG. 21 shown in a third partially deployed configuration in which the inverted outer frame is substantially deployed outside of the delivery sheath.

FIG. 25 is a side view of the assembly of FIG. 21 shown in a fourth partially deployed configuration in which the outer frame has reverted and assumed a biased expanded configuration.

FIG. 26 is a side view illustrating a portion of a tether coupled to a portion of a valve leader member, according to an embodiment.

FIG. 27 is a side view of a prosthetic mitral valve in a collapsed configuration within a lumen of a portion of a delivery sheath and a balloon dilator device coupled to the delivery sheath.

FIG. 28 is a cross-sectional illustration of a heart with the delivery sheath and balloon dilator device of FIG. 27 at a stage of a procedure to deliver and deploy the prosthetic mitral valve disposed within the delivery sheath.

FIG. 29 is a cross-sectional illustration of a heart with a portion of a delivery sheath shown after deploying a prosthetic mitral valve with the assistance of a wire assist structure, according to an embodiment.

FIG. 30 is a perspective view of the wire assist structure of FIG. 29 coupled to a portion of a prosthetic mitral valve, according to an embodiment.

FIG. 31 is a perspective view of an assist member coupled to a portion of a prosthetic mitral valve, according to an embodiment.

FIG. 32 is a flowchart illustrating a method of delivering a prosthetic mitral valve via a femoral vein, according to an embodiment.

FIG. 33 is a side view of a portion of an epicardial pad device, according to an embodiment, and shown in a collapsed configuration within a delivery sheath.

FIG. 34 is a side perspective view of the epicardial pad device of FIG. 33 shown in an expanded configuration.

FIG. 35 is a side perspective view of a portion of a heart illustrating purse-string sutures at an apex of the heart prior to securing an epicardial pad device thereto.

FIG. 36 is a side perspective view of the epicardial pad device of FIG. 33 shown in the expanded configuration.

FIG. 37 is a bottom perspective view of a portion of a heart illustrating with the epicardial pad device of FIG. 33 secured thereto.

FIG. 38 is an enlarged side perspective view and FIG. 39 is an enlarged bottom view of a portion A in FIG. 37 illustrating an integrated locking mechanism.

FIG. 40 is a side view of an epicardial pad device, according to another embodiment, and shown in a collapsed configuration.

FIG. 41 is a side perspective view of the epicardial pad device of FIG. 40 shown in an expanded configuration.

FIG. 42 is a side view of the epicardial device of FIG. 40 shown in the expanded configuration and being deployed near an apex of a heart.

FIG. 43 is a side view of an epicardial pad device, according to another embodiment, and shown in an expanded configuration being deployed near a heart.

FIG. 44 is a side view of the epicardial pad device of FIG. 43 shown in a collapsed configuration and deployed on the apex of the heart.

FIGS. 45 and 46 are each a side view of an epicardial pad device, according to another embodiment, and shown being deployed on an apex of a heart.

FIG. 47 is a bottom view of a heart with the epicardial pad of FIGS. 45 and 46 secured to the apex of the heart.

FIGS. 48-51A are each a cross-sectional illustration of a heart with a delivery sheath and stopper tube shown at various stages of a procedure to deliver and deploy a prosthetic mitral valve.

FIG. 51B is a cross-sectional illustration of a heart with the delivery sheath and stopper tube of FIGS. 48-51A being used with a procedural catheter.

FIG. 52A is a cross-sectional illustration of a heart with a delivery sheath and valve positioning device shown during a procedure to deliver and deploy a prosthetic mitral valve.

FIG. 52B is cross-sectional illustration of a heart with a delivery sheath and valve positioning device shown during a procedure to deliver and deploy a prosthetic mitral valve being used with a procedural catheter.

FIG. 53 is a schematic illustration of a side view of a prosthetic valve positioning device, according to an embodiment, shown in a first position.

FIG. 54 is a schematic illustration of a side view of the prosthetic valve positioning device of FIG. 53, shown in a second position.

FIG. 55 is a schematic illustration of a side view of a prosthetic valve positioning device, according to an embodiment, shown in a first position.

FIG. 56 is a schematic illustration of a side view of the prosthetic valve positioning device of FIG. 55, shown in a second position.

FIG. 57 is a schematic illustration of a side view of a prosthetic valve positioning device, according to an embodiment, shown in a first position.

FIG. 58 is a schematic illustration of a side view of the prosthetic valve positioning device of FIG. 57, shown in a second position.

FIG. 59 is a schematic illustration of a side view of the prosthetic valve positioning device of FIG. 57, shown in a third position.

FIG. 60 is a cross-sectional illustration of a heart with a delivery sheath and valve positioning device according to another embodiment, shown during a procedure to deliver and deploy a prosthetic mitral valve.

FIG. 61 is a schematic illustration of a side view of a prosthetic valve positioning device, according to an embodiment, shown in a first position.

FIG. 62 is a schematic illustration of a side view of the prosthetic valve positioning device of FIG. 61, shown in a second position.

FIG. 63 is a perspective view of an alignment member of the prosthetic valve positioning device of FIG. 61, shown in a deployed configuration.

FIG. 64 is side view of a prosthetic valve and a valve positioning device, according to another embodiment shown with the valve positioning device in a first position.

FIGS. 65-68 are each a side view of a portion of the valve and valve positioning device of FIG. 64 shown in a second position, a third position, a fourth position, and a fifth position, respectively.

FIGS. 69-74 are each a cross-sectional illustration of a heart with devices used during various stages in a procedure to transfemorally deliver and deploy a prosthetic mitral valve.

FIG. 75 is a flowchart illustrating a method of delivering and deploying a prosthetic mitral valve within a heart.

### DETAILED DESCRIPTION

Apparatus and methods are described herein for use in the delivery and deployment of a prosthetic mitral valve into a heart. As described herein, in some embodiments, after a prosthetic valve has been at least partially delivered from a delivery sheath into the left atrium of a heart, a valve positioning device can be used to engage with and position the prosthetic valve within the mitral annulus. The valve positioning device can include an alignment member configured to engage with a prosthetic valve such that the alignment member can control the distal, proximal, and/or rotational movement of the prosthetic valve. The prosthetic valve can include a tether that can be inserted or threaded through the alignment member such that the alignment member can be moved along the tether into engagement with the prosthetic valve. In some embodiments, maintaining the tether taut while applying a distal force to the alignment member can maintain the engagement between the alignment member and the prosthetic valve. In some embodiments, the prosthetic valve includes engagement portions having particular shapes to increase the secure engagement between the valve positioning device and the prosthetic valve. In some embodiments, the alignment member is configured to maintain controlled engagement with the prosthetic valve even in the absence of a taut tether.

The valve positioning devices described herein can assist in radially realigning a prosthetic valve or other implant while maintaining a low device profile. In some embodiments, the portion of the valve positioning device inserted into the heart can have a diameter or puncture size of, for example, 4 to 8 F. In some embodiments, the valve positioning devices described herein can provide radial torque to align a prosthetic valve. Additionally, the valve positioning devices can move the prosthetic valve atrially or retract the prosthetic valve ventricularly. In some embodiments, the valve positioning devices can partially collapse the prosthetic valve to allow a portion of the implant to be moved ventricularly and deployed below the mitral annulus. The valve positioning devices can be inserted percutaneously through the apex of the heart using a tether as a rail. The valve positioning devices can then be used to partially collapse a prosthetic valve, move it towards the left atrium or left ventricle, radially reorient it, and/or move it into the annulus and allow it to redeploy in the correct orientation.

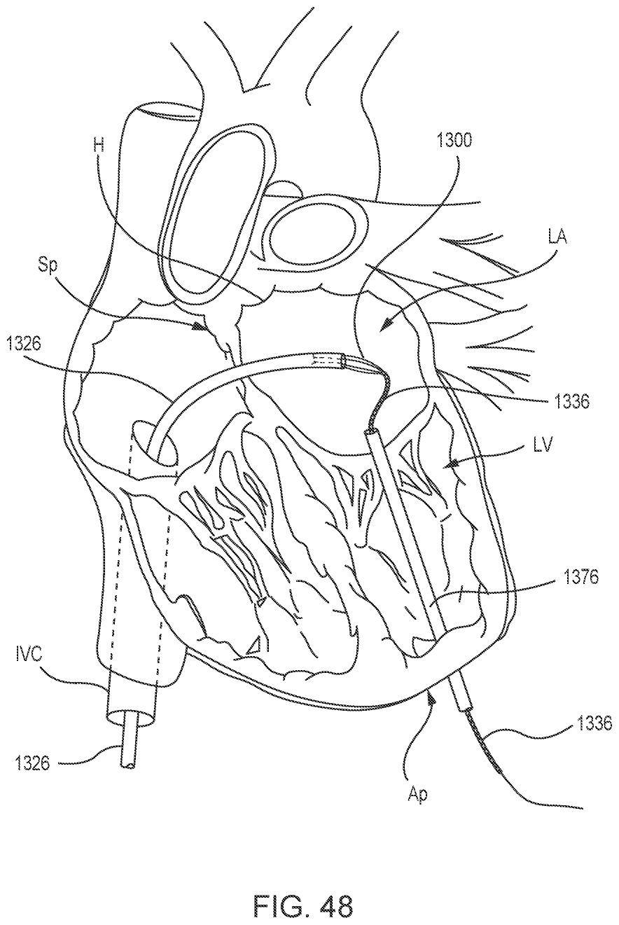

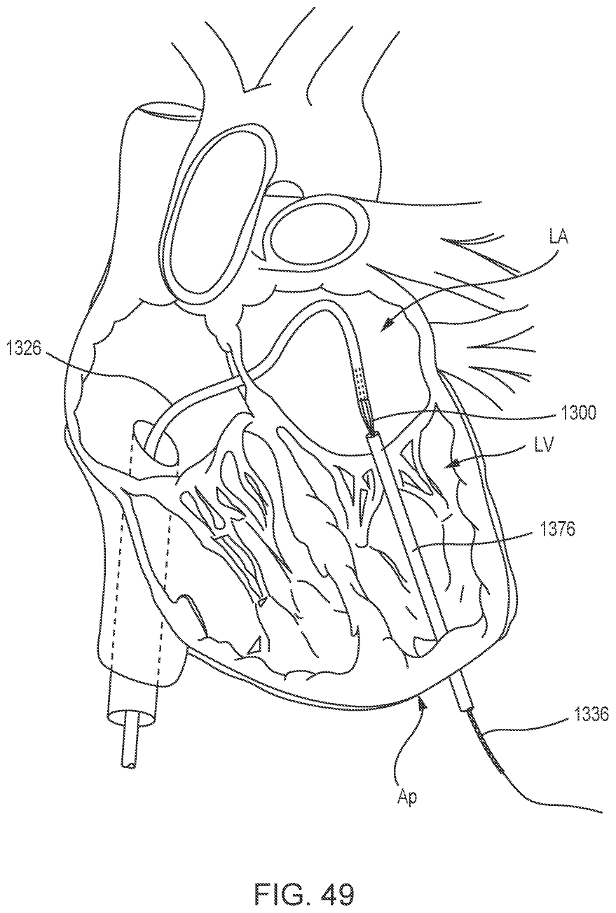

In some embodiments, the valve positioning devices described herein include an expandable Nitinol® braid used to partially recapture the prosthetic valve. In other embodiments, the valve positioning devices include a gooseneck snare used to partially collapse the prosthetic valve. In some embodiments, the valve positioning device is a multiple profile device with a moveable support to keep the smallest profile of the valve positioning device in the ventricle of the heart. In some embodiments, the valve positioning device is a multiple profile device that can provide the greatest amount of torque to the prosthetic valve by maximizing torque transfer through reducing the portion of the low profile portion of the device that extends outside of the apex of the heart. In other words, although a smaller diameter portion of the device is needed to be inserted into the heart, the device can include a larger profile portion of the device that extends outside of the heart that can provide strength to the device and improve torqueability. In other embodiments, the valve positioning device can slightly dimple or compress the apex of the heart to further increase the amount of torque applied to the prosthetic valve and minimize the portion of the low profile portion of the device that extends outside of the apex. Further details of specific embodiments of a valve positioning device are described below with reference to FIGS. 48-63.

As described herein, in some embodiments, a method includes inverting an outer frame of a prosthetic mitral valve when in a biased expanded configuration. The prosthetic mitral valve is formed with a shape-memory material. After inverting the outer frame, the prosthetic mitral valve is inserted into a lumen of a delivery sheath such that the mitral valve is moved to a collapsed configuration. The delivery sheath is inserted into a femoral vein of a patient and moved through the femoral vein and a septum of a heart of the patient until a distal end portion of the delivery sheath is disposed in the left atrium of the heart. The prosthetic mitral valve is moved distally out of the delivery sheath such that the inverted outer frame reverts and the prosthetic mitral valve assumes its biased expanded configuration. The prosthetic mitral valve is then positioned within a mitral annulus of the heart.

In some embodiments, a method include disposing a prosthetic heart valve at least partially within a left atrium of a heart such that a tether coupled to the prosthetic heart valve extends through the left ventricle and outside of the apex of the heart. A proximal end of the tether is threaded through a lumen defined by an alignment member. While moving along the tether, a distal end portion of the alignment member is inserted into the left ventricle, through the native mitral annulus and into the left atrium of the heart. A first portion of the prosthetic heart valve is engaged with a distal end portion of the alignment member. With the prosthetic heart valve engaged with the alignment member, the prosthetic heart valve is moved to a desired position within a native mitral annulus of the heart by collectively moving the alignment member and the prosthetic heart valve. For example, in some embodiments, moving the prosthetic heart valve to a desired position within a native mitral annulus of the heart includes rotating the prosthetic heart valve about a central axis of the prosthetic heart valve and/or laterally moving an orientation of the prosthetic heart valve relative to the native mitral annulus.

In some embodiments, an apparatus includes a handle assembly and an elongate member that defines a lumen and is operatively coupled to the handle assembly. The handle assembly includes an actuator configured to move the elongate member proximally and distally relative to the handle assembly. The elongate member includes an engagement portion configured to engage a portion of a prosthetic valve during deployment of the prosthetic valve within a heart when the elongate member is moved distally relative to the handle. The engagement portion is configured to move the portion of the prosthetic valve to at least a partially collapsed configuration when engaged with the prosthetic valve. The elongate member and the prosthetic valve are configured to collectively move together when the first engagement feature is engaged with the second engagement feature such that the prosthetic valve can be moved by manipulation of the handle.

In some embodiments, an apparatus includes a handle assembly and an elongate member that defines a lumen and is operatively coupled to the handle assembly. The handle assembly includes an actuator configured to move the elongate member proximally and distally relative to the handle assembly. The elongate member includes a first engagement feature configured to matingly engage with and releasably couple to a second engagement feature of a prosthetic valve during deployment of the prosthetic valve within a heart when the elongate member is moved distally relative to the handle. The elongate member and the prosthetic valve are configured to collectively move together when the first engagement feature is engaged with the second engagement feature such that the prosthetic valve can be moved by manipulation of the handle.

Although the embodiments described herein are described in reference to a transfemoral delivery approach, the devices described herein can be used to deliver a prosthetic heart valve to a heart using any suitable delivery approach. For example, the prosthetic valves described herein can be delivered using a transfemoral delivery approach as described, for example, in International Application No. PCT/US15/14572 (the '572 PCT application) and International Application No. PCT/US16/12305 (the '305 PCT application) incorporated by reference above, or via a transatrial approach, such as described in U.S. Provisional Patent Application Ser. No. 62/220,704, entitled “Apparatus and Methods for Transatrial Delivery of Prosthetic Mitral Valve,” filed Sep. 18, 2015 (the '704 application”), which is incorporated herein by reference in its entirety. In such a case, the inverted valve would enter the heart through the atrium similarly to the transfemoral delivery approach. The valve positioning devices described herein can be used to position the inverted valve once it is at least partially deployed in the left atrium. In another example, an inverted valve as described herein could be delivered via a transjugular approach, via the right atrium and through the atrial septum, such as described in U.S. Provisional Patent Application Ser. No. 62/305,678, entitled “Apparatus and Methods for Delivery of Prosthetic Mitral Valve,” filed Mar. 9, 2106 (the '678 application”), the disclosure of which is incorporated herein by reference in its entirety. In such a case, the valve positioning devices described herein can be used similarly to position the inverted valve after the valve has been at least partially deployed in the left atrium. The prosthetic valves described herein can also be delivered apically if desired.

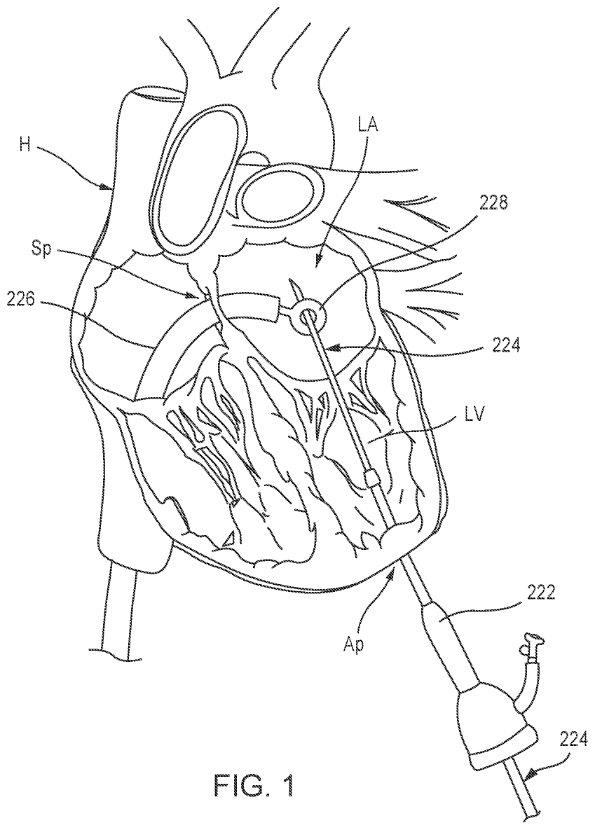

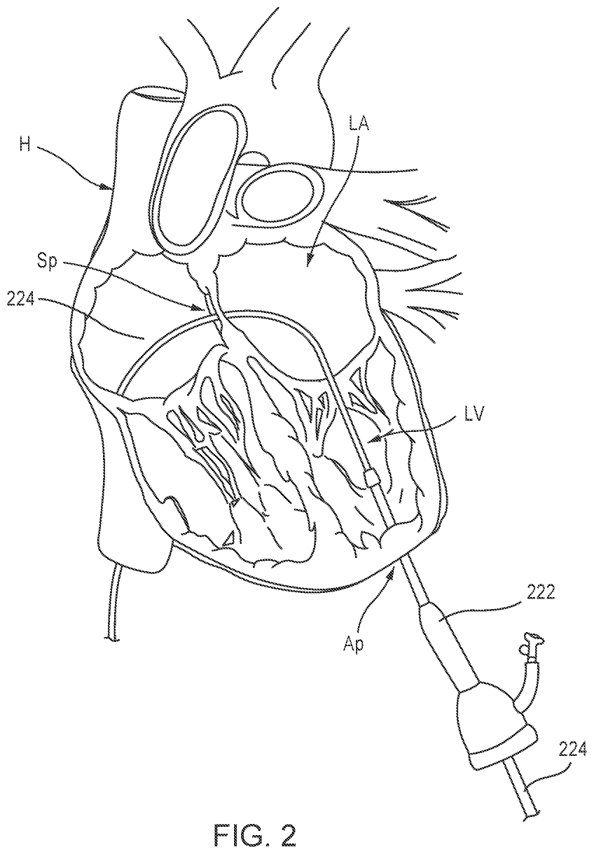

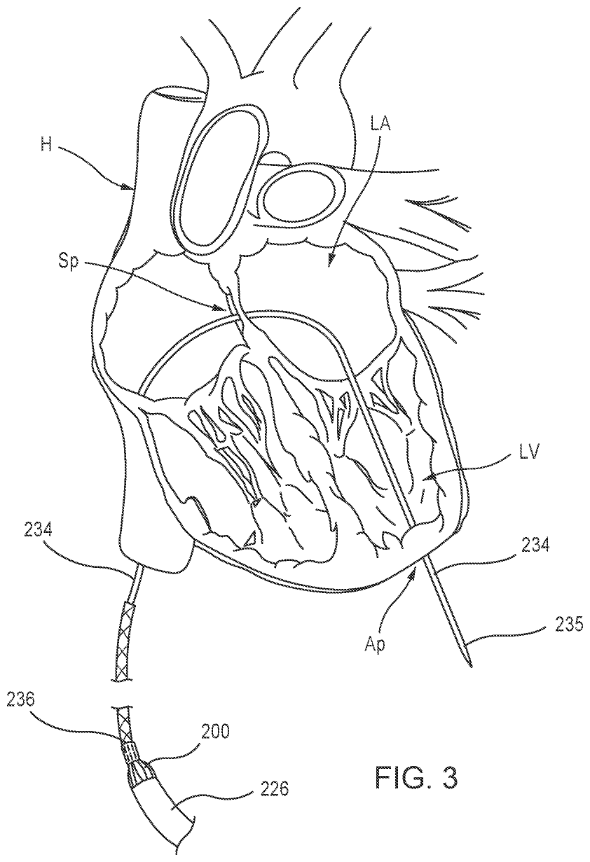

FIGS. 1-6 illustrate a method of delivering a prosthetic mitral valve 200 (shown in FIGS. 3-6) to a left atrium LA of a heart H via introduction through a femoral vein. As shown in FIG. 1, a procedural catheter 222 is inserted through an apical puncture (e.g., a 5F apical puncture) in a ventricular wall at the apex Ap of the heart H. A leader tube 224 is inserted through a lumen (not shown) of the procedural catheter 222 and extended through the left ventricle LV, through a mitral valve gap and into the left atrium LA. A delivery sheath 226 is introduced through a femoral vein puncture and extended through the inferior vena cava, into the right atrium, and then through a transseptal puncture of the septum Sp of the heart H, and into the left atrium LA of the heart H. A snare device 228 is movably disposed within the delivery sheath 226 and used to grab or snare a distal end portion of the leader tube 224, as shown in FIG. 1. The snare device 228 can be used to pull the leader tube 224 through the delivery sheath 226 such that the distal end portion of the leader tube 224 extends outside the femoral vein and a proximal end of the leader tube 224 is disposed through the ventricular wall at the apex Ap of the heart H, as shown in FIG. 2. The leader tube 224 allows for back-loading of the prosthetic mitral valve 200 starting in the femoral vein and exiting the heart H at the apex Ap. Although not shown in FIGS. 1 and 2, the procedural catheter 224 is disposed outside the patient's body, the distal end of the leader tube 224 extends outside the femoral vein and outside the patient's body, and the proximal end of the leader tube 224 extends outside the apex Ap and outside the patient's body. Although the above described snare process describes delivering the leader tube 224 to the left atrium of the heart and then snaring the leader tube 224 using the snare device 228, in alternative embodiments, the leader tube 224 can be delivered to the left ventricle LV and the snare device 228 and delivery sheath 226 can be inserted through the mitral annulus and into the left ventricle LV to grab or snare the leader tube 224 as described above.

After the leader tube 224 has been extended between the apex Ap and the access site to the femoral vein, a valve leader member 234 attached to the prosthetic mitral valve 200 (also referred to as “valve”) can be inserted into the leader tube 224 at the femoral end of the leader tube 224 and extended through the leader tube 224 until the valve leader member 234 exits the leader tube at the apex end of the leader tube 224. After the valve leader member 234 is inserted and extended outside the apex Ap, the leader tube 224 can be removed from the patient. For example, the leader tube 224 can be pulled out through the apex puncture site, or through the femoral vein puncture site. Thus, only the valve leader member 234 remains disposed within the body, as shown in FIG. 3.

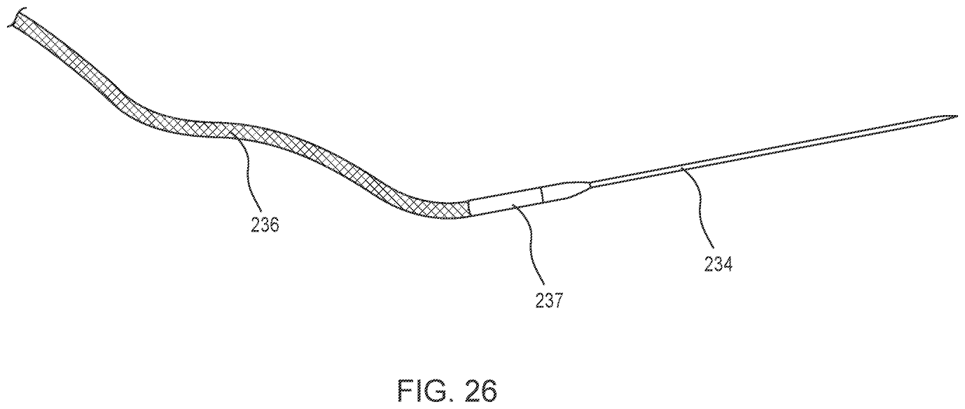

The valve leader member 234 can have a tapered distal end 235 to aid in the insertion and maneuvering of the valve leader member 234 through the leader tube 224. The valve leader member 234 is attached at a proximal end portion 237 to a tether line 236 (also referred to herein as “tether”), which is attached to the valve 200. FIG. 26 illustrates an enlarged view of the attachment of the proximal end portion 237 to tether 236. The tether 236 can be formed, for example, as a braided rope or cord as shown, for example, in FIG. 26.

As shown in FIG. 3, the valve 200 is partially disposed within a lumen of the delivery sheath 226. Although the delivery sheath 226 is used to deliver both the snare device 228 and the valve 200, in other embodiments, a different delivery sheath can be used to deliver the snare device 228 than is used to deliver the valve 200. As shown in FIG. 3, prior to inserting the valve leader member 234 into the leader tube 224, the procedural catheter 222 can be removed. Alternatively, the procedural catheter 222 can be removed after inserting the valve leader member 234.

Also as shown in FIG. 3, in this embodiment, a portion of the valve 200 is allowed to partially deploy outside a distal end of the delivery sheath 226. The partially deployed portion of the valve 200 can be used as a lead-in to the delivery sheath 226 as the valve 200 is inserted through femoral vein. For example, the valve 200 can be formed with a shape-memory material (as described in more detail below) and can have a biased undeformed shape and can be manipulated and/or deformed (e.g., compressed and/or expanded) and, when released, return to its original undeformed shape. In some embodiments, the valve 200 can have a biased expanded or undeformed configuration when deployed within a heart, and can be moved to a collapsed or deformed configuration when placed within the lumen of the delivery sheath 226 for delivery through the femoral vein. The valve can be, for example, a valve constructed the same as or similar to, and function in the same or similar manner as, the prosthetic heart valve 500, described in detail below.

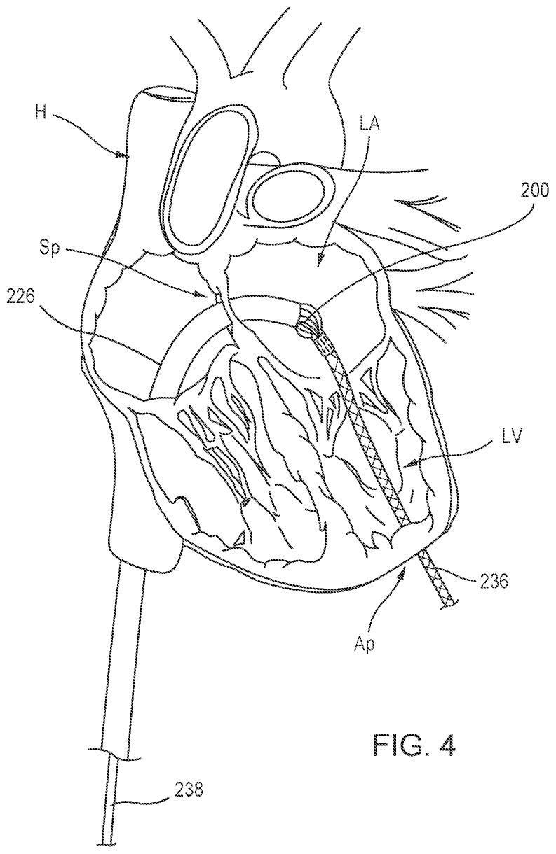

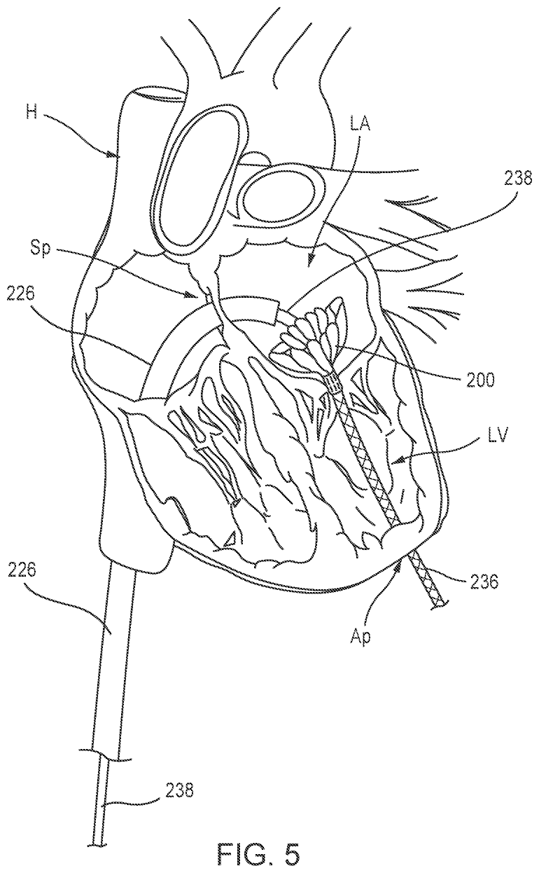

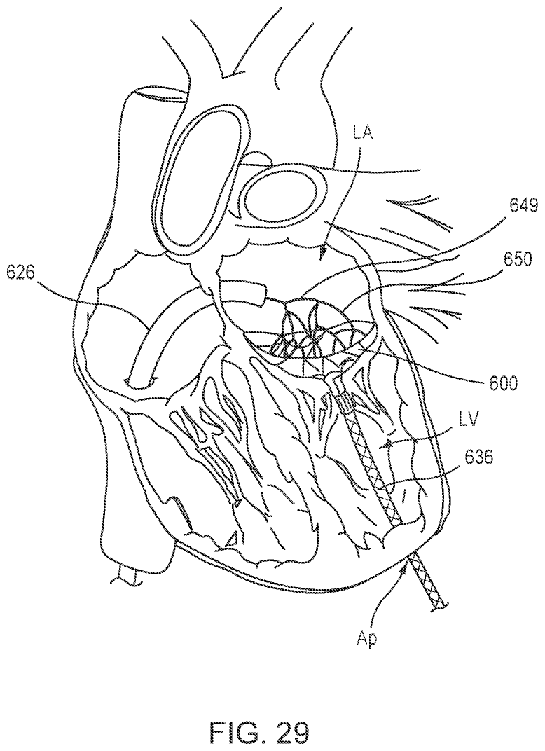

After the valve leader member 234 has been placed in position between the femoral puncture site and the apical puncture site, as described above, the delivery sheath 226 with the valve 200 can be inserted through the femoral puncture site and moved through the femoral vein, through the inferior vena cava, into the right atrium, and then through the septum Sp until a distal end portion of the delivery sheath 226 (with the valve 200) is disposed within the left atrium LA, as shown in FIG. 4. As shown in FIG. 4, the tether 236 extends from the valve 200 through the apical puncture and outside the patient's body. As the delivery sheath 226 is advanced, the tether 236 can optionally be pulled at the apex end to help move the delivery sheath 226, with the valve 200 disposed therein, through the femoral vein, through the septal puncture and into the left atrium LA. The valve 200 can then be fully deployed within the left atrium LA (see, e.g., FIG. 5) by pulling the apex end portion of the tether 236 until the valve 200 is pulled out of the lumen of the delivery sheath 226 and disposed within the left atrium LA. Alternatively, pusher device 238 (see, e.g., FIG. 4) can be inserted within the delivery sheath 226 and used to push the valve 200 outside a distal end of the delivery sheath 226. In yet other embodiments, the pusher device 238 can be used push the valve 200 while the tether 236 is pulled. In other words, the valve 200 can be deployed by pushing the valve 200 with the pusher device 238, by pulling the valve 200 with the tether 236, or both. The pusher 238 can also be used to aid in positioning the valve 200 in a desired radial orientation within the left atrium LA. For example, the pusher device 238 can define an internal lumen (not shown) that can be placed over an inner frame portion of the valve 200 to hold the inner frame portion in a small diameter, which can help enable the valve 200 to be positioned in a desired radial orientation and be seated within the annulus of the mitral valve. Further examples of such a valve assist device are described below with reference to FIGS. 29-31.

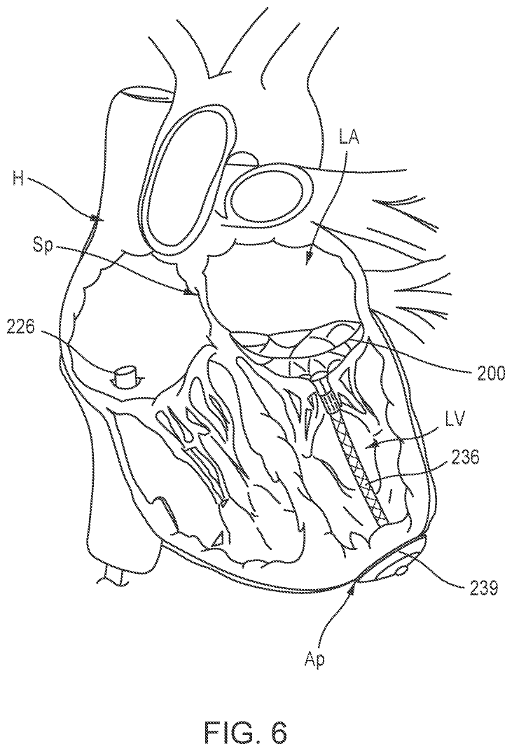

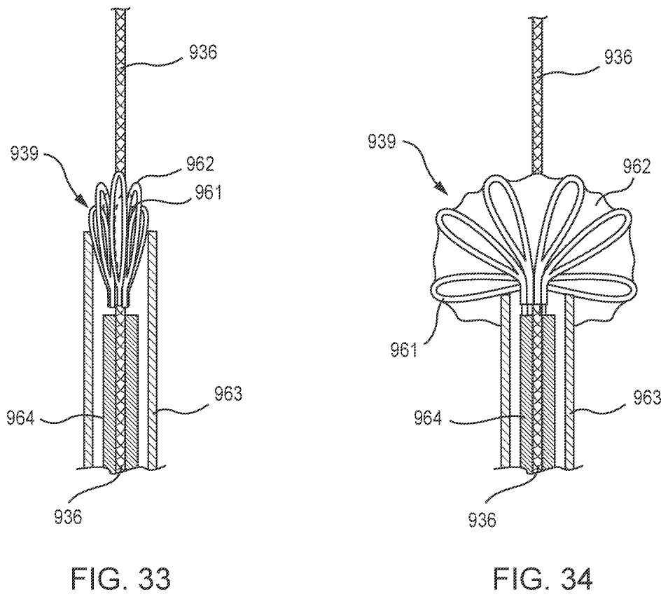

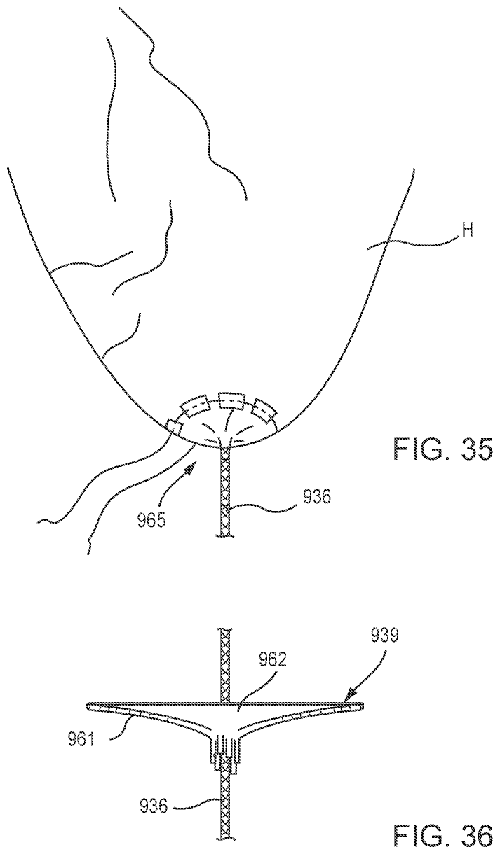

As shown in FIGS. 5 and 6, as the valve 200 is deployed within the left atrium LA, the valve 200 is allowed to assume its biased expanded or deployed configuration. The delivery sheath 226 can then be removed from the patient and the valve 200 can be positioned and tensioned using the tether 236 to obtain the desired or optimal location in the native mitral annulus and minimize perivalvular leaks. An epicardial pad device 239 can be used to secure the tether 236 and valve 200 in position within the mitral annulus as shown in FIG. 6. For example, an epicardial pad device as described in International Patent Application No. PCT/US14/49218 (“the '218 PCT application”), the disclosure of which is incorporated herein by reference in its entirety, can be used. In some embodiments, an expandable epicardial pad can be used to secure the tether and valve in position. Examples embodiments of expandable pads that can be used are described herein with reference to FIGS. 33-47. Such a pad can be smaller in size such that the pad can be delivered to the heart via a small incision and small catheter or delivery sheath. In some embodiments, a positioning device (not shown) can be used to help position the valve 200 and deploy the epicardial pad device. For example, a positioning device as described in the '218 PCT application incorporated by reference above, or devices described in International Patent Application No. PCT/US14/61046, the disclosure of which is incorporated herein by reference in its entirety, can be used. In some embodiments, rather than securing the prosthetic mitral valve with a tether and epicardial pad, the prosthetic mitral valve can be secured with clips or other coupling methods to a portion(s) of the mitral valve apparatus and/or the ventricular wall of the heart. For example, such coupling methods are described in International Patent Application No. PCT/US14/58826 (“the '826 PCT application”), the disclosure of which is incorporated herein by reference in its entirety.

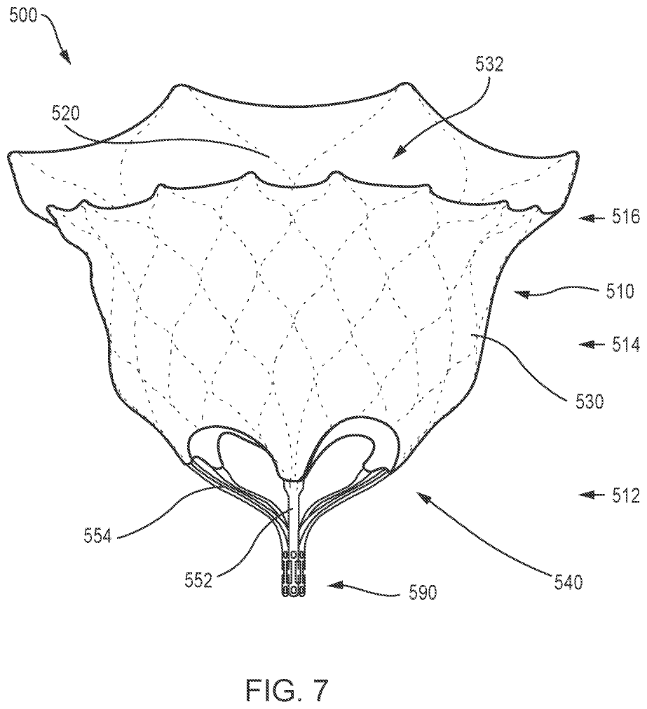

FIGS. 7-9 illustrate an embodiment of a prosthetic heart valve that can be delivered and deployed within a left atrium of a heart using a transfemoral delivery approach as described above. FIGS. 7-9 are front, bottom, and top views, respectively, of a prosthetic heart valve 500 according to an embodiment. Prosthetic heart valve 500 (also referred to herein as “valve”) is designed to replace a damaged or diseased native heart valve such as a mitral valve. Valve 500 includes an outer frame assembly 510 and an inner valve assembly 540 coupled to the outer frame assembly 510.

As shown, outer frame assembly 510 includes an outer frame 520, covered on all or a portion of its outer face with an outer covering 530, and covered on all or a portion of its inner face by an inner covering 532. Outer frame 520 can provide several functions for prosthetic heart valve 500, including serving as the primary structure, as an anchoring mechanism and/or an attachment point for a separate anchoring mechanism to anchor the valve to the native heart valve apparatus, a support to carry inner valve assembly 540, and/or a seal to inhibit paravalvular leakage between prosthetic heart valve 500 and the native heart valve apparatus.

Outer frame 520 is configured to be manipulated and/or deformed (e.g., compressed and/or expanded) and, when released, return to its original (undeformed) shape. To achieve this, outer frame 520 can be formed of materials, such as metals or plastics, that have shape memory properties. With regards to metals, Nitinol® has been found to be especially useful since it can be processed to be austenitic, martensitic or superelastic. Other shape memory alloys, such as Cu—Zn—Al—Ni alloys, and Cu—Al—Ni alloys, may also be used.

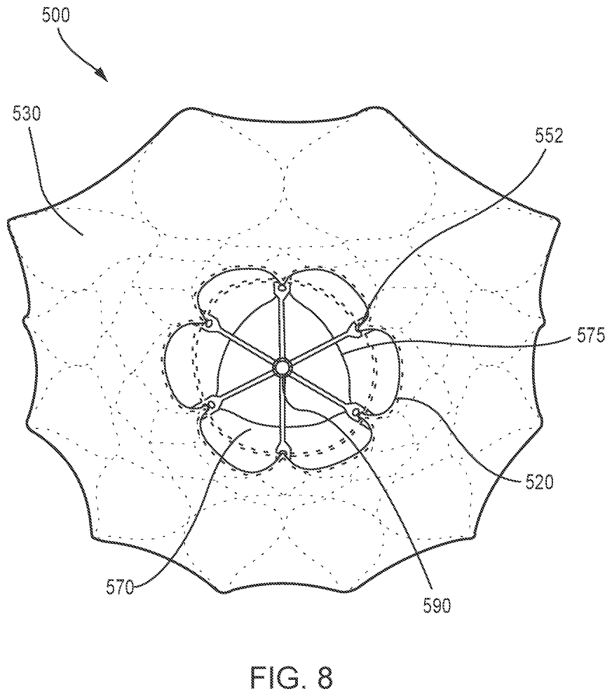

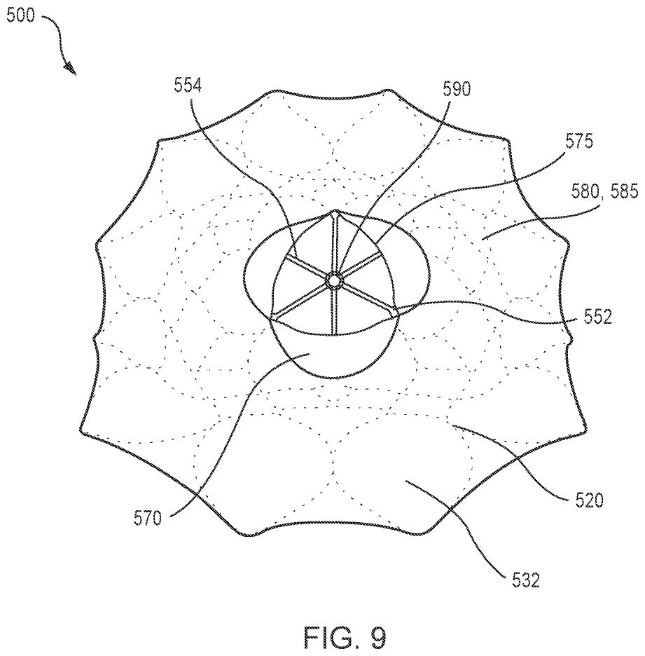

As best shown in FIG. 7, outer frame assembly 510 has an upper end (e.g., at the atrium portion 516), a lower end (e.g., at the ventricle portion 512), and a medial portion (e.g., at the annulus portion 514) therebetween. The medial portion of the outer frame assembly 510 has a perimeter that is configured (e.g., sized, shaped) to fit into an annulus of a native atrioventricular valve. The upper end of the outer frame assembly 510 has a perimeter that is larger than the perimeter of the medial portion. In some embodiments, the perimeter of the upper end of the outer frame assembly 510 has a perimeter that is substantially larger than the perimeter of the medial portion. As shown best in FIG. 9, the upper end and the medial portion of the outer frame assembly 510 has a D-shaped cross-section. In this manner, the outer frame assembly 510 promotes a suitable fit into the annulus of the native atrioventricular valve.

Inner valve assembly 540 includes an inner frame 550, an outer covering 560, and leaflets 570. As shown, the inner valve assembly 540 includes an upper portion having a periphery formed with multiple arches. The inner frame 550 includes six axial posts or frame members that support outer covering 560 and leaflets 570. Leaflets 570 are attached along three of the posts, shown as commissure posts 552 (best illustrated in FIG. 8), and outer covering 560 is attached to the other three posts, 554 (best illustrated in FIG. 8), and optionally to commissure posts 552. Each of outer covering 560 and leaflets 570 are formed of approximately rectangular sheets of material, which are joined together at their upper, or atrium end. The lower, ventricle end of outer covering 560 may be joined to inner covering 532 of outer frame assembly 510, and the lower, ventricle end of leaflets 570 may form free edges 575, though coupled to the lower ends of commissure posts 552.

Although inner valve assembly 540 is shown as having three leaflets, in other embodiments, an inner valve assembly can include any suitable number of leaflets. The leaflets 570 are movable between an open configuration and a closed configuration in which the leaflets 570 coapt, or meet in a sealing abutment.

Outer covering 530 of the outer frame assembly 510 and inner covering 532 of outer frame assembly 510, outer covering 560 of the inner valve assembly 540 and leaflets 570 of the inner valve assembly 540 may be formed of any suitable material, or combination of materials, such as those discussed above. In this embodiment, the inner covering 532 of the outer frame assembly 510, the outer covering 560 of the inner valve assembly 540, and the leaflets 570 of the inner valve assembly 540 are formed, at least in part, of porcine pericardium. Moreover, in this embodiment, the outer covering 530 of the outer frame assembly 510 is formed, at least in part, of polyester.

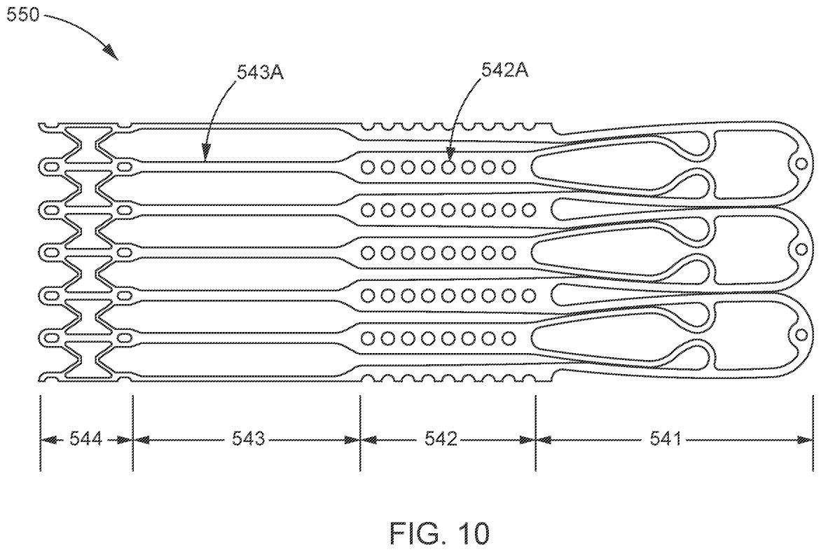

Inner frame 550 is shown in more detail in FIGS. 10-12. Specifically, FIGS. 10-12 show inner frame 550 in an undeformed, initial state (FIG. 10), a side view of the inner frame 550 in a deployed configuration (FIG. 11), and a bottom view of the inner frame 550 in a deployed configuration (FIG. 12), respectively, according to an embodiment.

In this embodiment, inner frame 550 is formed from a laser-cut tube of Nitinol®. Inner frame 550 is illustrated in FIG. 10 in an undeformed, initial state, i.e. as laser-cut, but cut and unrolled into a flat sheet for ease of illustration. Inner frame 550 can be divided into four portions, corresponding to functionally different portions of the inner frame 550 in final form: atrial portion 541, body portion 542, strut portion 543, and tether clamp or connecting portion 544. Strut portion 543 includes six struts, such as strut 543A, which connect body portion 542 to tether clamp portion 544.

Connecting portion 544 includes longitudinal extensions of the struts, connected circumferentially by pairs of opposed, slightly V-shaped connecting members (or “micro-Vs”). Connecting portion 544 is configured to be radially collapsed by application of a compressive force, which causes the micro-Vs to become more deeply V-shaped, with the vertices moving closer together longitudinally and the open ends of the V shapes moving closer together circumferentially. Thus, connecting portion 544 can be configured to compressively clamp or grip one end of a tether, either connecting directly onto a tether line (e.g. braided filament line) or onto an intermediate structure, such as a polymer or metal piece that is in turn firmly fixed to the tether line.

In contrast to connecting portion 544, atrial portion 541 and body portion 542 are configured to be expanded radially. Strut portion 543 forms a longitudinal connection, and radial transition, between the expanded body portion and the compressed connecting portion 544.

Body portion 542 includes six longitudinal posts, such as post 542A. The posts can be used to attach leaflets 570 to inner frame 540, and/or can be used to attach inner assembly 540 to outer assembly 510, such as by connecting inner frame 550 to outer frame 520. In the illustrated embodiment, the posts include openings through which connecting members (such as suture filaments and/or wires) can be passed to couple the posts to other structures.

Inner frame 550 is shown in a fully deformed, i.e. the final, deployed configuration, in side view and bottom view in FIGS. 11 and 12, respectively.

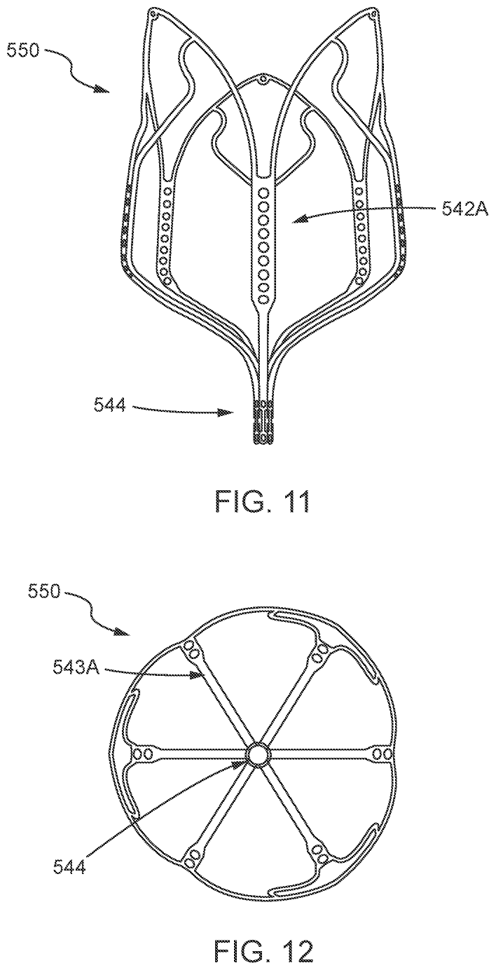

Outer frame 520 of valve 500 is shown in more detail in FIGS. 13-15. In this embodiment, outer frame 520 is also formed from a laser-cut tube of Nitinol®. Outer frame 520 is illustrated in FIG. 13 in an undeformed, initial state, i.e. as laser-cut, but cut and unrolled into a flat sheet for ease of illustration. Outer frame 520 can be divided into a coupling portion 571, a body portion 572, and a cuff portion 573, as shown in FIG. 13. Coupling portion 571 includes multiple openings or apertures, such as 571A, by which outer frame 520 can be coupled to inner frame 550, as discussed in more detail below.

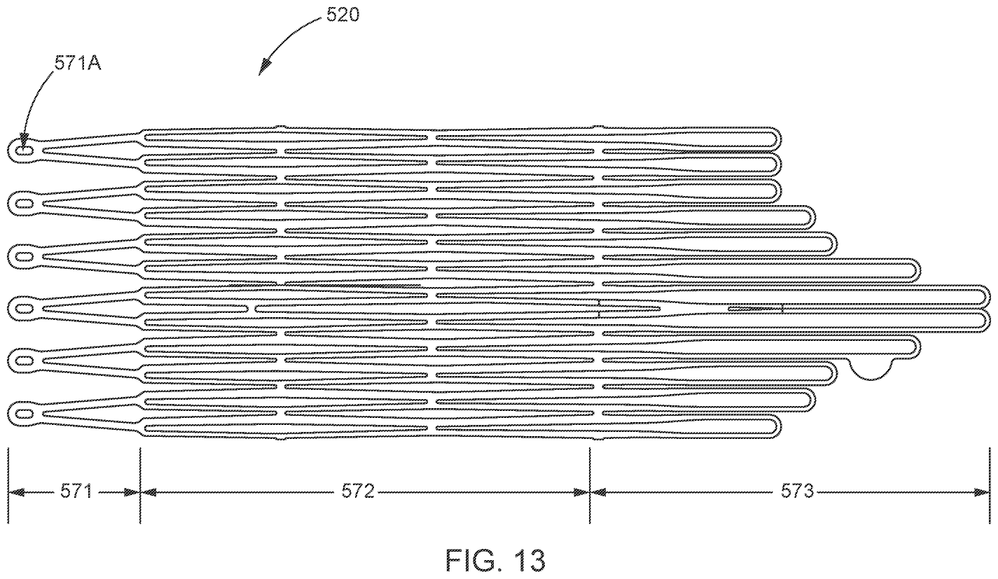

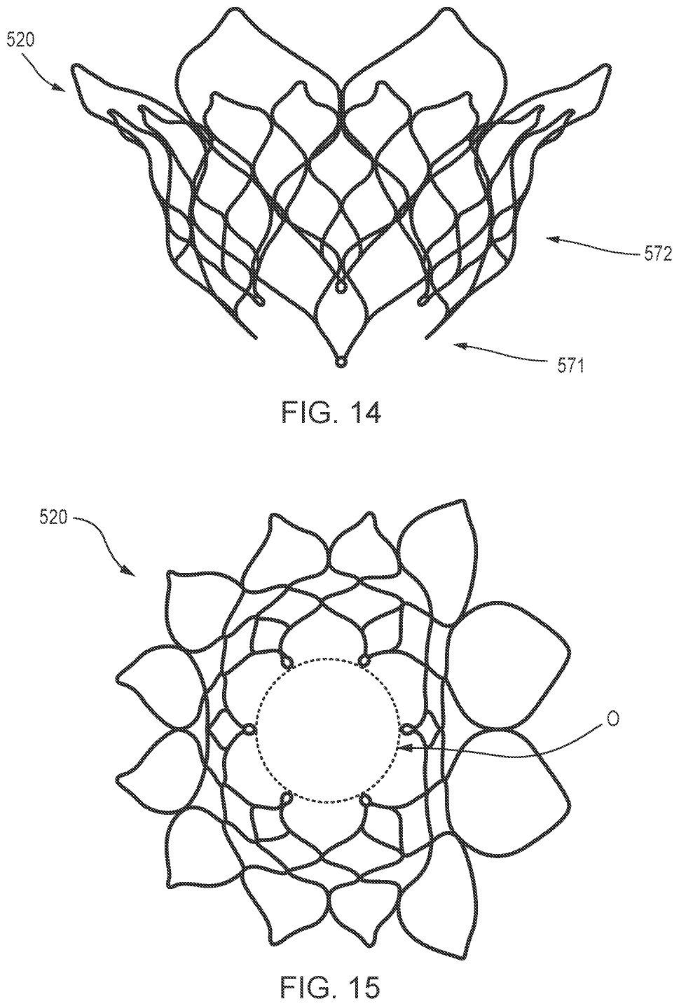

Outer frame 520 is shown in a fully deformed, i.e. the final, deployed configuration, in side view and top view in FIGS. 14 and 15, respectively. As best seen in FIG. 15, the lower end of coupling portion 571 forms a roughly circular opening (identified by “O” in FIG. 15). The diameter of this opening preferably corresponds approximately to the diameter of body portion 542 of inner frame 550, to facilitate coupling of the two components of valve 500.

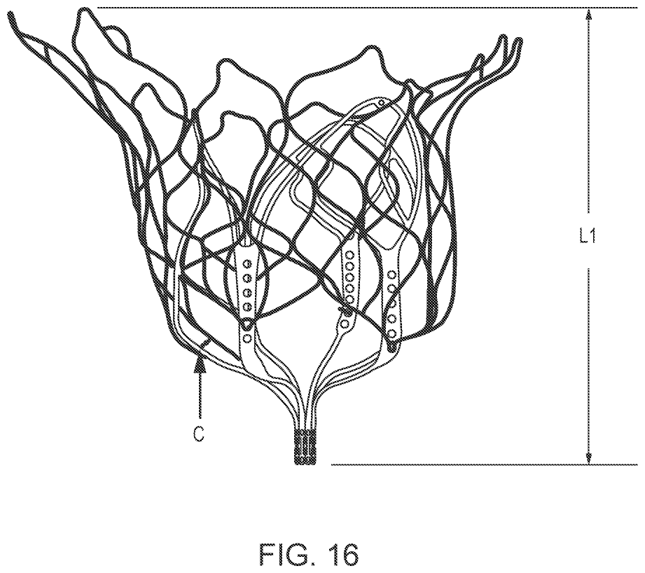

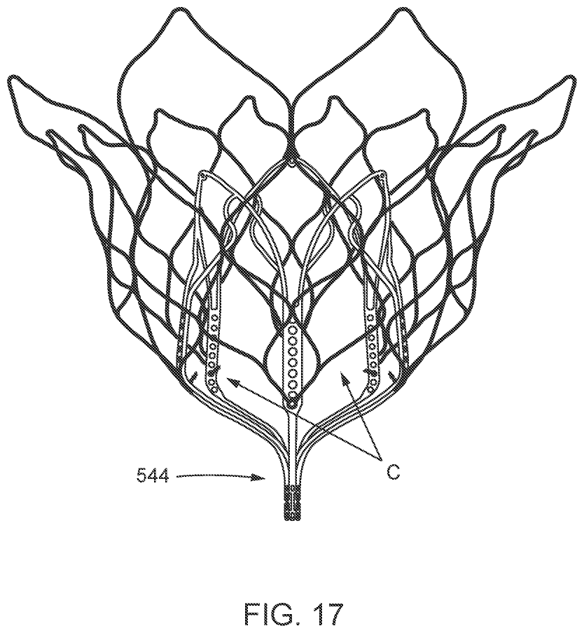

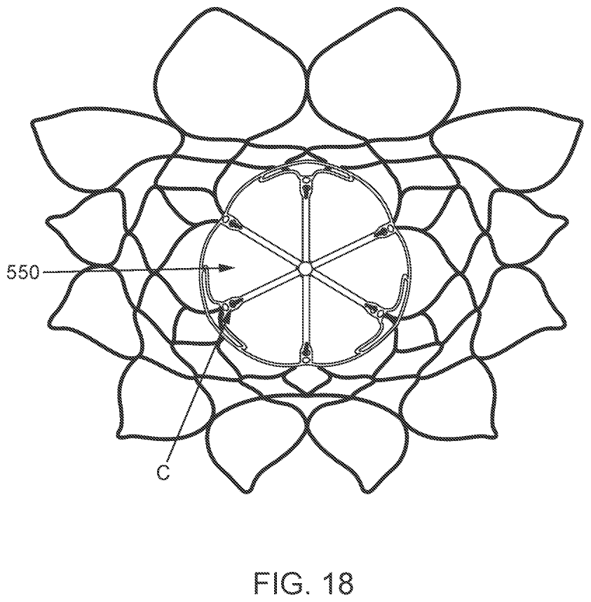

Outer frame 520 and inner frame 550 are shown coupled together in FIGS. 16-18, in front, side, and top views, respectively. The two frames collectively form a structural support for a prosthetic valve such as valve 500. The frames support the valve leaflet structure (e.g., leaflets 570) in the desired relationship to the native valve annulus, support the coverings (e.g., outer covering 530, inner covering 532, outer covering 560) for the two frames to provide a barrier to blood leakage between the atrium and ventricle, and couple to the tether (e.g., tether assembly 590) (by the inner frame 550) to aid in holding the prosthetic valve in place in the native valve annulus by the tether connection to the ventricle wall. The outer frame 520 and the inner frame 550 are connected at six coupling points (representative points are identified as “C”). In this embodiment, the coupling points are implemented with a mechanical fastener, such as a short length of wire, passed through an aperture (such as aperture 571A) in coupling portion 571 of outer frame 520 and corresponding openings in longitudinal posts (such as post 542A) in body portion 542 of inner frame 550. Inner frame 550 is thus disposed within the outer frame 520 and securely coupled to it.

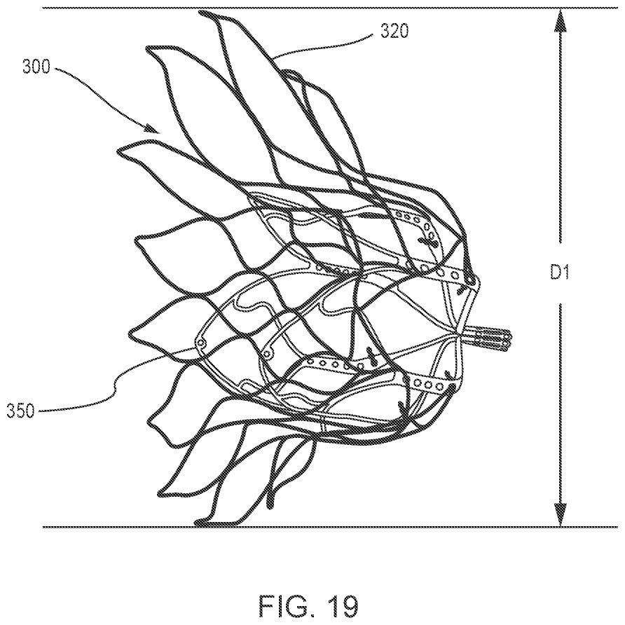

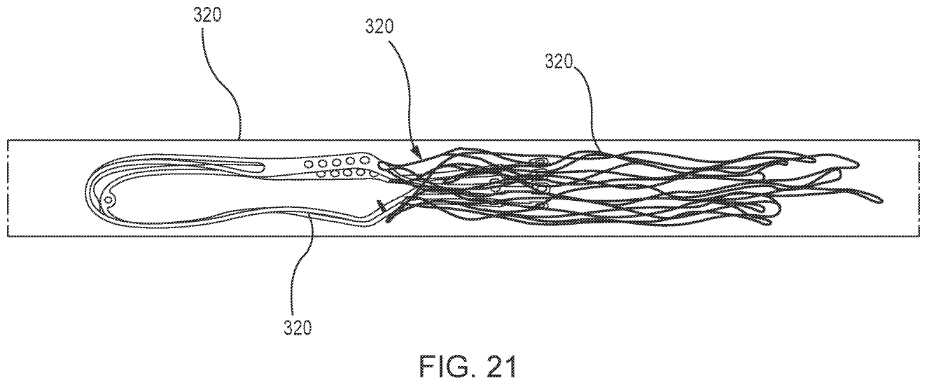

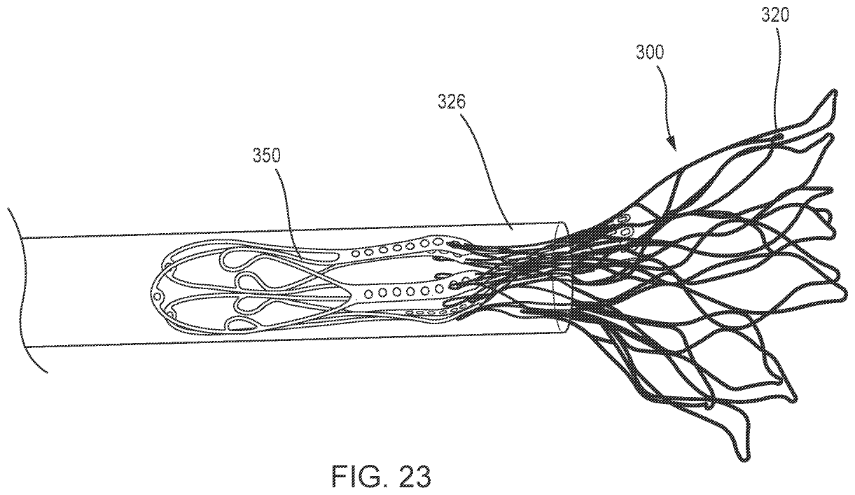

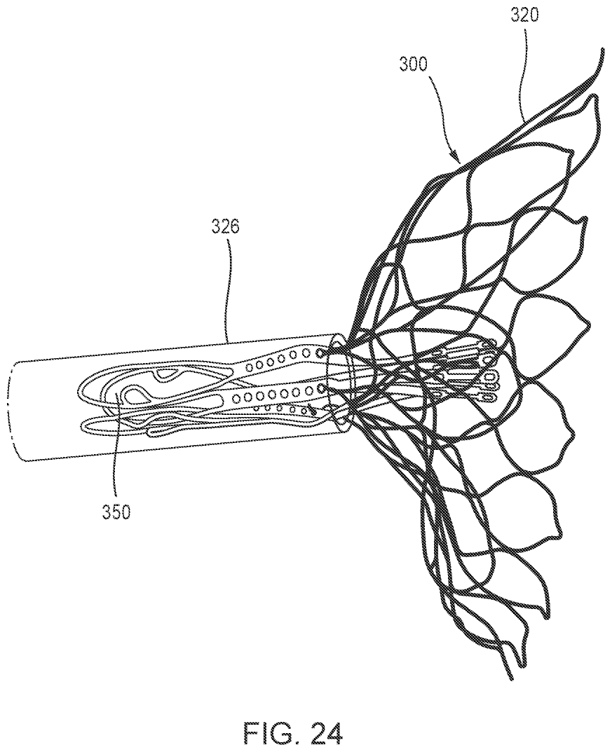

FIGS. 19-25 illustrate a method of reconfiguring a prosthetic heart valve 300 (e.g., prosthetic mitral valve) prior to inserting the prosthetic heart valve 300 into a delivery sheath 326 (see, e.g., FIGS. 21-25) for delivery into the heart via the femoral vein. The prosthetic heart valve 300 (also referred to herein as “valve”) can be constructed the same as or similar to, and function the same as or similar to the valve 500 described above. Thus, some details regarding the valve 300 are not described below. It should be understood that for features and functions not specifically discussed, those features and functions can be the same as or similar to the valve 500.

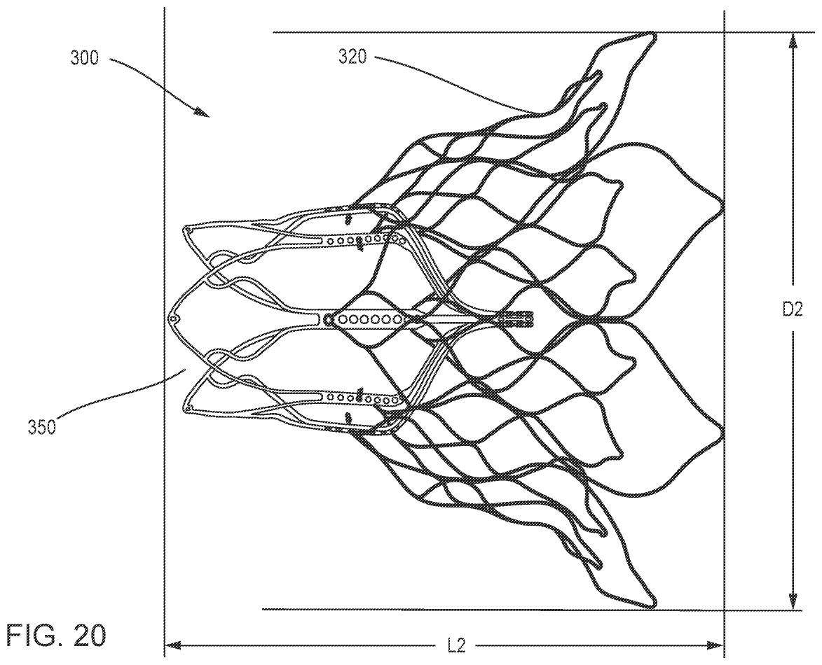

As shown in FIG. 19, the valve 300 has an outer frame 320 and an inner frame 350. As discussed above for valves 200 and 500, the outer frame 320 and the inner frame 350 of valve 300 can each be formed with a shape-memory material and have a biased expanded or deployed configuration. The outer frame 320 and the inner frame 350 can be moved to a collapsed or undeployed configuration for delivery of the valve 300 to the heart. In this example method of preparing the valve 300 for delivery to the heart, the outer frame 320 of the valve 300 is first disposed in a prolapsed or inverted configuration as shown in FIG. 20. Specifically, the elastic or superelastic structure of outer frame 320 of valve 300 allows the outer frame 320 to be disposed in the prolapsed or inverted configuration prior to the valve 300 being inserted into the lumen of the delivery sheath 326. As shown in FIG. 20, to dispose the outer frame 320 in the inverted configuration, the outer frame 320 is folded or inverted distally such that the outer frame 320 is pointed away from the inner frame 350. In this inverted configuration, the overall outer perimeter or outer diameter of the valve 300 is reduced and the overall length is increased. For example, the diameter D1 shown in FIG. 19 is greater than the diameter D2 shown in FIG. 20, and the length L1 in FIG. 16 is less than the length L2 in FIG. 20. With the outer frame 320 in the inverted configuration, the valve 300 can be placed within a lumen of a delivery sheath 326 as shown in FIG. 21 for delivery of the valve 300 to the left atrium of the heart. By disposing the outer frame 320 in the inverted configuration, the valve 300 can be collapsed into a smaller overall diameter, i.e. placed in a smaller diameter delivery sheath, than would be possible if the valve 300 in the configuration shown in FIG. 19 were collapsed radially. This is because in the configuration shown in FIG. 19, the two frames are concentric, and thus the outer frame 320 must be collapsed around the inner frame 350, whereas in the configuration shown in FIG. 20, the two frames are coaxial but not concentric, such that the outer frame 320 can be collapsed without needing to accommodate the inner frame 350 inside it.

The procedure to deliver the valve 300 to the heart can be the same as or similar to the procedure described with reference to FIGS. 1-6. In this embodiment, the valve 300 is not partially deployed outside of the lumen of the delivery sheath 326 prior to being inserted into a femoral puncture, through the femoral vein, through the inferior vena cava, into the right atrium, through the septum Sp and into the left atrium LA of the heart. With the distal end portion of the delivery sheath 326 disposed within the left atrium of the heart, the valve 300 can be deployed outside of the delivery sheath 326. For example, although not shown, a tether such as tether 236 described above for valve 200 can be attached to the valve 300 and used to pull the valve 300 out of the lumen of the delivery sheath 326. Alternatively, or in addition to, a pusher device (not shown) can be used to deploy the valve 300. Thus, as described above for valve 200, the valve 300 can be deployed by pushing with the pusher device, pulling with the tether, or both.

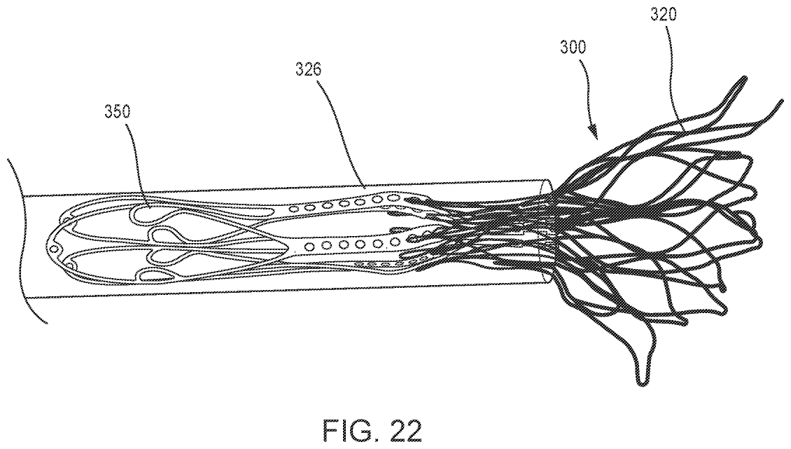

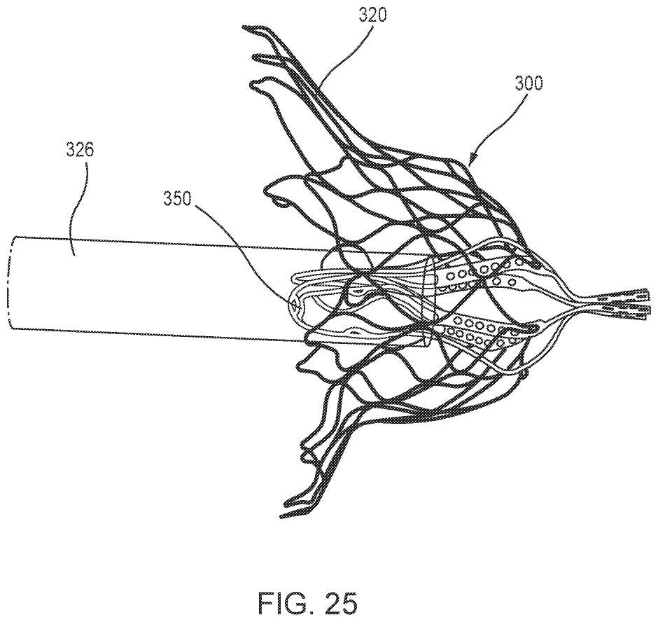

As the valve 300 exits the lumen of the delivery sheath 326, the outer frame assembly 310 exits first in its inverted configuration as shown in the progression of FIGS. 22-24. After the outer frame assembly 310 is fully outside of the lumen of the delivery sheath 326, the outer frame 320 can revert to its expanded or deployed configuration as shown in FIG. 25. In some embodiments, the pusher device and/or the tether can be used to aid in the reversion of the outer frame assembly 310. The valve 300 can continue to be deployed until the inner frame 350 is fully deployed with the left atrium and the valve 300 is in the expanded or deployed configuration (as shown in FIG. 19).

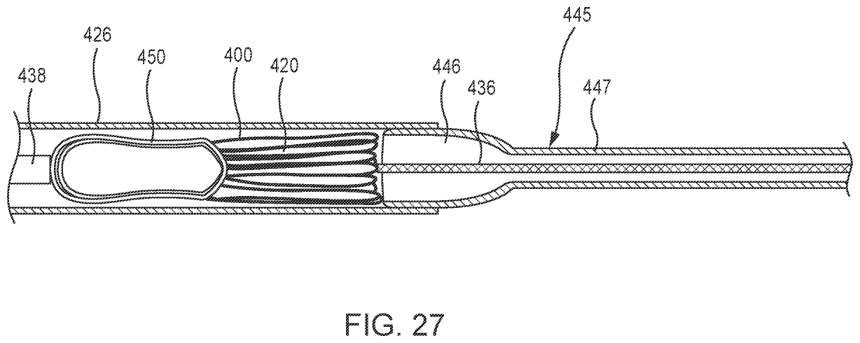

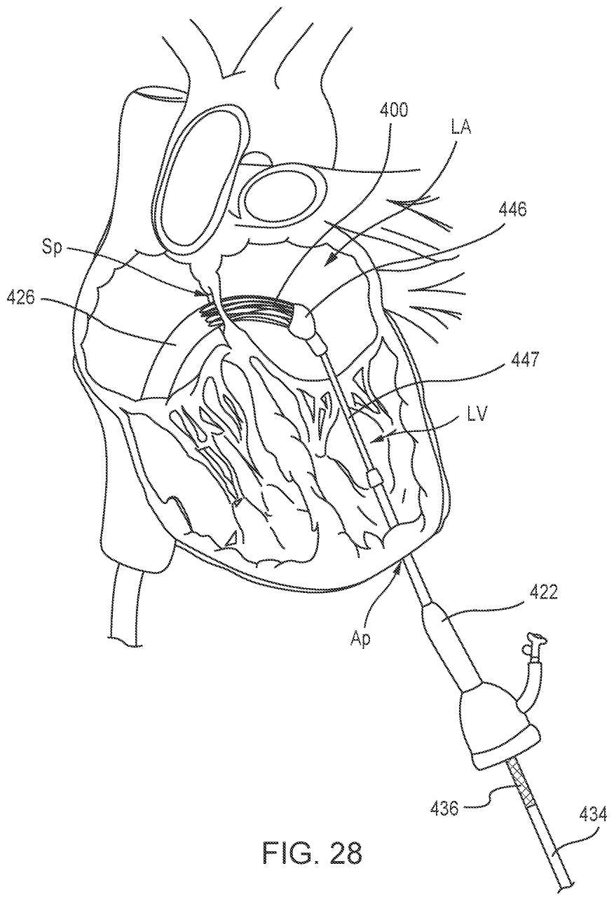

FIGS. 27 and 28 illustrate an optional balloon dilator device that can be used during a procedure for transfemoral delivery of a prosthetic heart valve to the heart. FIG. 27 illustrates a valve 400 disposed within a lumen of a delivery sheath 426. The valve 400 can be constructed the same as or similar to, and function the same as or similar to, the valves 200, 500 and 300 described above. For example, the valve 400 can include an outer frame 420 and an inner frame 450 as described above for previous embodiments. A tether 436 can be coupled to the valve 400 and a valve leader member 434 (see FIG. 28) can be coupled to the tether 436.

In this embodiment, to deliver the valve 400, a leader tube (not shown) can be inserted through an apical puncture and extended through the heart and out through a femoral vein access site. A valve leader member 434 coupled to a tether 436 can be inserted through the femoral end of the leader tube and extended out the apical end of the leader tube, as described above with respect to FIGS. 1-6. The valve 400 can be loaded into the distal end of a lumen of a delivery sheath 426 either before or after the tether 436 and valve leader member 434 are looped through the patient. A balloon dilator device 445 can then be advanced along the valve leader member 434 from the apical end, through the heart, through the femoral vein and out the femoral access site.

The balloon dilator device 445 includes a balloon member 446 that can be disposed at least partially within the distal end portion of the lumen of the delivery device 426, and distal of the valve 400, as shown in FIG. 27. The balloon dilator device 445 also includes an elongate member 447 coupled to the balloon member 446 and that defines an inflation lumen in fluid communication with an interior of the balloon member 446. The elongate member 447 can be coupled to a source of an inflation medium (not shown) configured to supply the inflation medium to the balloon member 446. With the balloon dilator device 445 coupled to the delivery sheath 426 as shown in FIG. 27, the balloon member 446 can be inflated. The delivery sheath 426 can then be inserted through the femoral access site and advanced through the femoral vein, through the inferior vena cava, into the right atrium, through the septum Sp and into the left atrium LA as shown in FIG. 28. The balloon member 446 provides a smooth surface to aid in maneuvering the delivery sheath 426 through the femoral vein and the septum and into the heart. With the distal end portion of the delivery sheath 426 disposed within the left atrium LA, the balloon member 446 can be deflated and removed through the apical access site. The valve 400 can then be deployed and positioned within the mitral annulus as described above for FIGS. 1-6. For example, a pusher device 438 (see FIG. 27) can be used to push the valve 400 out of the lumen of the delivery sheath 426 and/or the tether 436 coupled to the valve 400 can be pulled.

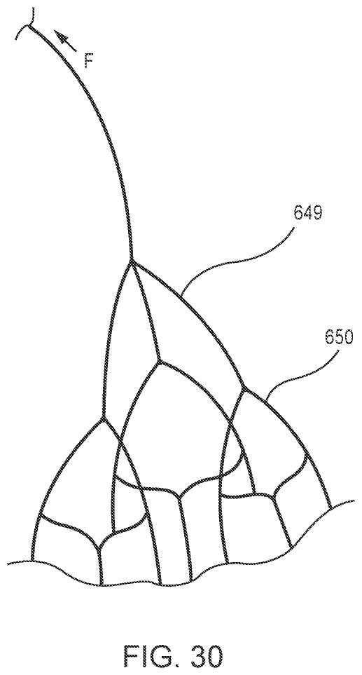

FIGS. 29 and 30 illustrate an optional wire assist structure that can be used during a procedure to deliver a prosthetic heart valve transfemorally as described above for previous embodiments. A wire assist structure 649 can be releasably coupled to a valve 600 as shown in FIG. 29. The valve 600 can be constructed the same as or similar to, and function the same as or similar to, the valves described above for previous embodiments. For example, the valve 600 can include an outer frame 620 and an inner frame 650. The wire assist structure 649 can be releasably coupled to the inner frame 650 as best shown in FIG. 30. For example, releasable connectors (not shown) can be used to couple the wire assist structure 649 to the inner frame 650.

In use, the wire assist structure 649 can be movably disposed within a delivery sheath 626 used to deliver the valve 600 to the heart. The wire assist structure 649 can hold the inner frame 650 and allow for positioning control of the valve 600 (i.e., clocking and advancement) while the outer frame 650 of the valve 600 is fully expanded, which allows the valve 600 to be functioning during the positioning phase. When the valve 600 is in the desired final position, the wire assist structure 649 can be released from the inner frame 650 and removed with the delivery sheath 626.

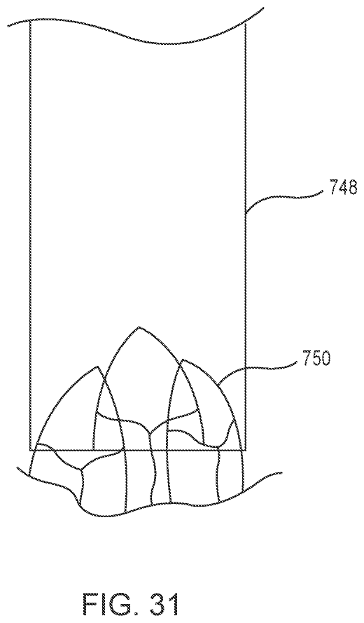

FIG. 31 illustrates another optional assist member that can be used during a procedure to deliver a prosthetic heart valve transfemorally. An assist member 748 can be in the form of a tubular member defining a lumen with a diameter sized to receive at least a portion of the inner frame 750 of a valve 700. The valve 700 can be constructed the same as or similar to, and function the same as or similar to, the valves described above for previous embodiments. For example, the valve 700 can include an outer frame (not shown) and the inner frame 750 as described above for previous embodiments.

In use, the assist member 748 can be movably disposed within a delivery sheath (not shown) used to deliver the valve 700 and be disposed over at least a portion of the inner valve assembly 740. As with the wire assist structure 649, the assist member 748 can hold the inner frame 750 in a small compact configuration and allow for positioning control of the valve 700 (i.e., clocking and advancement) while the outer frame of the valve 700 is being expanded. This can in some cases allow the valve 700 to be functioning (or at least partially functioning) during the positioning phase of the valve 700. With the inner frame 750 held in a compact or small diameter form factor, the valve 700 can be more easily positioned to help seal the annulus with the outer frame (not shown) of the valve 700. When the valve 700 is in the desired final position, the assist member 748 can be removed.

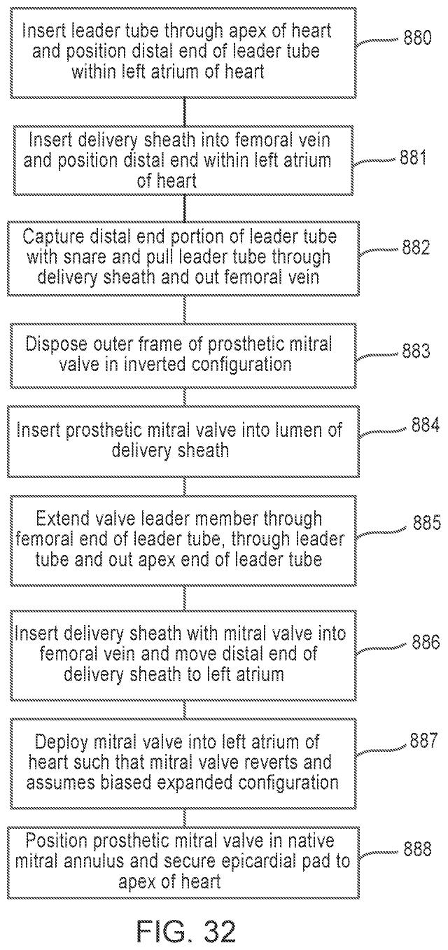

FIG. 32 is a flowchart illustrating a method of deploying a prosthetic mitral valve to a heart using a transfemoral delivery approach. The method includes at 880, inserting a leader tube through an access site on the skin of the patient, through an access puncture site on the apex of the heart, and positioning a distal end portion of the leader tube in the left atrium of the heart. At 881, inserting a delivery sheath with a snare device coupled thereto through an access site into the femoral vein and into the left atrium of the heart. At 882, the leader tube is captured with the snare device, and pulled through the femoral vein such that the leader tube extends between the apex of the heart and the entry to the femoral vein. At 883, an outer frame of a prosthetic mitral valve is disposed in an inverted configuration when the mitral valve is in a biased expanded configuration. For example, the prosthetic mitral valve can be formed with a shape-memory material and have a biased expanded configuration.

At 884, after inverting the outer frame, the prosthetic mitral valve is inserted into a lumen of a delivery sheath such that the prosthetic mitral valve is moved to a collapsed configuration. The delivery sheath can be the same delivery sheath as used with the snare device or a different delivery sheath. At 885, a valve leader member is inserted to the leader tube at the femoral end of the leader tube, and moved through the leader tube until the valve leader member exits the leader tube outside of the apex of the heart. A proximal end of the valve leader member is coupled to a tether line that in turn is coupled to the prosthetic mitral valve and disposed within the delivery sheath. At 886, the delivery sheath is inserted into the femoral vein and moved through the femoral vein and through a septum of a heart until a distal end portion of the delivery sheath is disposed in the left atrium of the heart. At 887, the prosthetic mitral valve is moved distally out of the delivery sheath such that the inverted outer frame of the prosthetic mitral valve reverts, and the prosthetic mitral valve assumes its biased expanded configuration. At 888, the prosthetic mitral valve is positioned within a mitral annulus of the heart and optionally an epicardial pad device can be secured to the apex of the heart to maintain the prosthetic mitral valve in the desired position (e.g., orientation) within the mitral annulus. In some embodiments, rather than securing the prosthetic mitral valve with a tether and epicardial pad, the prosthetic mitral valve can be secured with clips or other coupling methods to a portion(s) of the ventricular wall of the heart.

FIGS. 33-37 illustrate an embodiment of an expandable epicardial pad device that can be used to secure a tether attached to a prosthetic mitral valve to the heart, for example, at the apex of the heart. An epicardial pad device 939 (also referred to herein as “epicardial pad” or “pad”) can be used, for example, during a procedure to deliver a prosthetic heart valve transfemorally as described herein. The epicardial pad 939 can be formed with a small profile such that the epicardial pad 939 can be delivered to the exterior of the heart via a small incision and a small diameter delivery catheter or sheath 963 (see FIGS. 33 and 34). In some embodiments, the delivery sheath 963 can have a diameter, for example, in the range of 3-5 mm An inner delivery sheath 964 can be movably disposed within a lumen of the delivery sheath 963 and used to hold the tether 936 while the epicardial pad 939 is being deployed as described in more detail below.

As shown in FIGS. 33 and 34 the epicardial pad 939 includes a frame member 961 and a fabric cover 962. The frame member 961 can be formed with, for example a shape-memory material such as Nitinol® such that the epicardial pad 939 can have a biased expanded configuration as shown in FIGS. 34 and 36, and can be moved to a collapsed configuration as shown in FIG. 33. For example, as shown in FIG. 33 the epicardial pad 939 can be placed within a lumen of the delivery sheath 963 to move the epicardial pad 939 to the collapsed configuration. The fabric cover 962 can be formed with various suitable material(s) such as, for example, polyester, polyethylene or ePTFE.

In use, after a prosthetic mitral valve has been deployed within the heart H via a transfemoral delivery approach as described herein, the tether 936 attached the prosthetic valve (not shown) can extend outside the apex of the heart. The epicardial pad 939 can be used to secure the tether 936 and prosthetic valve in a desired position. With the tether 936 extending outside of the heart, the tether 936 can be threaded through a center opening of the epicardial pad 939 and through a lumen of the inner delivery sheath 964, as shown in FIGS. 33 and 34. The outer delivery sheath 963 can be laced over the inner delivery sheath 964 and the epicardial pad 939 to collapse the epicardial pad 939 as shown in FIG. 33. As described above, the outer delivery sheath 964 can have a relatively small outer diameter such that it can be inserted through a small incision in the skin of the patient. When the distal end of the delivery sheath 963 is at a desired location near the apex of the heart, the epicardial pad 939 can be moved outside of the delivery sheath 963 such that the epicardial pad 939 can assume its biased expanded configuration as shown in FIGS. 34 and 36. For example, to move the epicardial pad 939 outside of the lumen of the delivery sheath 963, the delivery sheath 963 can be moved proximally, such that the deliver sheath 963 is removed from epicardial pad 939. Alternatively, the epicardial pad 939 can be moved distally outside of the lumen of the delivery sheath 963. For example, a push rod (not shown) can be used, or the inner delivery sheath 964 in which the tether 936 is disposed can be used to move or push the epicardial pad 939 out of the delivery sheath 963.

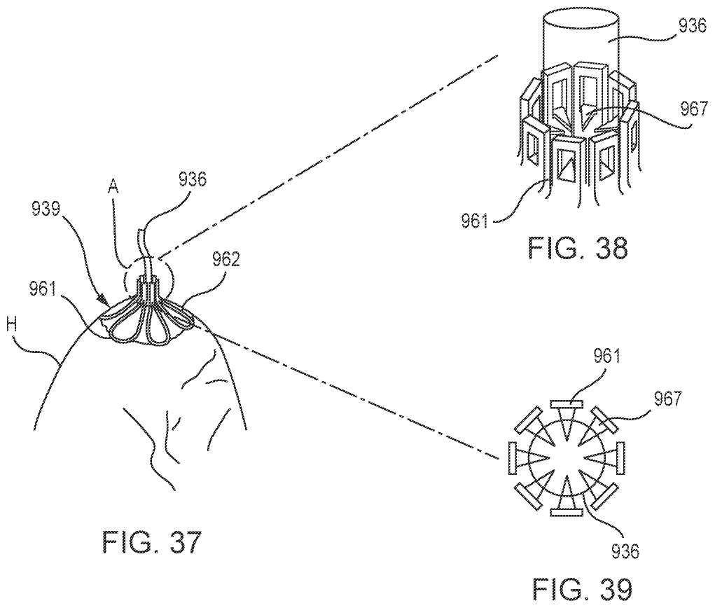

Prior to moving the expanded epicardial pad 939 into position on the apex of the heart, conventional purse string sutures 965 at the incision through which the tether 936 extends out of the heart at the apex of the heart can be closed. The epicardial pad 939, in the expanded configuration, can then be positioned on the apex of the heart. In this embodiment, the epicardial pad 939 includes an integral locking mechanism 966 as shown in FIGS. 37-39. The locking mechanism can be formed integrally with the frame member 961 and can include barbs 967. As shown in FIGS. 33 and 34, the tether 936 can be inserted through a lumen of the inner delivery sheath 964 such that the delivery sheath 964 can prevent the barbs 967 from contacting the tether 936. For example, the tether 936 can be threaded into the inner delivery sheath 964 prior to the inner delivery sheath 964 and tether 936 being inserted through the center opening of the epicardial pad 939. Thus, the inner delivery sheath 964 can protect the tether 936 from the barbs 967 of the locking mechanism 966 during deployment of the epicardial pad 939. When the epicardial pad 939 is deployed at the desired position on the heart, the inner delivery sheath 964 can be removed uncovering the tether 936 and allowing the barbs 967 to engage or pierce the tether 936 as shown in FIGS. 38 and 39. The barbs 968 can hold or lock the tether 936 and epicardial pad 939 in the desired position. The barbs 968 can be oriented at various different angles relative to a longitudinal axis of the epicardial pad 939, such as, for example, between 45-120 degrees.

In alternative embodiments, other methods of securing the epicardial pad 939 to the heart can be used. For example, in an embodiment in which the epicardial pad 939 does not include an integrated locking mechanism as described above, the distal end portion of the tether 936 can be tied or another securing device such as a clip or locking pin can be used.

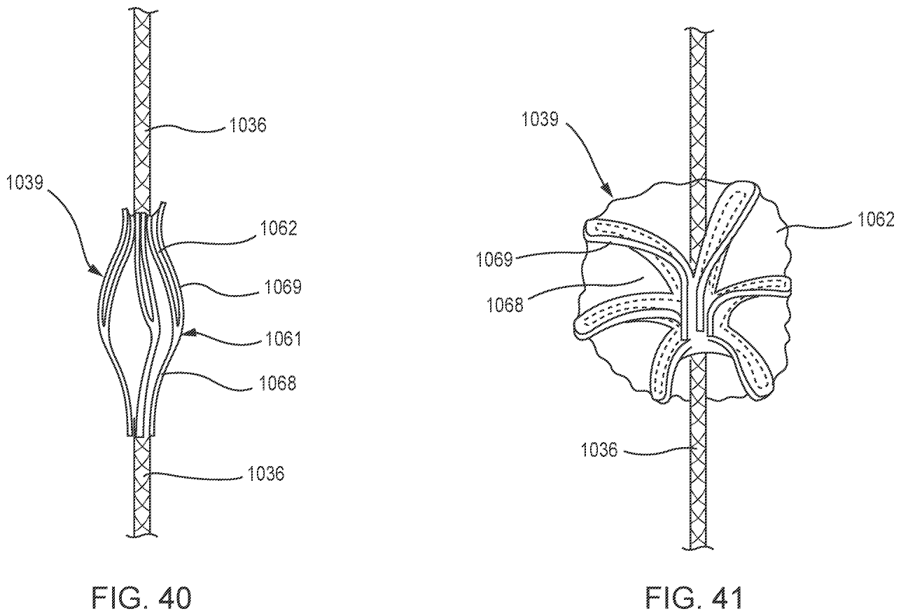

FIGS. 40-42 illustrate another embodiment of an expandable epicardial pad device that can be used to secure a tether attached to a prosthetic mitral valve to the heart, for example, at the apex of the heart. An epicardial pad device 1039 (also referred to herein as “epicardial pad” or “pad”) can be used, for example, during a procedure to deliver a prosthetic heart valve transfemorally as described herein. The epicardial pad 1039 can be formed with a small profile such that the epicardial pad 1039 can be delivered to the exterior of the heart via a small incision and a small diameter delivery catheter or sheath (not shown) as described above for epicardial pad 939.

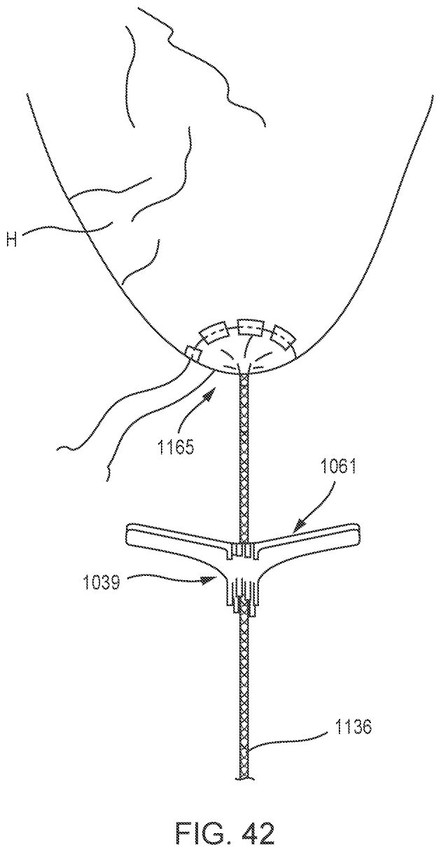

As shown in FIGS. 40-42, the epicardial pad 1039 includes a frame member 1061 and a fabric cover 1062. In this embodiment, the frame member 1061 includes a first frame portion 1068 and a second frame portion 1069. As with the previous embodiment, the frame member 1061 can be formed with, for example a shape-memory material such as Nitinol®, such that the epicardial pad 1039 can have a biased expanded configuration as shown in FIGS. 41 and 42, and can be moved to a collapsed configuration as shown in FIG. 40. For example, although not shown for this embodiment, the epicardial pad 1039 can be placed within a lumen of a delivery sheath to collapse or move the epicardial pad 1039 to the collapsed configuration. In the expanded configuration, the second frame portion 1069 expands within an interior region defined by the first frame portion 1068 as best shown in FIG. 41. In other words, the second frame portion 1069 and the first frame portion 1068 form a double-layer flower-like shape. The fabric cover 1062 can be formed with, for example, various suitable material(s) such as, for example, polyester, polyethylene or ePTFE, as described above for fabric cover 962.

In use, after a prosthetic mitral valve has been deployed within the heart H (FIG. 42), for example, via a transfemoral delivery approach as described herein, the tether 1036 attached the prosthetic valve (not shown) can extend outside the apex of the heart. The epicardial pad 1039 can be used to secure the tether 1036 and prosthetic valve in a desired position. With the tether 1036 extending outside of the heart, the tether 1036 can be threaded through a lumen of an inner delivery sheath, such as inner delivery sheath 964 described above, and through a center opening of the epicardial pad 1039. An outer delivery sheath (not shown) can be placed over the inner delivery sheath such that the epicardial pad 1039 to collapse the epicardial pad 1039. As described above, the outer delivery sheath can have a relatively small outer diameter such that it can be inserted through a small incision in the skin of the patient. When the distal end of the delivery sheath is at a desired location near the apex of the heart, the epicardial pad 1039 can be moved outside of the delivery sheath 963 such that the epicardial pad 1039 can assume its biased expanded configuration as shown in FIGS. 41 and 42 as described above for epicardial pad 939.

Prior to moving the expanded epicardial pad 1039 into position on the apex of the heart, conventional purse string sutures 1065 at the incision through which the tether 1036 extends out of the heart at the apex of the heart can be closed. The epicardial pad 1039, in the expanded configuration, can then be positioned on the apex of the heart. The epicardial pad 1039 can include an integral locking mechanism, similar to or the same as locking mechanism 966 described above to secure or lock the tether 1036 and epicardial pad 1039 in position on the heart. In alternative embodiments, other methods of securing the epicardial pad 1039 to the heart can be used. For example, as described above, the distal end portion of the tether 1036 can be tied or another securing device such as a clip or locking pin can be used.

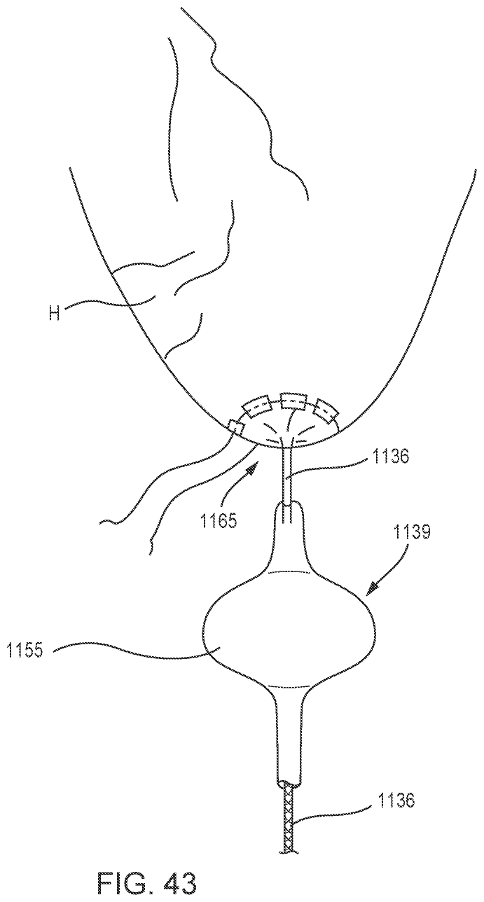

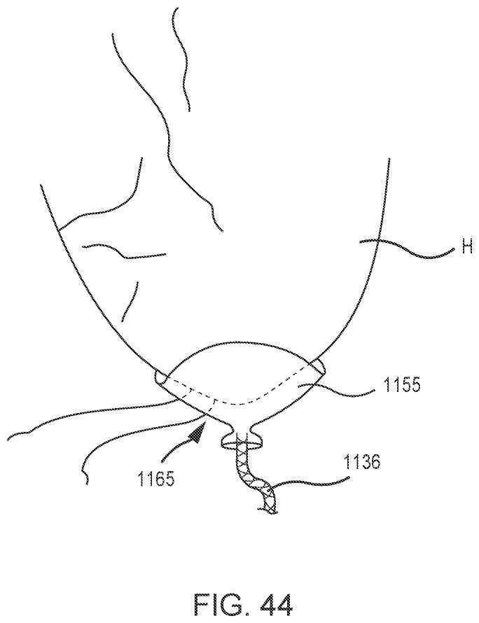

FIGS. 43 and 44 illustrate an expandable epicardial pad device 1139 according to another embodiment. The epicardial pad device 1139 can be used in the same or similar manner as described for previous embodiments to secure a tether attached to a prosthetic mitral valve to the heart, for example, at the apex of the heart. The epicardial pad device 1139 (also referred to herein as “epicardial pad” or “pad”) can be used, for example, during a procedure to deliver a prosthetic heart valve transfemorally as described herein. In this embodiment, the epicardial pad device 1139 includes a balloon member 1155. The balloon member 1155 can be small in size such that the balloon member 1155 can be delivered to the exterior of the heart via a small incision and a small diameter delivery catheter or sheath (not shown) as described above for previous embodiments.

The balloon member 1155 can define an inner lumen through which the tether 1136 can be inserted. The epicardial pad 1139 can also include an inflation lumen through which an inflation medium can be communicated to and from the balloon member 1155. For example, the inflation lumen (not shown) can be defined by the balloon member 1155 or by a separate inflation line (not shown) in fluid communication with an interior of the balloon member 1155.

In use, after a prosthetic mitral valve has been deployed within the heart H (FIG. 42), for example, via a transfemoral delivery approach as described herein, the tether 1136 attached the prosthetic valve (not shown) can extend outside the apex of the heart. With the tether 1136 extending outside of the heart, the tether 1136 can be threaded or inserted through the lumen of the balloon member 1155 as described above. The balloon member 1155 can be inflated or deflated when the tether 1136 is inserted into the balloon lumen. The balloon member 1155 can be collapsed or deflated (not shown) and then placed within a lumen of a delivery sheath (not shown). The delivery sheath can be inserted through a small incision in the skin of the patient and a distal end of the delivery sheath disposed at a desired location near the apex of the heart, The epicardial pad 1139 (i.e., balloon member 1155) can be moved outside of the delivery sheath and then can be inflated as shown in FIG. 43.

Purse string sutures 1165 at the incision through which the tether 1136 extends out of the heart at the apex of the heart can be closed prior to positioning the epicardial pad 1139 on the apex. Prior to positioning the balloon member 1155 on the apex of the heart, the balloon member 1155 can be partially deflated or fully deflated. The balloon member 1155 is then moved distally into contact with the heart where it can collapse inwardly upon itself to form a cup shape as the balloon member 1155 is pushed against the heart, as shown in FIG. 44. The epicardial pad 1139 and tether 1136 can be secured in the desired position with, for example, clip(s) or a locking pin(s) or by tying the tether 1136. In some embodiments, the balloon member 1155 is secured by adhesively coupling the balloon member 1155 to the tether 1136 such that the balloon member 1155 is prevented from moving relative to the tether 1136. In some embodiments, the balloon member 1155 can be adhesively coupled to the tether 1136 and also adhesively coupled to the heart. In some embodiments, the balloon member 1155 is fully deflated and can be filled with an adhesive or a cement material to add strength and rigidity to the balloon member 1155.

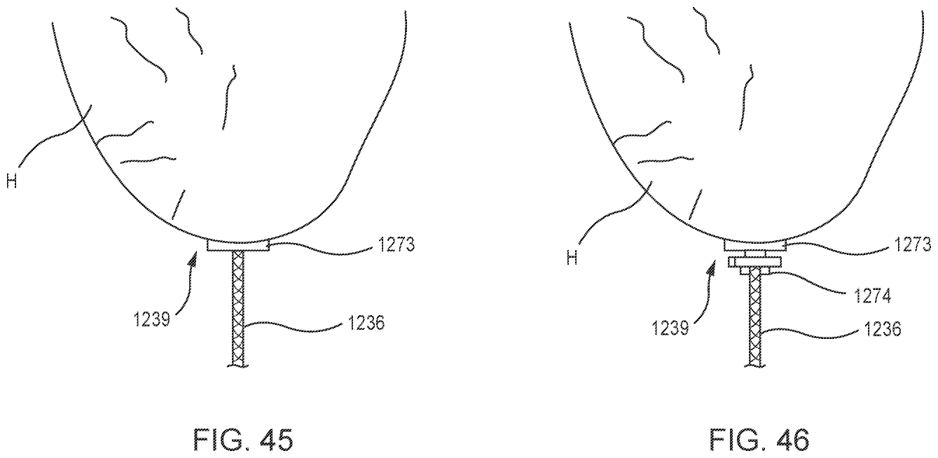

FIGS. 45-47 illustrate yet another embodiment of an epicardial pad device that can be used to secure a tether attached to a prosthetic mitral valve to the heart, for example, at the apex of the heart. The epicardial pad device 1239 (also referred to herein as “epicardial pad” or “pad”) can be used, for example, during a procedure to deliver a prosthetic heart valve transfemorally as described herein. In this embodiment, the epicardial pad device 1239 includes multiple stackable pad members 1273 that can be sized such that each stackable pad member 1273 can be delivered separately to the exterior of the heart via a small incision and a small diameter delivery catheter or sheath (not shown). When all of the stackable pad members 1273 are implanted and attached to the heart, the stackable pad members 1273 can define a total surface area of, for example, 2 cm. The stackable pad members 1273 can be formed with, for example, suitable polymer or metal materials such as, for example, PEEK plastic, or stainless steel such as, for example, MP35N stainless steel.

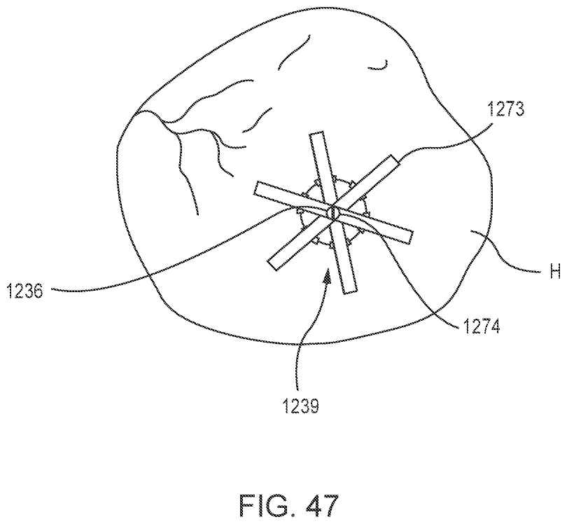

In use, after a prosthetic mitral valve has been deployed within the heart H, for example, via a transfemoral delivery approach as described herein, the tether 1236 attached the prosthetic valve (not shown) can extend outside the apex of the heart. With the tether 1236 extending outside of the heart, a first stackable pad member 1273 can be slid onto the tether 1236. For example, the stacking members 1273 can define a through-hole in which the tether 1236 can be received. The first stackable pad member 1273 can be slid or moved distally along the tether 1236 until it contacts the surface of the heart H as shown in FIG. 45. A second stackable pad member 1273 can then be slid distally along the tether 1236 until it contacts the first stackable pad member 1273 and then a third stackable pad member 1273 can be slid distally along the tether 1236 until it contacts the second stackable pad member 1273 as shown in FIG. 45. Each stackable pad member 1273 can be oriented at a different angle relative to the tether 1236 as shown in FIG. 47. Using three separate stackable pad members 1273 in this manner can distribute the forces against the surface of the heart more evenly than a single stackable pad member 1273. After the three stackable pad members 1273 have been positioned against the heart, a locking pin 1274 can be inserted laterally through the tether 1236 to secure the stackable pad members 1273 against the surface of the heart. In some embodiments, it may be desirable to insert a locking pin after each stackable pad member 1273 has been positioned.

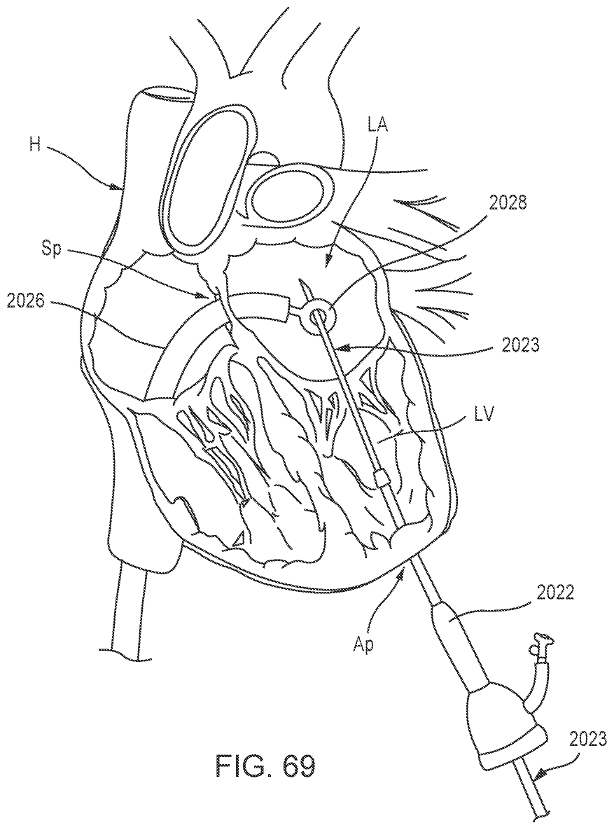

FIGS. 48-51B illustrate an optional stopper tube device, also referred to as a valve positioning device or a valve alignment device or included as part of a valve positioning device or valve alignment device that can be used during a procedure to deliver a prosthetic heart valve to a heart of a patient. For example, a stopper tube device as described herein can be used during a procedure to deliver a prosthetic heart valve to a heart using a variety of different delivery approaches, such as, a transfemoral approach as described above for previous embodiments, and as described below with respect to FIGS. 69-74. Other delivery approaches can be used as described above, such as, a transatrial approach or a transjugular approach. The stopper tube device can be used to assist in the positioning of the prosthetic valve within the heart and prevent the prosthetic valve from entering the left ventricle prior to delivering the prosthetic valve to the annulus of the heart. As shown in FIGS. 48-51, a prosthetic valve 1300 can be constructed the same as or similar to, and function the same as or similar to, the prosthetic valves described above for previous embodiments. For example, the prosthetic valve 1300 can include an outer frame and an inner frame. The prosthetic valve 1300 can be delivered to the heart H as described above for previous embodiments. For example, the prosthetic valve 1300 can be placed in the distal end of a delivery sheath 1326 and the delivery sheath 1326 can be introduced through a femoral vein puncture and extended through the inferior vena cava IVC, into the right atrium, and then through a transseptal puncture of the septum Sp of the heart H, and into the left atrium LA of the heart H.

With the distal end portion of the delivery sheath 1326 disposed within the left atrium LA of the heart, the prosthetic valve 1300 can be deployed outside of the delivery sheath 1326. A tether 1336 can be coupled to the prosthetic valve 1300 and used to pull the prosthetic valve 1300 out of the lumen of the delivery sheath 1326 as described previously. In some embodiments, the tether 1336 can be formed, for example, as a braided rope or cord. Alternatively, or in addition to, a pusher device (not shown) (e.g., pusher device 238), as described above can be used to deploy the prosthetic valve 1300. The tether 1336 can be extended through the annulus, through the left ventricle LV and exit the heart at the apex Ap.

In this embodiment, the tether 1336 is threaded or inserted through a stopper tube device 1376 (also referred to as “stopper tube”) as shown in FIGS. 48-51. The stopper tube 1376 is then inserted through the apex Ap of the heart and a distal end of the stopper tube 1376 is extended through the left ventricle LV, through the annulus and into the left atrium LA. The stopper tube 1376 at this point in the delivery procedure can be used, for example, to prevent the prosthetic valve 1300 from entering into the left ventricle LV too early or too deep.

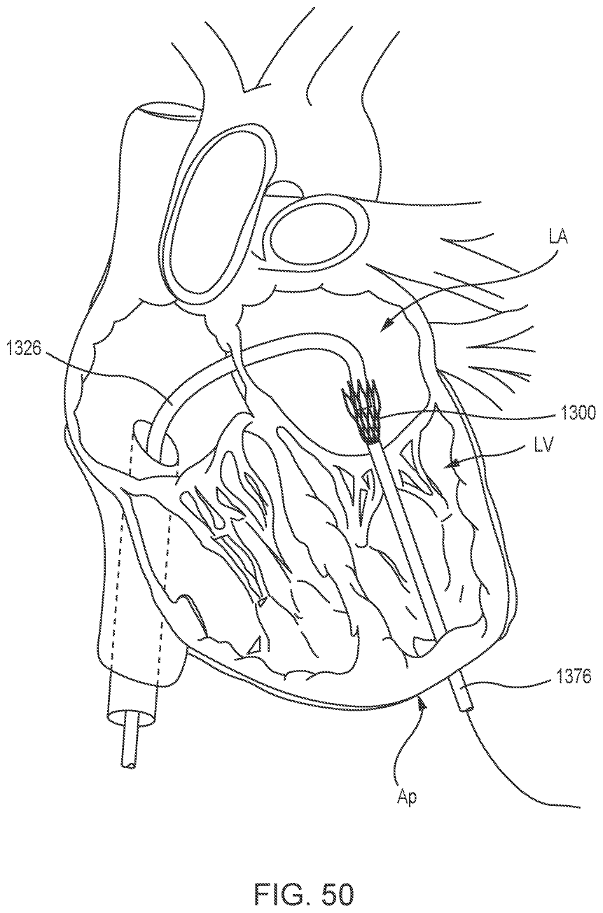

As the prosthetic valve 1300 exits the lumen of the delivery sheath 1326, the outer frame assembly of the prosthetic valve 1300 exits the delivery sheath 1326 in its inverted configuration and begins to revert or flip to its expanded or deployed configuration as described above, for example, with respect to the embodiment of FIGS. 22-24, and as shown in FIG. 50. In some embodiments, the pusher device and/or the tether 1336 can be used to aid in the reversion of the outer frame assembly. As shown in FIG. 50, with the outer frame reverted and in its expanded or deployed configuration, the stopper tube 1376 and tether 1336 are manipulated or moved such that they are in a fixed position relative to the heart, with the tether 1336 pulled taut to help position and control the movement of the prosthetic valve 1300. The delivery sheath 1326 can also be moved forward or further within the left atrium LA to tilt the prosthetic valve 1300 toward the annulus such that the prosthetic valve 1300, the stopper tube 1376 and the annulus are concentric.

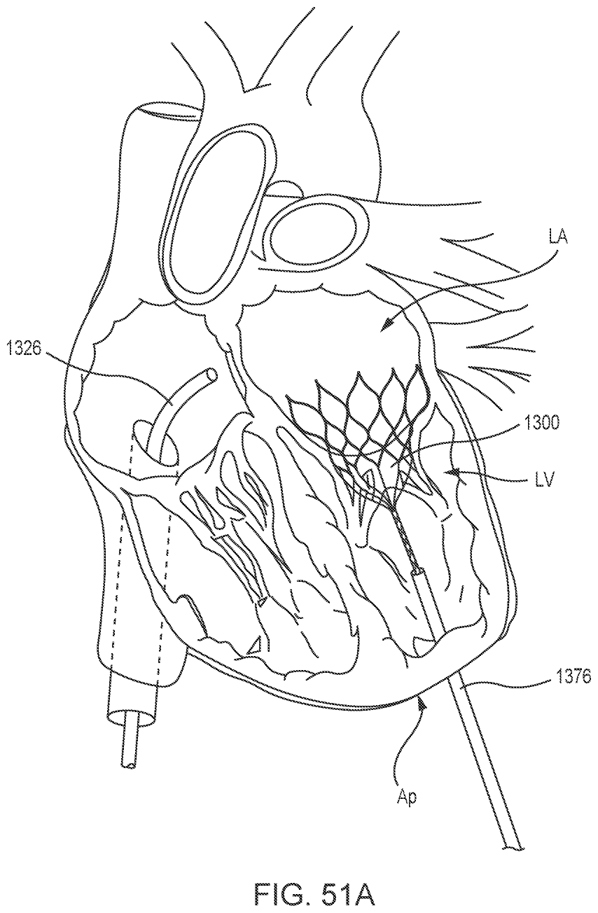

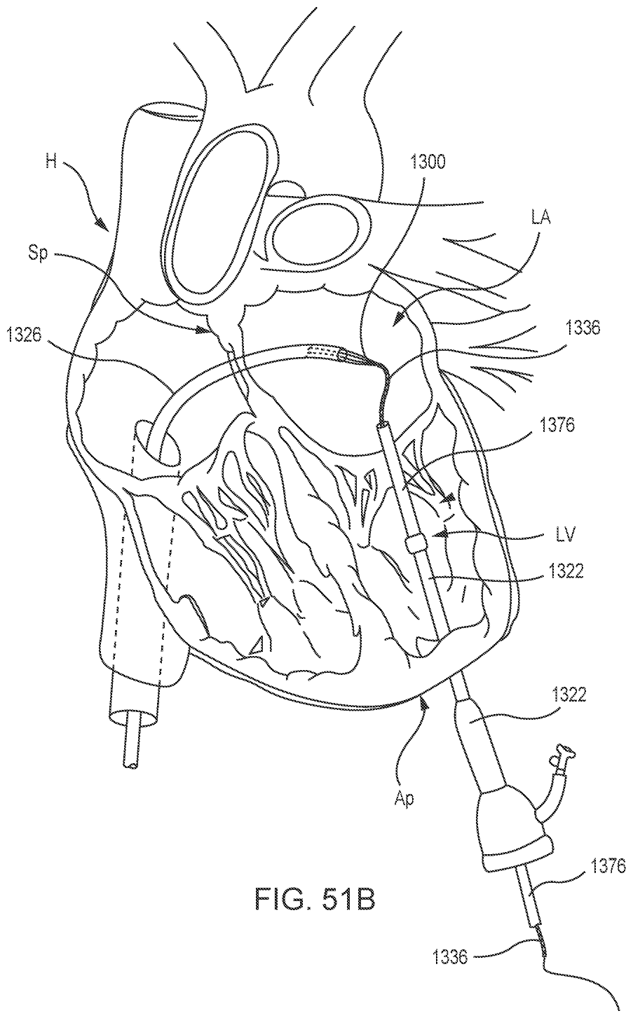

With the prosthetic valve 1300, the stopper tube 1376 and the annulus in this concentric alignment, the prosthetic valve 1300 can be pulled toward the stopper tube 1376 using the tether 1336 extending outside of a proximal end of the stopper tube 1376. The stopper tube 1376 enables the prosthetic valve to be moved/positioned in a slow and controllable manner into the annulus, as shown in FIG. 50. For example, during the deployment of the prosthetic valve 1300, the stopper tube 1376 can help rotate the prosthetic valve 1300 to achieve a desired anatomical orientation. The stopper tube 1376 can also be used to push the prosthetic valve 1300 distally or toward the annulus in the case of the prosthetic valve 1300 being deployed too low. Thus, the stopper tube 1376 can be used to move the prosthetic heart valve to a desired position within a native mitral annulus of the heart by rotating the prosthetic heart valve about a central axis of the prosthetic heart valve and/or laterally moving an orientation of the prosthetic heart valve relative to the native mitral annulus. The stopper tube 1376 can also be used to move and position the prosthetic valve in a proximal-distal direction within the heart. When the prosthetic valve 1300 is in the desired position and orientation within the annulus, the stopper tube 1376 can be pulled out through the apex Ap and the delivery sheath 1326 can also be pulled back out of the heart, as shown in FIG. 51A.

FIG. 51B illustrates an alternative approach for introducing the stopper tube 1376 into the heart. Such an approach can be used in any of the embodiments of a valve positioning device or valve alignment device described herein. As shown in FIG. 51B, a procedural catheter 1322 can first be introduced into the heart through the apex. The stopper tube 1376 can then be inserted through the procedural catheter 1322 and a distal end of the stopper tube 1376 disposed in the left atrium as described above with reference to FIGS. 48-51A.

In some embodiments, a stopper tube (or valve positioning device or valve alignment device) as described herein can be used during the deployment of a prosthetic valve that does not include a tether. For example, in such an embodiment, the prosthetic valve may have a tapered nose cone or dilator tip portion at its lead end and as it is deployed into the annulus, the distal end of the stopper tube can be placed against or over the tapered nose cone and used to help guide the positioning and orientation of the prosthetic valve within the mitral annulus. In some embodiments, for use with a prosthetic valve that does not have a tether, a temporary tether can be attached to the valve such that a valve positioning device as described herein can be used to assist in the positioning of the valve within the heart. For example, a tether can be removably attached to the valve (e.g., by tying the tether to the valve or using another suitable attachment method) prior to delivery of the valve to the heart and then removed after the valve has been placed in the desired position and orientation within the heart. The temporary tether can then be removed from the valve by, for example, cutting the tether.

In some embodiments, a stopper tube can include an expandable member at its distal end that can engage such a nose cone/dilator portion on a prosthetic valve. For example, the expandable member can be expanded to have an umbrella shape where the nose cone of the prosthetic valve can be received within the concave portion of the umbrella-shape expandable member. In some embodiments, such an expandable member can be formed with a Nitinol braid material.

In some embodiments in which the prosthetic valve does not include a tether, but instead uses a guide wire to introduce and guide a prosthetic valve during deployment, a stopper tube as described herein can be used in a similar manner as used with the tether of the embodiment of FIGS. 48-51. For example, the stopper tube can be placed over the guide wire and inserted through the apex and into the left atrium as described above. The guide wire can then be used to help position and orient the prosthetic valve in conjunction with the stopper tube.

Various alternative embodiments of a valve positioning device are described herein that can be used during the deployment of a prosthetic heart valve. The various embodiments of a valve positioning device can include an alignment member (i.e., stopper tube) and can be used to move and position a prosthetic heart valve within an annulus of the heart (e.g., native mitral annulus) to achieve a desired anatomical orientation and position within the annulus. For example, after a prosthetic valve has been delivered at least partially within a left atrium of a heart (via any of a variety of different delivery approaches such as, for example, transfemoral, transatrial, transjugular, or apically), a valve positioning device can be used to help rotate the prosthetic valve to achieve a desired anatomical orientation within the heart. The valve positioning devices described herein can also be used to push or move the prosthetic valve, for example, distally or toward the annulus in the case of the prosthetic valve being deployed too low (e.g., into the left ventricle in the case of a prosthetic mitral valve). Thus, the valve positioning devices can be used to move a prosthetic heart valve to a desired position and orientation within a native annulus of the heart by rotating the prosthetic heart valve about a central axis of the prosthetic heart valve and/or laterally moving an orientation of the prosthetic heart valve relative to the native annulus and/or moving the prosthetic valve in a proximal-distal direction within the heart.

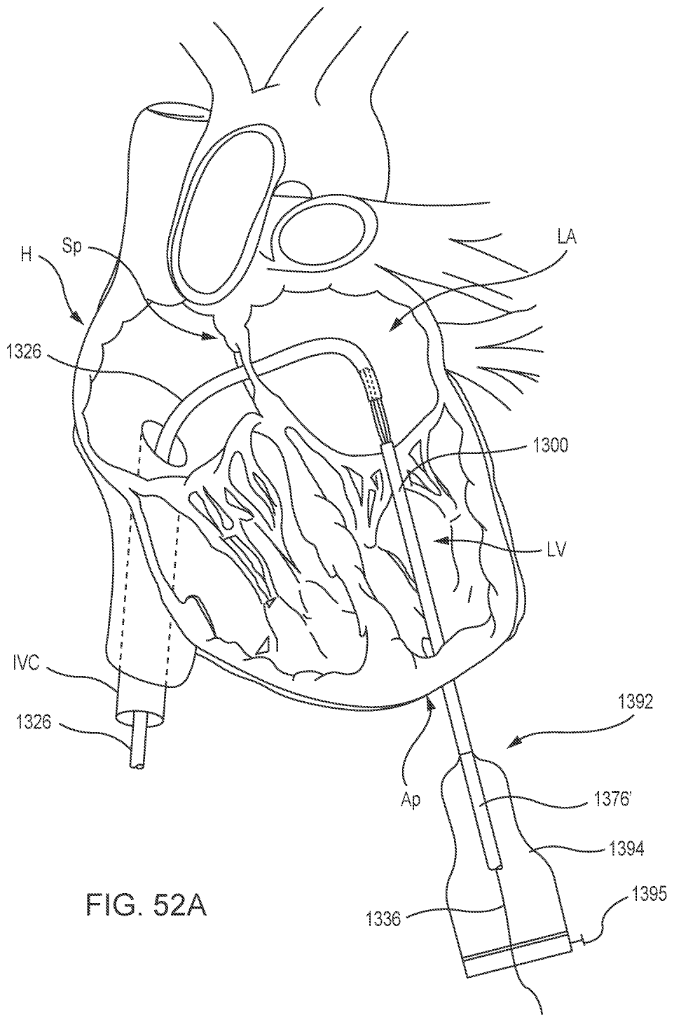

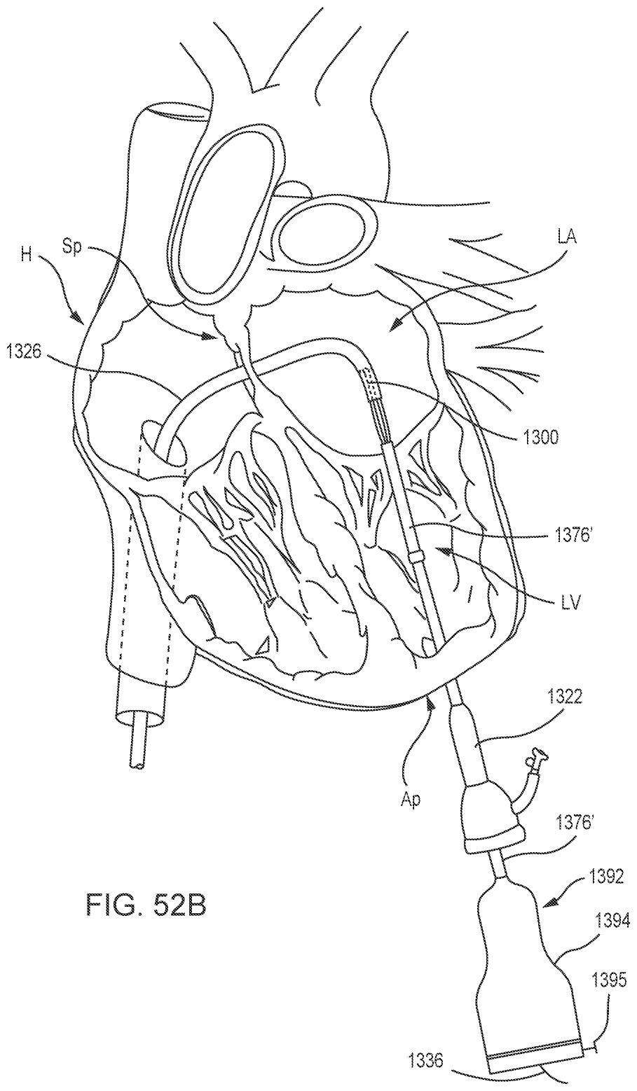

FIG. 52A illustrates another embodiment of a valve positioning device that can be used to assist in the positioning of a prosthetic valve. In this embodiment, a prosthetic valve positioning device 1392 is shown being used to position the prosthetic valve 1300 in place of the stopper tube 1376 described above. The prosthetic valve positioning device 1392 (also referred to herein as “valve positioning device” or “positioning device”) includes an alignment member 1376′ and an outer body member 1394 that has a stepped or varying outer diameter. The alignment member 1376′ can be tubular and define a lumen (not shown). The alignment member 1376′ can be the same as or similar to the stopper tube 1376. The valve positioning device 1392 can also include a tether locking device 1395. The locking member 1395 can be, for example, a pinning device used to pierce through the tether, or a vice type device configured to clamp or squeeze the tether. Other types of tether locking devices can alternatively be used. In some embodiments, the alignment member 1376′ is movably disposed within a lumen of the outer body member 1394. In some embodiments, the alignment member 1376′ is fixedly coupled to the outer body member 1394, and in yet other embodiments, the alignment member 1376′ is integrally or monolithically formed with the outer body member 1394.

As with the previous embodiment (e.g., stopper tube 1376), the alignment member 1376′ of the valve positioning device 1392 can be inserted through the apex of the heart and used to assist in the positioning of the prosthetic valve 1300 within the heart H and prevent the prosthetic valve 1300 from entering the left ventricle LV prior to delivering the prosthetic valve to the annulus of the heart. The alignment member 1376′ can be similar in structure and function to the stopper tube 1376. The outer body member 1394 has a larger outer diameter than the alignment member 1376′. The outer body member 1394 can include a distal portion having a first diameter and a proximal portion having a second diameter. The first diameter can be smaller than the second diameter to ease manipulation of the outer body member 1394 within the anatomy of a patient during use of the valve positioning device 1392. The larger diameter outer body member 1394 together with the alignment member 1376′ provides increased control and torqueability when maneuvering the valve positioning device 1392 during use. The multiple profile configuration of the valve positioning device 1392 can provide an increased amount of torque to the prosthetic valve by maximizing torque transfer through reducing the portion of the low profile portion of the device that extends outside of the apex of the heart. In other words, although a smaller diameter portion of the device is needed to be inserted into the heart, the device can include a larger profile portion of the device that extends outside of the heart that can provide strength to the device and improve torqueability.

Similar as described with reference to FIGS. 48-51 above, after the distal end of the delivery sheath 1326 is disposed within the left atrium LA of the heart H and the prosthetic valve 1300 is deployed at least partially outside of the delivery sheath 1326, the tether 1336 can be threaded or inserted through the valve positioning device 1392 as shown in FIG. 52. More specifically, the tether 1336 can be threaded or inserted through a lumen (not shown) of the alignment member 1376′, and through an opening of the locking device 1395. The alignment member 1376′ can then be inserted through the apex Ap of the heart H and a distal end of the alignment member 1376′ can be inserted through the left ventricle LV, through the annulus, and into the left atrium LA. The alignment member 1376′ at this point in the delivery procedure can be used, for example, to prevent the prosthetic valve 1300 from entering into the left ventricle LV too early or too deep.

When the prosthetic valve 1300 has partially deployed or has transitioned into its expanded or deployed configuration, the valve positioning device 1392 and the tether 1336 can be manipulated or moved to place the positioning device 1392 in engagement with the valve 1300. For example, the tether 1336 can be pulled taut while the alignment member 1376′ is moved distally such that the distal portion of the prosthetic valve 1300 (e.g., a connecting portion similar to or the same as connecting portion 544 described above with reference to FIGS. 10-12) is in contact with the distal end of the alignment member 1376′. For example, at least a portion of the connecting portion of the prosthetic valve 1300 can be disposed within a lumen of the alignment member 1376′.

With the tether 1336 held taut and the alignment member 1376′ engaged with the valve 1300, a proximal portion of the tether 1336 can be locked in place relative to the outer body member 1394 by the locking device 1395. As a result, the prosthetic valve 1300 is held in a fixed position relative to the valve positioning device 1392 and the combination of the valve positioning device 1392 and the taut tether 1336 can help position and control the movement of the prosthetic valve 1300. In other words, the alignment member 1376′ and the prosthetic valve 1300 can be moved together (e.g., rotated, moved distally/proximally, moved posteriorly/anteriorly). The delivery sheath 1326 can also be moved forward or further within the left atrium LA to tilt the prosthetic valve 1300 toward the annulus such that the prosthetic valve 1300, the alignment member 1376′ and the annulus are concentric.

During the positioning of the prosthetic valve 1300 within the heart H, the outer body member 1394 of the valve positioning device 1392 is maintained outside of the heart H and does not enter the apex Ap. In some embodiments, the alignment member 1376′ can be adjustable such that it can be extended any suitable distance from the distal end of the outer body member 1394 to accommodate a variety of heart sizes while also minimizing the extension distance of the alignment member 1376′ from the outer body member 1394. For example, in some implementations, the outer body member 1394 can be movably positioned relative to the alignment member 1376′ to adjust the length or portion of the alignment member 1376′ that extends distally from the outer body member 1394 to a desired distance within the heart. For example, the alignment member 1376′ can be movably disposed within a lumen of the outer body member 1394 and then locked at a desired position relative to the outer body member 1394. For example, in some cases, the alignment member 1376′ can be moved distally out of the lumen of the outer body member 1394 such that the alignment member 1376′ extends a distance of about 2 to about 3 cm from the distal end of the outer body member 1394 into the heart H. In some embodiments, the alignment member 1376′ is not adjustable relative to the outer body member 1394 and instead has a preset length or portion that extends from the distal end of the outer body member 1394. In some embodiments, the alignment member 1376′ and the outer body portion 1394 are formed integrally or monolithically with each other as a single component.

As a result of reducing the length of extension of the alignment member 1376′ from the outer body member 1394, the movement of the alignment member 1376′ within the heart H can be more easily controlled using the outer body member 1394. Said another way, the portion of the alignment member 1376′ extending distally from the outer body member 1394 will be more rigid the shorter distance the alignment member 1376′ extends from the outer body member 1394.

Additionally, the alignment member 1376′ can be sized to extend any suitable distance proximally within the lumen of the outer body member 1394. For example, the proximal end of the alignment member 1376′ can be positioned within or coupled to the smaller diameter distal portion of the outer body member 1394. Alternatively, for example, the proximal end of the alignment member 1376′ can extend in the larger diameter proximal portion of the outer body member 1394. In some embodiments, the alignment member 1376′ extends out a proximal end of the outer body member 1394.

With the prosthetic valve 1300, the alignment member 1376′ and the annulus in the concentric alignment shown in FIG. 52A, the valve positioning device 1392 enables the prosthetic valve 1300 to be moved/positioned in a slow and controllable manner into the annulus. For example, during the deployment of the prosthetic valve 1300, the valve positioning device 1392 can help rotate the prosthetic valve 1300 to achieve a desired anatomical orientation. The valve positioning device 1392 can also be used to push the prosthetic valve 1300 distally or toward the annulus in the case of the prosthetic valve 1300 being deployed too low. When the prosthetic valve 1300 is in the desired position and orientation within the annulus, the tether 1336 can be unlocked from the valve positioning device 1392 and the alignment member 1376′ of the valve positioning device 1392 can be pulled out through the apex Ap (e.g., along the tether 1336). The delivery sheath 1326 can also be pulled back out of the heart.

As with the previous embodiment, the valve positioning device 1392 can be introduced into the heart via the procedural catheter 1322 as shown in FIG. 52B. The procedural catheter 1322 can be inserted through the apex of the heart and into the left ventricle, and then the alignment member 1376′ can be inserted through a lumen of the procedural catheter 1322 and into the heart in a similar manner as described above for stopper tube 1376 and FIG. 51B.

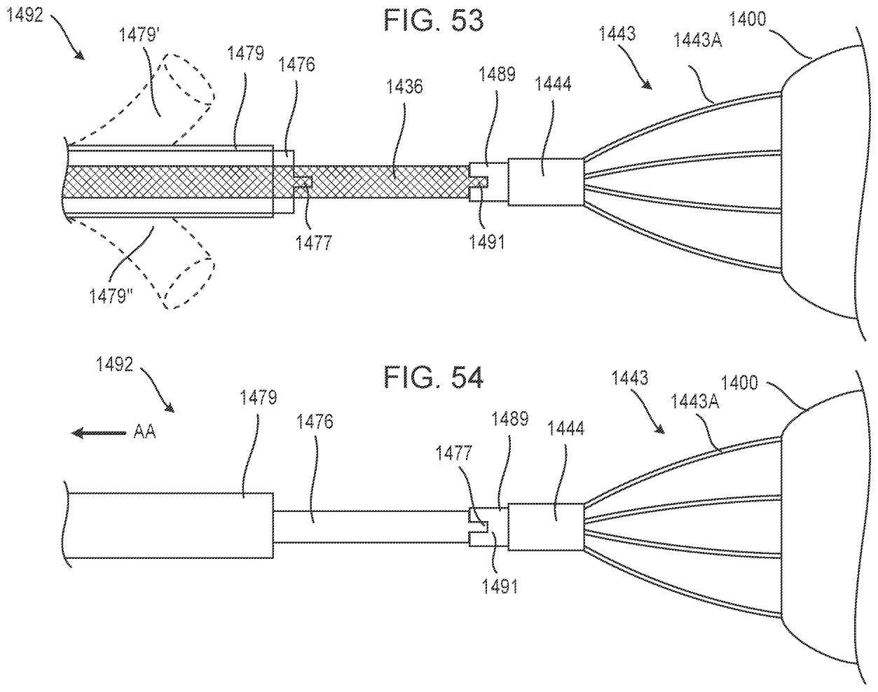

FIGS. 53 and 54 illustrate another device that can be used in positioning a prosthetic valve similarly to the stopper tube device 1376 and the valve positioning device 1392 described above. FIG. 53 is a schematic illustration of a side view of a prosthetic valve positioning device 1492 disposed in a first position and FIG. 54 illustrates the prosthetic valve positioning device 1492 disposed in a second position. The prosthetic valve positioning device 1492 (also referred to herein as “valve positioning device” or “positioning device”) includes an outer sheath 1479 and an alignment member 1476, with both the outer sheath 1479 and the alignment member 1476 shown in see-through in FIG. 53. As indicated in phantom at 1479′ and 1479″, the outer sheath 1479 can be a steerable sheath. The alignment member 1476 is movably disposed within a lumen (not shown) of the outer sheath 1479. Additionally, the alignment member 1476 includes a tether lumen (not shown) and a valve engagement feature 1477.

The valve positioning device 1492 is configured to engage with and be used to position a prosthetic valve 1400. The prosthetic valve 1400 can be substantially similar in structure and function to the prosthetic valves described herein. For example, the prosthetic valve 1400 can include an outer frame and an inner frame. The prosthetic valve 1400 can be delivered to the heart as described above for previous embodiments. For example, the prosthetic valve 1400 can be placed in the distal end of a delivery sheath and the delivery sheath can be introduced through a femoral vein puncture and extended through the inferior vena cava, into the right atrium, and then through a transseptal puncture of the septum of the heart, and into the left atrium of the heart.

Specifically, for example, the prosthetic valve 1400 includes a strut portion 1443 which includes a number of struts, such as strut 1443A. The strut portion 1443 can be substantially similar in structure and function to the strut portion 543 described above with reference to FIG. 7. The prosthetic valve 1400 also includes a connecting portion 1444. The connecting portion 1444 can be substantially similar in structure and function to the connecting portion 544 described above with reference to FIGS. 10-12. A connector 1489 extends from the connection portion 1444 and defines a recess 1491. Additionally, a tether 1436 is coupled to the connector 1489 and/or the connection portion 1444 of the valve 1400. The tether 1436 is substantially similar in structure and function to the tethers described herein. As shown in FIG. 53, the tether 1436 can extend through the lumen of the alignment member 1476.

The valve engagement feature 1477 of the alignment member 1476 and the recess 1491 of the connector 1489 are shaped and sized such that the valve engagement feature 1477 can be received within or otherwise engage with the recess 1491. In the second position shown in FIG. 54, the alignment member 1476 has been extended along the tether 1436 relative to the outer sheath 1479 into engagement with the connector 1489. In the second position, the valve engagement feature 1477 is engaged with the recess 1491 and the tether 1436 is pulled taut in the direction of arrow AA. As a result, the alignment member 1476 can be used to move/position the prosthetic valve 1400 in a slow and controllable manner into an annulus of a patient's heart. Although the valve engagement feature 1477 and the recess 1491 are shown as having complementary rectangular shapes, the valve engagement feature 1477 and the recess 1491 can have any suitable shape that allows for engagement between the alignment member 1476 and the prosthetic valve 1400. Additionally, although the valve positioning device 1492 of FIGS. 53 and 54 is shown as including only one connecting feature and one recess, the alignment member 1476 can include any suitable number of connecting features and the connector 1489 can include any suitable number of recesses. In some implementations, a connecting feature can be located on the connector 1489 and a recess can be located on the alignment member 1476.

In use, the valve positioning device 1492 of FIGS. 53 and 54 can be operated similarly to the valve positioning device (i.e., stopper tube 1376) described with reference to FIGS. 48-51. After the prosthetic valve 1400 has been at least partially deployed outside a delivery sheath in the left atrium, and the tether 1436 is extended outside of the heart, the tether 1436 can be threaded or inserted through the tether lumen of the alignment member 1476 as shown in FIG. 53. The outer sheath 1479 and the alignment member 1476 can then be inserted through the apex of the heart. The alignment member 1476 can be extended relative to the outer sheath 1479 such that a distal end of the alignment member 1476 can be inserted through the left ventricle, through the annulus, and into the left atrium. The alignment member 1476 at this point in the delivery procedure can be used, for example, to prevent the prosthetic valve 1400 from entering into the left ventricle too early or too deep.

When the prosthetic valve 1400 has partially deployed or has transitioned into its expanded or deployed configuration, the alignment member 1476 and the tether 1436 can be manipulated or moved to place the alignment member 1476 in engagement with the valve 1400. For example, the tether 1436 can be pulled taut proximally toward the operator while the alignment member 1476 is moved distally such that the recess 1491 of the connector 1489 securely engages with the connecting feature 1491 of the alignment member 1476. With the tether 1436 held taut, a proximal portion of the tether can be locked in place relative to the alignment member 1476 using a locking device (not shown). As a result, the prosthetic valve 1400 can be held in a fixed position relative to the valve positioning device 1492. Alternatively, the operator can manually maintain the tether 1436 in a taut condition while positioning the prosthetic valve 1400. With the tether 1436 pulled taut and/or locked in a fixed position relative to the alignment member 1476, the connector 1489 can remain engaged with the distal end of the alignment member 1476 during, for example, distal, proximal, lateral and/or rotational movement of the alignment member 1476 and the prosthetic valve 1400.

With the connector 1489 of the prosthetic valve 1400 and the alignment member 1476 engaged as shown in FIG. 54, the prosthetic valve 1400 can be moved/positioned within the annulus in a slow and controlled manner For example, during the deployment of the prosthetic valve 1400, the alignment member 1476 can be used to rotate the prosthetic valve 1400 to achieve a desired anatomical orientation. The alignment member 1476 can also be used to push the prosthetic valve 1400 distally or toward the annulus in the case of the prosthetic valve 1400 being deployed too low. Additionally, in embodiments where the outer sheath 1479 is steerable, the outer sheath 1479 can be manipulated to further control the movement and angular position of the prosthetic valve 1400. When the prosthetic valve 1400 is in the desired position and orientation within the annulus, the tether 1436 can be unlocked from the alignment member 1476, and the alignment member 1476 and the outer sheath 1479 can be pulled out through the apex (e.g., along the tether 1436).

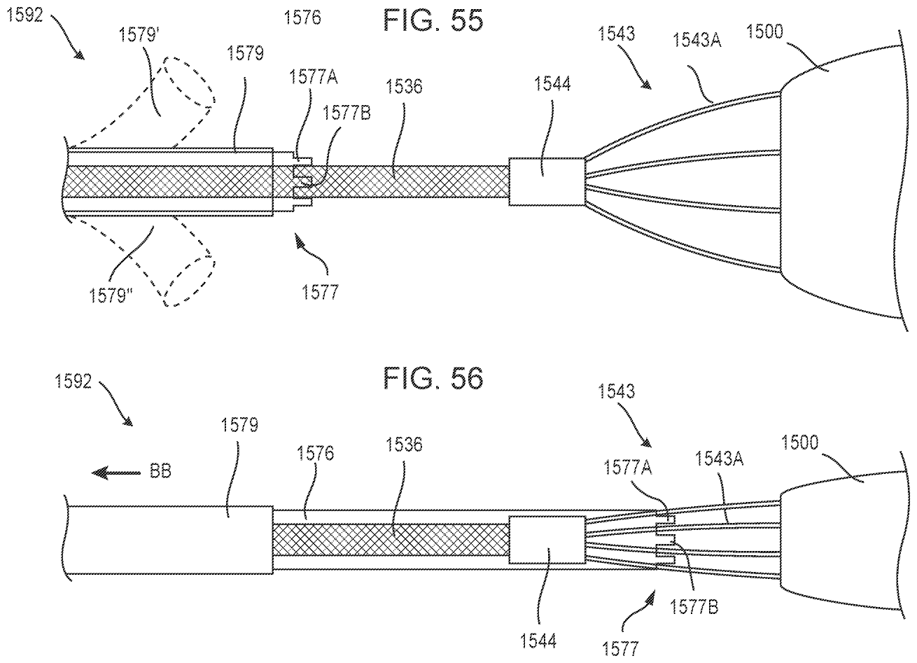

FIGS. 55 and 56 illustrate another device that can be used in positioning a prosthetic valve similarly to the stopper tube device 1376, the valve positioning device 1392, and the valve positioning device 1492 described above. FIG. 55 is a schematic illustration of a side view of a prosthetic valve positioning device 1592 shown disposed in a first position, and FIG. 56 is a side view of the prosthetic valve positioning device 1592 shown disposed in a second position. The prosthetic valve positioning device 1592 (also referred to as a “valve positioning device” or “positioning device”) includes an outer sheath 1579 and an alignment member 1576, with both the outer sheath 1579 and the alignment member 1576 shown in see-through in FIG. 55, and the alignment member 1576 shown in see-through in FIG. 56. As indicated in phantom at 1579′ and 1579″, the outer sheath 1579 can be a steerable sheath. The alignment member 1576 is movably disposed within a lumen (not shown) of the outer sheath 1579. Additionally, the alignment member 1576 includes a tether lumen (not shown) and a valve engagement portion 1577. The valve engagement portion 1577 includes multiple valve engagement features protruding from the distal end of the alignment member 1576, such as first valve engagement feature 1577A and second valve engagement feature 1577B.

The valve positioning device 1592 is configured to engage with and be used to help position a prosthetic valve 1500. The prosthetic valve 1500 can be substantially similar in structure and function to the prosthetic valves described herein. For example, the prosthetic valve 1500 can include an outer frame and an inner frame. The prosthetic valve 1500 can be delivered to the heart as described above for previous embodiments. For example, the prosthetic valve 1500 can be placed in the distal end of a delivery sheath and the delivery sheath can be introduced through a femoral vein puncture and extended through the inferior vena cava, into the right atrium, and then through a transseptal puncture of the septum of the heart, and into the left atrium of the heart.

Specifically, the prosthetic valve 1500 includes a strut portion 1543 which includes multiple struts, such as strut 1543A. The strut portion 1543 can be substantially similar in structure and function to the strut portion 543 described above with reference to FIG. 7. The prosthetic valve 1500 also includes a connecting portion 1544. The connecting portion 1544 can be substantially similar in structure and function to the connecting portion 544 described above with reference to FIGS. 10-12. Additionally, a tether 1536 is coupled to the connection portion 1544. The tether 1536 is substantially similar in structure and function to the tethers described herein. As shown in FIG. 55, the tether 1536 can extend through the lumen of the alignment member 1576.

The valve engagement portion 1577 of the alignment member 1576 is shaped and sized such that the valve engagement portion 1577 can engage with the strut portion 1543. As shown in FIG. 56, in the second position, the alignment member 1576 has been extended along the tether 1536 relative to the outer sheath 1579 into engagement with the strut portion 1543. In the second position, the valve engagement features of the valve engagement portion 1577 are engaged with the struts of the strut portion 1543 and the tether 1536 is pulled taut in the direction of arrow BB. For example, the valve engagement portion 1577 defines recesses between the valve engagement features, such as between the first valve engagement feature 1577A and the second valve engagement feature 1577B. The strut 1543A can be moved into engagement with the recess formed between the first valve engagement feature 1577A and the second valve engagement feature 1577B. With the alignment member 1576 engaged with the prosthetic valve 1500, the alignment member 1576 and the prosthetic valve 1500 can be moved together. Thus, the alignment member 1576 can be used to move/position the prosthetic valve 1500 in a slow and controllable manner into an annulus of a patient's heart. Although the valve engagement features of the valve engagement portion 1577 are shown as having rectangular shapes, the valve engagement features can have any suitable shape that allows for engagement between the alignment member 1576 and the prosthetic valve 1500. Additionally, the alignment member 1576 can include any suitable number of valve engagement features and the strut portion 1543 can include any suitable number of struts.

In use, the valve positioning device 1592 of FIGS. 55 and 56 can be operated similarly to, for example, the valve positioning device 1492 described with reference to FIGS. 53 and 54. After the prosthetic valve 1500 has been deployed outside a delivery sheath in the left atrium, the tether 1536 can be threaded or inserted through the tether lumen of the alignment member 1576 as shown in FIG. 55. The outer sheath 1579 and the alignment member 1576 can then be inserted through the apex of the heart. The alignment member 1576 can be extended relative to the outer sheath 1579 such that a distal end of the alignment member 1576 can be inserted through the left ventricle, through the annulus, and into the left atrium. Prior to engagement of the valve engagement portion 1577 with the prosthetic valve 1500, the alignment member 1576 at this point in the delivery procedure can be used, for example, to prevent the prosthetic valve 1500 from entering into the left ventricle too early or too deep as described above.

When the prosthetic valve 1500 has partially deployed or has transitioned into its expanded or deployed configuration, the alignment member 1576 and the tether 1536 can be manipulated or moved to engage the valve 1500. For example, the tether 1536 can be pulled taut proximally toward the operator while the alignment member 1576 is moved distally along the tether 1536 until the valve engagement portion 1577 securely engages with the strut portion 1543 of the alignment member 1576. With the tether 1536 held taut, a proximal portion of the tether 1536 can be locked in place relative to the alignment member 1576 using a locking device (not shown) as described above. As a result, the prosthetic valve 1500 can be held in a fixed position relative to the valve positioning device 1592. Alternatively, the operator can manually maintain the tether 1536 in a taut condition while positioning the prosthetic valve 1500. With the tether 1536 pulled taut and/or locked in a fixed position relative to the alignment member 1576, the strut portion 1543 of the valve 1500 can remain engaged with the distal end of the alignment member 1576 during, for example, distal, proximal, lateral and/or rotational movement of the alignment member 1576.

With the strut portion 1543 of the prosthetic valve 1500 and the valve engagement portion 1577 of the alignment member 1576 engaged as shown in FIG. 56, the prosthetic valve 1500 can be moved/positioned within the annulus in a slow and controlled manner For example, during the deployment of the prosthetic valve 1500, the alignment member 1576 can be used to rotate the prosthetic valve 1500 to achieve a desired anatomical orientation. The alignment member 1576 can also be used to push the prosthetic valve 1500 distally or toward the annulus in the case of the prosthetic valve 1500 being deployed too low. Additionally, in embodiments where the outer sheath 1579 is steerable, the outer sheath 1579 can be manipulated to further control the movement and angular position of the prosthetic valve 1500. When the prosthetic valve 1500 is in the desired position and orientation within the annulus, the tether 1536 can be unlocked from the alignment member 1576, and the alignment member 1576 and the outer sheath 1579 can be pulled out through the apex (e.g., along the tether 1536).

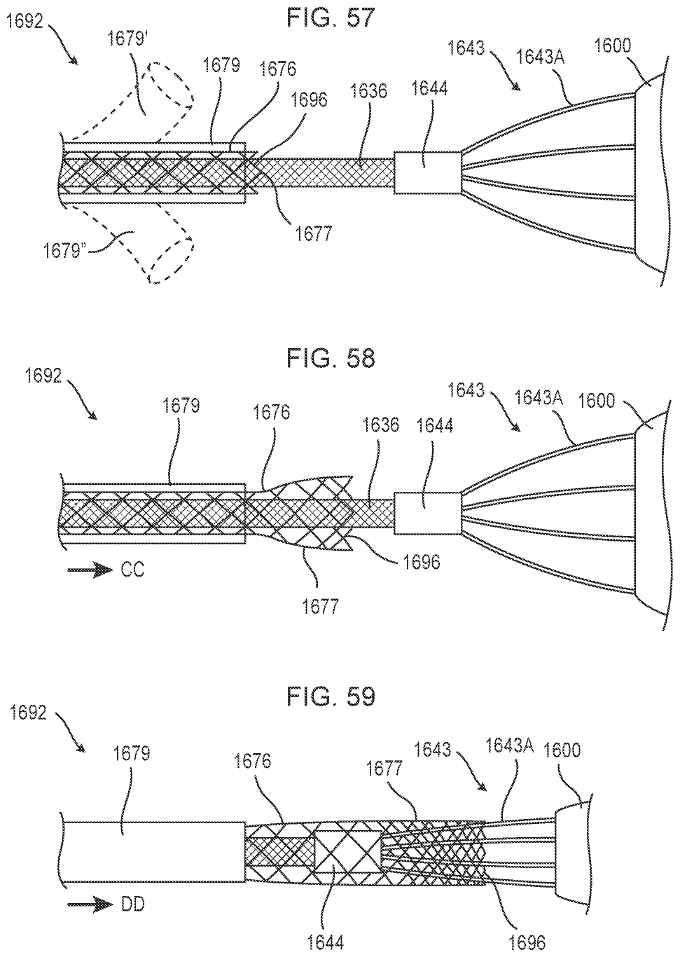

FIGS. 57-59 illustrate another device that can be used in positioning a prosthetic valve similarly to the valve positioning devices described above. FIG. 57 is a schematic illustration of a side view of a prosthetic valve positioning device 1692 disposed in a first position, FIG. 58 illustrates the prosthetic valve positioning device 1692 in a second position, and FIG. 59 illustrates the prosthetic valve positioning device 1692 in a third position. The prosthetic valve positioning device 1692 (also referred to as a “valve positioning device” or “positioning device”) includes an outer sheath 1679 and an alignment member 1676, with both the outer sheath 1679 and the alignment member 1676 shown in see-through in FIGS. 57 and 58. As indicated in phantom at 1679′ and 1679″, the outer sheath 1679 can be a steerable sheath. The alignment member 1676 is movably disposed within a lumen (not shown) of the outer sheath 1679. Additionally, the alignment member 1676 defines a distal opening 1696 in communication with a lumen (not shown) through which the tether 1636 can be inserted. The distal end of the alignment member 1676 includes a valve engagement portion 1677 described in more detail below.

The valve positioning device 1692 is configured to engage with and be used to position a prosthetic valve 1600. The prosthetic valve 1600 can be substantially similar in structure and function to the prosthetic valves described herein. For example, the prosthetic valve 1600 can include an outer frame and an inner frame. The prosthetic valve 1600 can be delivered to the heart as described above for previous embodiments. For example, the prosthetic valve 1600 can be placed in the distal end of a delivery sheath and the delivery sheath can be introduced through a femoral vein puncture and extended through the inferior vena cava, into the right atrium, and then through a transseptal puncture of the septum of the heart, and into the left atrium of the heart.

Specifically, the prosthetic valve 1600 includes a strut portion 1643 that includes multiple struts, including strut 1643A. The strut portion 1643 can be substantially similar in structure and function to the strut portion 543 described above with reference to FIG. 7. The prosthetic valve 1600 also includes a connecting portion 1644. The connecting portion 1644 can be substantially similar in structure and function to the connecting portion 544 described above with reference to FIGS. 10-12. Additionally, a tether 1636 is coupled to the connection portion 1644. The tether 1636 is substantially similar in structure and function to the tethers described herein. As shown in FIG. 57, the tether 1636 can extend through the lumen of the alignment member 1676.

The valve engagement portion 1677 is expandable from a collapsed or undeployed configuration when disposed within the outer sheath 1679, as shown in FIG. 57, to an expanded or deployed configuration when the valve positioning device 1692 is moved out of the outer sheath 1679, as shown in FIG. 58. In the second position shown in FIG. 58, the alignment member 1676 has been moved distally relative to the outer sheath 1679 in the direction of arrow CC such that the valve engagement portion 1677 extends distally from the outer sheath 1679. When the valve engagement portion 1677 is in the second position, uncompressed by the outer sheath 1679, the valve engagement portion 1677 can expand to a deployed configuration having a larger diameter than the diameter of the valve engagement portion 1677 when in the collapsed or undeployed configuration. For example, the valve engagement portion 1677 can be formed with a shape-memory material and have a biased expanded or deployed configuration such that the valve engagement portion 1677 automatically expands as it is moved out of the outer sheath 1679. In some embodiments, the valve engagement portion 1677 can be formed by laser cutting a Nitinol® tube or by braiding Nitinol® wire.

The valve engagement portion 1677 of the alignment member 1676 is shaped and sized such that, in the expanded or deployed configuration, the valve engagement portion 1677 can surround the connecting portion 1644 and engage with the strut portion 1643. For example, as the valve engagement portion 1677 expands, the distal opening 1696 becomes larger and the lumen of the alignment member 1676 that is disposed outside of the outer sheath 1679 also enlarges such that the connecting portion 1644 can be received through the distal opening 1696 and be disposed within the portion of the lumen associated with the valve engagement portion 1677 of the alignment member 1676. To move the valve engagement portion 1677 into its deployed or expanded configuration to engage with the strut portion 1643, the tether 1636 is pulled taut and the alignment member 1676 is moved distally along the tether 1636 in the direction of arrow CC relative to the outer sheath 1679, such that the engagement portion 1677 is moved outside a distal end of the outer sheath 1679. As shown in FIG. 59, after the valve engagement portion 1677 has been extended outside of the distal end of the outer sheath 1679, the engagement portion 1677 can assume its biased expanded position (as shown in FIG. 58) and can be moved further distally to surround the connecting portion 1644 and/or the strut portion 1643 of the valve 1600. The outer sheath 1679 optionally can be moved distally along the alignment member 1676 toward the prosthetic valve 1600 in the direction of arrow DD to partially collapse or compress the valve engagement portion 1677 around the connecting portion 1644 and/or strut portion 1643 of the valve 1600. Consequently, the valve engagement portion 1677 can be engaged more securely with the valve 1600. As a result, the alignment member 1676 can be used to move/position the prosthetic valve 1600 in a slow and controllable manner into an annulus of a patient's heart.

In use, the valve positioning device 1692 of FIGS. 57-59 can be operated similarly to the valve positioning device 1592 described with reference to FIGS. 55 and 56. After the prosthetic valve 1600 has been at least partially deployed outside a delivery sheath in the left atrium, and the tether 1636 is extended outside of the heart, the tether 1636 can be threaded or inserted through the lumen of the alignment member 1676 as shown in FIG. 57. The outer sheath 1679 and the alignment member 1676 can then be inserted through the apex of the heart. The alignment member 1676 can be extended relative to the outer sheath 1679 such that the valve engagement portion 1677 of the alignment member 1676 can be moved outside of the distal end of the outer sheath 1679 to assume its biased expanded configuration as described above, and be inserted through the left ventricle, through the annulus, and into the left atrium. Alternatively, the outer sheath 1679 and the alignment member 1676 can be inserted through the left ventricle, through the annulus, and into the left atrium before the valve engagement portion 1677 of the alignment member 1676 is extended and expanded relative to the outer sheath 1679. Prior to engagement of the valve engagement portion 1677 with the prosthetic valve, the outer sheath 1679 and/or the alignment member 1676 can be used, for example, to prevent the prosthetic valve 1600 from entering into the left ventricle too early or too deep as described above.

With the prosthetic valve 1600 partially deployed or in its expanded or deployed configuration, the alignment member 1676 and the tether 1636 can be manipulated or moved to engage the valve 1600. For example, the tether 1636 can be pulled taut proximally toward the operator while the alignment member 1676 is moved distally along the tether 1636 until the valve engagement portion 1677 surrounds the connecting portion 1644 and/or the strut portion 1643 of the prosthetic valve 1600 as described above. With the tether 1636 held taut, the outer sheath 1679 can then be moved distally along the tether 1636 relative to the alignment member 1676 to partially collapse or compress the valve engagement portion 1677 around the connecting portion 1644 and/or the strut portion 1643 of the valve 1600 (FIG. 59). Consequently, the valve engagement portion 1677 can be engaged more securely with the valve 1600.

In some embodiments, for even more secure engagement, with the tether 1636 held taut, a proximal portion of the tether 1636 can be locked in place relative to the alignment member 1676 using a locking device (not shown). As a result, the prosthetic valve 1600 is held in a fixed position relative to the valve positioning device 1692. Alternatively, the operator can manually maintain the tether 1636 in a taut condition while positioning the prosthetic valve 1600. With the tether 1636 pulled taut and/or locked in a fixed position relative to the alignment member 1676, the connecting portion 1644 and the strut portion 1643 of the valve 1600 can remain engaged with the distal end of the alignment member 1676 during, for example, distal, proximal, lateral and/or rotational movement of the alignment member 1676.

With the connection portion 1644 and the strut portion 1643 of the prosthetic valve 1600 and the valve engagement portion 1677 of the alignment member 1676 engaged as shown in FIG. 59, the prosthetic valve 1600 can be moved/positioned within the annulus in a slow and controllable manner For example, during the deployment of the prosthetic valve 1600, the alignment member 1676 can be used to rotate the prosthetic valve 1600 to achieve a desired anatomical orientation. The alignment member 1676 can also be used to push the prosthetic valve 1600 distally or toward the annulus in the case of the prosthetic valve 1600 being deployed too low. Additionally, in embodiments where the outer sheath 1679 is steerable, the outer sheath 1679 can be manipulated to further control the movement and angular position of the prosthetic valve 1600. When the prosthetic valve 1600 is in the desired position and orientation within the annulus, the tether 1636 can be unlocked from the alignment member 1676, and the alignment member 1676 and the outer sheath 1679 can be pulled out through the apex (e.g., along the tether 1636).

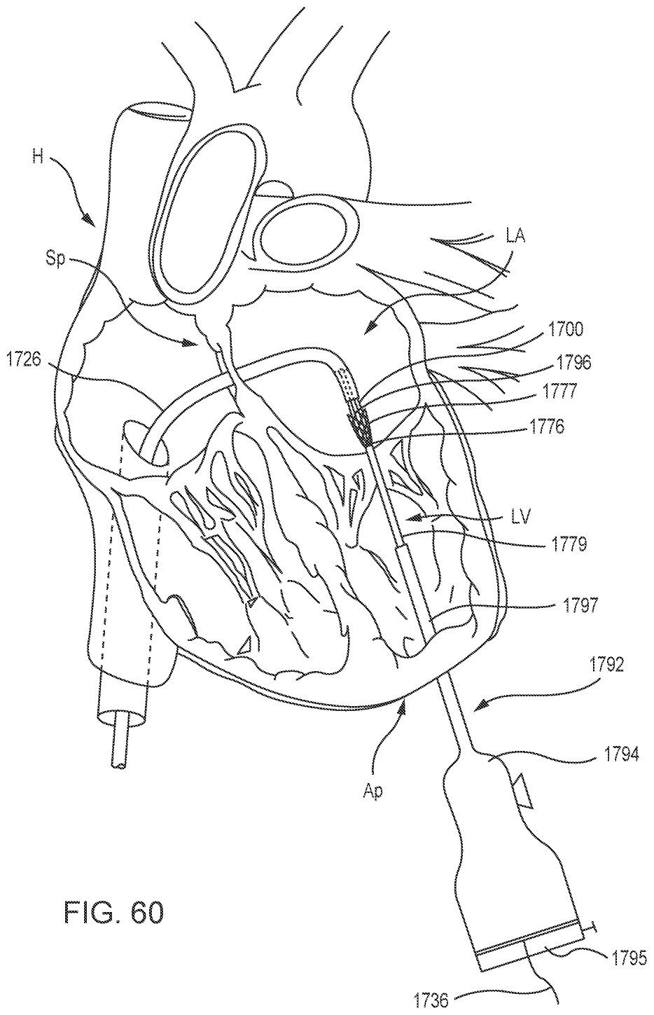

FIG. 60 illustrates another embodiment of a valve positioning device that can be used to assist in the positioning of a prosthetic valve. As shown in FIG. 60, a prosthetic valve 1700 can be constructed the same as or similar to, and function the same as or similar to, the prosthetic valves described above for previous embodiments. For example, the prosthetic valve 1700 can include an outer frame and an inner frame. Additionally, the prosthetic valve 1700 can include a connecting portion (not shown) and a strut portion (not shown) constructed the same as or similar to, and functioning the same as or similar to, the prosthetic valves described above for previous embodiments. The prosthetic valve 1700 can also include a tether 1736 that is the same as or similar to the tethers described above for previous embodiments. The prosthetic valve 1700 can be delivered to the heart H as described above for previous embodiments. For example, the prosthetic valve 1700 can be placed in the distal end of a delivery sheath 1726 and the delivery sheath 1726 can be introduced through a femoral vein puncture and extended through the inferior vena cava IVC, into the right atrium, and then through a transseptal puncture of the septum Sp of the heart H, and into the left atrium LA of the heart H.

In the embodiment of FIG. 60, a prosthetic valve positioning device 1792 is shown being used to position the prosthetic valve 1700. The prosthetic valve positioning device 1792 (also referred to herein as a “valve positioning device” or “positioning device”) includes an outer sheath 1797 extending from an outer body member 1794. The outer sheath 1797 can be tubular and defines a lumen (not shown). The outer body member 1794 can have a stepped or varying outer diameter. In some embodiments, the outer sheath 1797 is movably disposed within a lumen of the outer body member 1794. In some embodiments, the outer sheath 1797 is fixedly coupled to the outer body member 1794, and in yet other embodiments, the outer sheath 1797 is integrally or monolithically formed with the outer body member 1794. The outer body member 1794 has a larger outer diameter than the outer sheath 1797. In some embodiments, the outer body member 1794 can be the same as or similar to the outer body member 1394 described above with reference to FIG. 52. The larger diameter outer body member 1794 together with the outer sheath 1797 can provide increased control and torqueability when maneuvering the valve positioning device 1792 during use. As described previously herein, the multiple profile configuration of the valve positioning device 1792 can provide an increased amount of torque to the prosthetic valve by maximizing torque transfer through reducing the portion of the low profile portion of the device that extends outside of the apex of the heart. In other words, although a smaller diameter portion of the device is needed to be inserted into the heart, the device can include a larger profile portion of the device that extends outside of the heart that can provide strength to the device and improve torqueability.

In this embodiment, the valve positioning device 1792 also includes an inner sheath 1779 and an alignment member 1776. The inner sheath 1779 can be the same or similar in structure and function to the outer sheath 1679 described above with reference to FIGS. 57-59. For example, the inner sheath 1779 can be tubular and define a lumen (not shown). The alignment member 1776 can be constructed the same as or similar to, and function the same as or similar to, the alignment member 1676 described above. For example, the alignment member 1776 can be tubular and define a lumen (not shown) and a distal opening 1796. A distal end portion of the alignment member 1776 includes a valve engagement portion 1777. The inner sheath 1779 is movably disposable within the lumen of the outer sheath 1797. Similarly, the alignment member 1776 is movably disposable within the lumen of the inner sheath 1779. The valve positioning device 1792 can also include a locking device 1795 for locking the tether 1736 in a fixed position relative to the positioning device 1792.

As with the previous embodiment, the valve engagement portion 1777 of the alignment member 1776 can have a collapsed or undeployed configuration when disposed inside the lumen of the inner sheath 1779 and an expanded or deployed configuration when moved outside of the lumen of the inner sheath 1779. When the valve engagement portion 1777 is extended distally from the inner sheath 1779, the valve engagement portion 1777 can expand to an expanded or deployed configuration having a larger diameter than the diameter of the valve engagement portion 1777 when in a collapsed or undeployed configuration. The valve engagement portion 1777 can be formed with a shape-memory material and have a biased expanded or deployed configuration such that the valve engagement portion 1777 automatically expands as it is moved out of the inner sheath 1779. In some embodiments, the valve engagement portion 1777 can be formed by laser cutting a Nitinol® tube or by braiding Nitinol® wire. Additionally, the valve engagement portion 1777 of the alignment member 1776 is shaped and sized such that, in the expanded or deployed configuration, the valve engagement portion 1777 can surround the connecting portion of the prosthetic valve 1700 and engage with the strut portion of the prosthetic valve 1700 in the same or similar manner as described for positioning device 1692.

As with the previous embodiments, the valve positioning device 1792 can be inserted through the apex of the heart and used to assist in the positioning of the prosthetic valve 1700 within the heart H and to prevent the prosthetic valve 1700 from entering the left ventricle LV prior to delivering the prosthetic valve to the annulus of the heart. In addition, the valve positioning device 1792 can be used to position the prosthetic valve 1700 with increased control over the prosthetic valve 1700 and minimal damage to surrounding heart tissues.

Similar as described with reference to FIGS. 57-59 above, after the distal end of the delivery sheath 1726 and the prosthetic valve 1700 are disposed within the left atrium LA of the heart H, and the tether 1736 is extended outside of the heart H, the tether 1736 can be threaded or inserted through the valve positioning device 1792 as shown in FIG. 60. More specifically, the tether 1736 can be threaded or inserted through the lumen of the alignment member 1776 and through an opening of the locking device 1795. The valve positioning device 1792 can then be inserted through the apex Ap of the heart H and a distal end of the outer sheath 1797 can be inserted through the left ventricle LV, through the annulus, and into the left atrium LA. The valve positioning device 1792 at this point in the delivery procedure can be used, for example, to prevent the prosthetic valve 1700 from entering into the left ventricle LV too early or too deep as described above for previous embodiments.

When the prosthetic valve 1700 has partially deployed or has transitioned into its expanded or deployed configuration, the inner sheath 1779 can be extended distally from the outer sheath 1797 and the alignment member 1776 can be extended distally from the inner sheath 1779, simultaneously or sequentially. Similar to the alignment member 1676 described above with reference to FIGS. 57-59, as the alignment member 1776 exits the distal end of the inner sheath 1779, the valve engagement portion 1777 can expand from the undeployed or collapsed configuration into the deployed or expanded configuration. After the valve engagement portion 1777 has expanded to a diameter larger than the distal end of the prosthetic valve 1700 (e.g., the connection portion and/or the strut portion), the alignment member 1776 and the tether 1736 can be manipulated or moved to place the valve engagement member 1777 in engagement with the valve 1700. For example, the tether 1736 can be pulled taut proximally toward the operator while the alignment member 1776 is moved distally such that the valve engagement portion 1777 surrounds the connecting portion and/or the strut portion of the prosthetic valve 1700. Once the valve engagement portion 1777 is positioned such that it surrounds the connecting portion and/or portion of the strut portion, with the tether 1736 held taut, the inner sheath 1779 can be moved distally along the tether 1736 and relative to the alignment member 1776 to partially collapse or compress the valve engagement portion 1777 of the alignment member 1776 around the connecting portion and/or the strut portion of the prosthetic valve 1700. Consequently, the valve engagement portion 1777 can be engaged more securely with the valve 1700. As a result, the alignment member 1776 can be used to move/position the prosthetic valve 1700 in a slow and controllable manner into an annulus of a patient's heart. The delivery sheath 1726 can also be moved forward or further within the left atrium LA to tilt the prosthetic valve 1700 toward the annulus such that the prosthetic valve 1700 and the valve engagement portion 1777 are positioned in preparation for engagement and such that the prosthetic valve 1700, the valve engagement portion 1777 and the annulus are concentric.

In some embodiments, for ease of manipulating the valve positioning device 1792, with the tether 1736 held taut, a proximal portion of the tether 1736 can be locked in place relative to the alignment member 1776 using the locking device 1795. Alternatively, the operator can manually maintain the tether 1736 in a taut condition while positioning the valve engagement portion 1777 and the inner sheath 1779 relative to the prosthetic valve 1700. With the tether 1736 pulled taut and/or locked in a fixed position relative to the alignment member 1776, the connecting portion and a portion of the strut portion of the valve 1700 can remain within the valve engagement portion 1777 of the alignment member 1776 while the inner sheath 1779 is moved distally into compressing engagement with the valve engagement portion 1777 as described for the previous embodiment. With the inner sheath 1779 partially collapsing or compressing the valve engagement portion 1777 around the connecting portion or a portion of the strut portion of the valve 1700, and the tether 1736 maintained in a taut and/or fixed position relative to the engagement member 1777, the alignment member 1776 can be used to move the valve 1700, for example, distally, proximally, and/or rotationally. Alternatively, after the alignment member 1776 and inner sheath 1779 have engaged the valve 1700, the need to maintain the tether 1736 in a taut or fixed position relative to the alignment member 1776 may no longer exist. Similar to the outer body member 1392 described above with reference to FIG. 52, during the positioning of the prosthetic valve 1700 within the heart H, the outer body member 1794 of the valve positioning device 1792 is maintained outside of the heart H and does not enter the apex Ap.

In some embodiments, the outer sheath 1797 can be adjustable such that it can be extended any suitable distance from the distal end of the outer body member 1794 to accommodate a variety of heart sizes while also minimizing the extension distance of the outer sheath 1797 from the outer body member 1794. For example, in some implementations, the outer body member 1794 can be movably positioned relative to the outer sheath 1797 to adjust the length or portion of the outer sheath 1797 that extends distally from the outer body member 1794 to a desired distance within the heart. For example, the outer sheath 1797 can be movably disposed within a lumen of the outer body member 1794 and then locked at a desired position relative to the outer body member 1794. For example, in some cases, the outer sheath 1797 can be moved distally out of the lumen of the outer body member 1794 such that the outer sheath 1797 extends a distance of about 2 to about 3 cm from the distal end of the outer body member 1794 into the heart H.

In some embodiments, the outer sheath 1797 is not adjustable relative to the outer body member 1794 and instead has a preset length or portion that extends from the distal end of the outer body member 1794. In some embodiments, the outer sheath 1797 and the outer body portion 1794 are formed integrally or monolithically with each other as a single component. As a result of reducing the length of extension of the outer sheath 1797 from the outer body member 1794, the movement of the outer sheath 1797 within the heart H can be more easily controlled using the outer body member 1794. Said another way, the portion of the outer sheath 1797 extending distally from the outer body member 1794 will be more rigid the shorter distance the outer sheath 1797 extends from the outer body member 1794. Additionally, similar to the alignment member 1376′ described above, the outer sheath 1797 can be sized to extend any suitable distance proximally within the outer body member 1794.

Although the outer sheath 1797 is described above as being initially extended such that a distal end of the outer sheath 1797 is inserted through the left ventricle LV, through the annulus, and into the left atrium LA before the inner sheath 1779 is pushed distal of the outer sheath 1797, the distal end of the outer sheath 1797 can be positioned in any suitable position in the heart H for the extension of the inner sheath 1779 from the outer sheath 1797. For example, the distal end of the outer sheath 1797 can be positioned in the left ventricle LV or the annulus when the inner sheath 1779 is extended from the outer sheath 1797.

With the prosthetic valve 1700, the alignment member 1776, the inner sheath 1779, and the annulus in the concentric alignment shown in FIG. 60, the valve positioning device 1792 can be used to move and position the prosthetic valve 1700 within the annulus in a slow and controllable manner For example, during the deployment of the prosthetic valve 1700, the valve positioning device 1792 can help rotate the prosthetic valve 1700 to achieve a desired anatomical orientation. The valve positioning device 1792 can also be used to push the prosthetic valve 1700 distally or toward the annulus in the case of the prosthetic valve 1700 being deployed too low. When the prosthetic valve 1700 is in the desired position and orientation within the annulus, the tether 1736 can be unlocked from the locking device 1795 to allow movement of the positioning device 1792 relative to the tether 1736. The inner sheath 1779 can be pulled proximally relative to the alignment member 1776, releasing the compression on the valve engagement portion 1777 of the alignment member 1776. The alignment member 1776 can be pulled proximally relative to the inner sheath 1779 until the alignment member 1776 is in the collapsed or unexpanded position within the inner sheath 1779. The inner sheath 1779 and the outer sheath 1797 of the valve positioning device 1792 can be pulled out through the apex Ap (e.g., along the tether 1736), and the delivery sheath 1726 can also be pulled back out of the heart. In some embodiments, the inner sheath 1779 containing the alignment member 1776 is retracted into the outer sheath 1797 and/or out of the apex Ap before removing the outer sheath 1797 from the heart H. In other embodiments, the outer sheath 1797 can be removed from the heart H before removing the inner sheath 1779 containing the alignment member 1776 from the heart H.

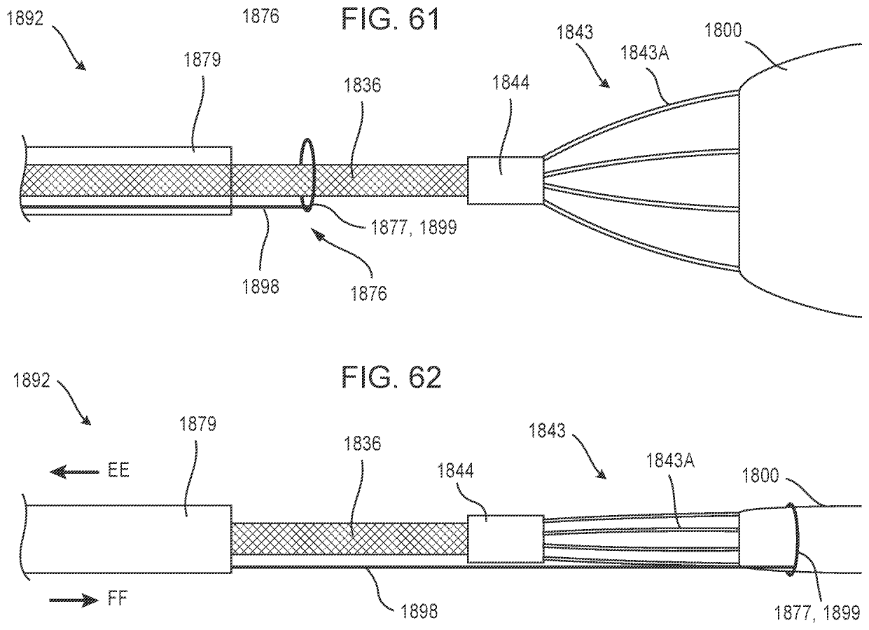

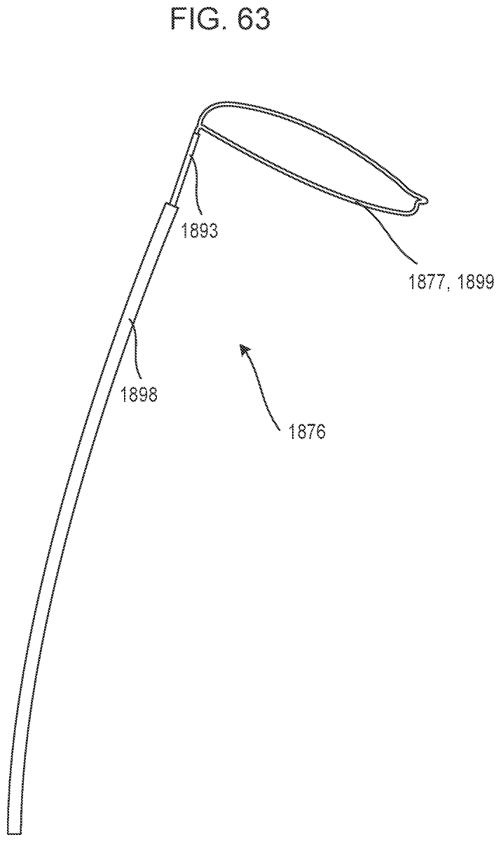

FIGS. 61 and 62 illustrate another device that can be used in positioning a prosthetic valve similarly to the valve positioning devices described above. FIG. 61 is a schematic illustration of a side view of a prosthetic valve positioning device 1892 in a first position, and FIG. 62 illustrates the prosthetic valve positioning device 1892 in a second position. The prosthetic valve positioning device 1892 (also referred to herein as “valve positioning device” or “positioning device”) includes an outer sheath 1879 and an alignment member 1876, with the outer sheath 1879 shown in see-through in FIG. 61. Similar to the outer sheaths described above, the outer sheath 1879 can be a steerable sheath. The alignment member 1876 is movably disposed within a lumen (not shown) of the outer sheath 1879. In this embodiment, the alignment member 1876 includes an elongated member 1898 and a valve engagement portion 1877 extending from the distal end of the elongated member 1898. The valve engagement portion 1877 includes a snare portion 1899 shaped as a loop. In some embodiments, the snare portion 1899 of the valve engagement portion 1877 can have a gooseneck shape. For example, the alignment member 1876 can be an Amplatz GooseNeck® Snare or Amplatz GooseNeck® Microsnare, such as is shown in FIG. 63. In some embodiments, the elongated member 1898 is shaped as a microcatheter or a tube and includes a lumen (not shown). In such embodiments, the snare portion 1899 can be coupled to or formed with an elongated wire 1893 that is movably disposed within the lumen of the elongated member 1898 as shown in FIG. 63. In other embodiments, the elongated member 1898 can be shaped as a wire and the snare portion 1899 can be coupled directly thereto.

As with previous embodiments, the valve positioning device 1892 is configured to engage with and be used to position a prosthetic valve 1800. The prosthetic valve 1800 can be substantially similar in structure and function to the prosthetic valves described herein. For example, the prosthetic valve 1800 can include an outer frame similar in structure and function to outer frame assembly 510 described above with reference to FIG. 7 and an inner frame similar in structure and function to the inner frame 550 described above with reference to FIG. 10. The prosthetic valve 1800 can be delivered to the heart as described above for previous embodiments. For example, the prosthetic valve 1800 can be placed in the distal end of a delivery sheath and the delivery sheath can be introduced through a femoral vein puncture and extended through the inferior vena cava, into the right atrium, and then through a transseptal puncture of the septum of the heart, and into the left atrium of the heart.

Specifically, for example, the prosthetic valve 1800 includes a strut portion 1843 which includes multiple struts, such as strut 1843A. The strut portion 1843 can be substantially similar in structure and function to the strut portion 543 described above with reference to FIG. 7. The prosthetic valve 1800 also includes a connecting portion 1844 that can be substantially similar in structure and function to the connecting portion 544 described above with reference to FIGS. 10-12. Additionally, a tether 1836 is coupled to the connection portion 1844. The tether 1836 is substantially similar in structure and function to the tethers described above. In this embodiment, as shown in FIG. 57, the tether 1836 can extend through the snare portion 1899 of the valve engagement portion 1877 and through the lumen of the outer sheath 1879.

The valve engagement portion 1877 is extendable from an undeployed configuration (not shown) when disposed within the lumen of the outer sheath 1879 to a deployed configuration when disposed outside of the outer sheath 1879, as shown in FIG. 61. In some embodiments, the undeployed configuration of the valve engagement portion 1877 can be a collapsed configuration where the valve engagement portion 1877 is bent, squeezed, or otherwise reduced in diameter to fit into the lumen of the outer sheath 1879. The valve engagement portion 1877 can be formed with a shape-memory material and have a biased expanded or deployed configuration such that the valve engagement portion 1877 automatically expands as it is moved out of the lumen of the outer sheath 1879. Alternatively, the valve engagement portion 1877 can remain at substantially the same angle relative to the elongated member 1898 both within the outer sheath 1879 and outside of the outer sheath 1879 (e.g., about)90° as a result of having a smaller diameter than the lumen of the outer sheath 1879.

The valve engagement portion 1877 of the alignment member 1876 is shaped and sized such that, in the expanded or deployed configuration, the snare portion 1899 of the valve engagement portion 1877 can surround and engage with a portion of the outer frame of the prosthetic valve 1800. As shown in FIG. 62, the alignment member 1876 has been extended along the tether 1836 relative to the outer sheath 1879 into engagement with the valve 1800. In this position, the snare portion 1899 of the valve engagement portion 1877 is engaged with the valve 1800 and the tether 1836 is pulled taut in the direction of arrow EE. As shown in FIG. 62, the movement of the valve engagement portion 1877 in the direction of arrow FF into engagement with the prosthetic valve 1800 causes the prosthetic valve 1800 to compress. The biased outward force of the prosthetic valve 1800 on the valve engagement portion 1877 (i.e., snare portion 1899) results in a secure engagement between the prosthetic valve 1800 and the valve engagement portion 1877. As a result, the alignment member 1876 can be used to move/position the prosthetic valve 1800 in a slow and controllable manner into an annulus of a patient's heart. Additionally, depending on the size of the snare portion 1899 of the valve engagement portion 1877, the valve engagement portion 1877 can partially collapse the prosthetic valve 1800 to various sizes, as shown in FIG. 62, to allow at least a portion of the prosthetic valve 1800 to be moved into the left ventricle for deployment below the mitral annulus.

In use, the valve positioning device 1892 of FIGS. 61-62 can be operated similarly to the valve positioning device described above. After the prosthetic valve 1800 has been at least partially deployed outside a delivery sheath in the left atrium and the tether 1836 is extended outside of the heart, the tether 1836 can be threaded or inserted through the valve engagement portion 1877 (i.e., snare portion 1899) of the alignment member 1876 and through the outer sheath 1879. With the alignment member 1876 within the outer sheath 1879, the outer sheath 1879 and the alignment member 1876 can be inserted through the apex of the heart. The alignment member 1876 can be moved distally relative to the outer sheath 1879 such that the valve engagement portion 1877 (i.e., snare portion 1899) exits the lumen of the outer sheath 1879 and can assume its deployed expanded configuration. The valve engagement portion 1877 can be inserted through the left ventricle, through the annulus, and into the left atrium. Alternatively, the outer sheath 1879 and the alignment member 1876 can be inserted through the left ventricle, through the annulus, and into the left atrium before the valve engagement portion 1877 of the alignment member 1876 is extended distally outside of the lumen of the outer sheath 1879. The outer sheath 1879 and/or the alignment member 1876 at this point in the delivery procedure can be used, for example, to prevent the prosthetic valve 1800 from entering into the left ventricle too early or too deep.

When the prosthetic valve 1800 has partially deployed or has transitioned into its expanded or deployed configuration, the alignment member 1876 and the tether 1836 can be manipulated or moved to cause the valve engagement member 1877 to engage with the valve 1800. For example, the tether 1836 can be pulled taut proximally toward the operator while the alignment member 1876 is moved distally such that the valve engagement portion 1877 (i.e., snare portion 1899) compresses and securely engages with the prosthetic valve 1800.

In some embodiments, for even more secure engagement, with the tether 1836 held taut, a proximal portion of the tether 1836 can be locked in place relative to the alignment member 1876 using a locking device (not shown). Alternatively, the operator can manually maintain the tether 1836 in a taut condition while positioning the prosthetic valve 1800. With the tether 1836 pulled taut and/or locked in a fixed position relative to the alignment member 1876, and the alignment member engaged with or coupled to the outer frame of the prosthetic valve 1800, the alignment member 1876 can be used to move the valve 1800, for example, distally, proximally, and/or rotationally to position the valve 1800 in a desired position within the annulus.

In some embodiments, the snare portion 1899 of the valve engagement portion 1877 can have an adjustable diameter in order to adjust the tightness of the valve engagement portion 1877 around the valve 1800, or to accommodate different sizes of the valve 1800. For example, the alignment member 1876 can be structured so that at least a portion of the valve engagement portion 1877 can be withdrawn into a tube of the alignment member 1876 in a manner similar to a slip knot or lasso.

With the prosthetic valve 1800 and the valve engagement portion 1877 of the alignment member 1876 engaged as shown in FIG. 62, the prosthetic valve 1800 can be moved/positioned within the annulus in a slow and controllable manner For example, during the deployment of the prosthetic valve 1800, the alignment member 1876 can be used to rotate the prosthetic valve 1800 to achieve a desired anatomical orientation. The alignment member 1876 can also be used to push the prosthetic valve 1800 distally or toward the annulus in the case of the prosthetic valve 1800 being deployed too low. Additionally, in embodiments where the outer sheath 1879 is steerable, the outer sheath 1879 can be manipulated to further control the movement and angular position of the prosthetic valve 1800. When the prosthetic valve 1800 is in the desired position and orientation within the annulus, the tether 1836 can be unlocked from the alignment member 1876, and the alignment member 1876 and the outer sheath 1879 can be pulled out through the apex (e.g., along the tether 1836).

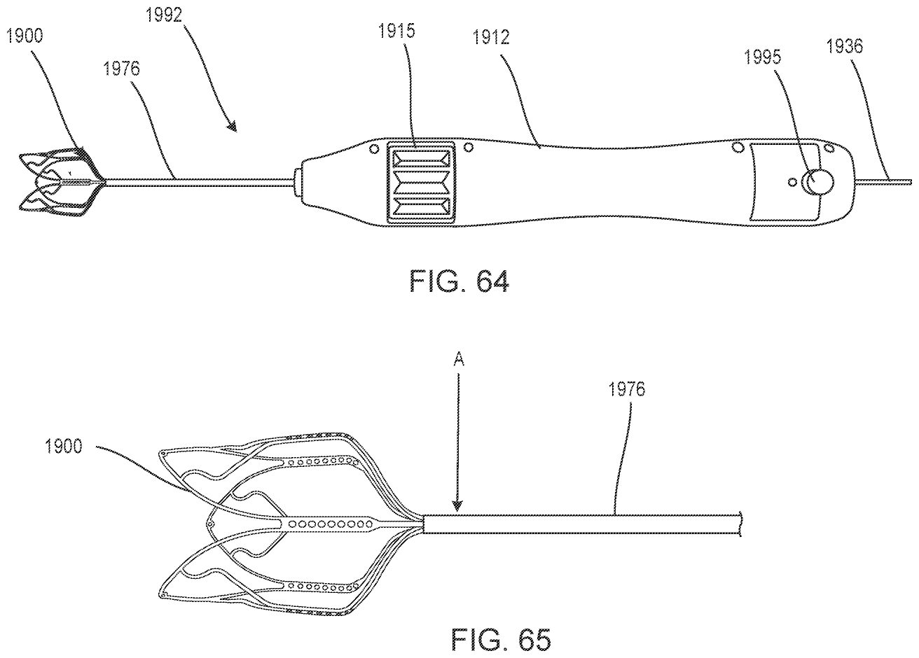

FIGS. 64-68 illustrate another device that can be used in positioning a prosthetic valve similarly to the valve positioning devices described above. A prosthetic valve positioning device 1992 (also referred to herein as “valve positioning device” or “positioning device”) includes an alignment member 1976 operatively coupled to a handle assembly 1911. The alignment member 1976 can be tubular and define a lumen (not shown) and can be configured the same as or similar to the stopper tube 1376 described above. The valve positioning device 1992 includes a tether locking device 1995 and an actuator 1915 disposed on the handle assembly 1911. The locking member 1995 can be, for example, a pinning device used to pierce through a tether 1936 extending from a prosthetic valve 1900 as previously described. In alternative embodiments, the locking member can be a vice-type device configured to clamp or squeeze the tether. Other types of tether locking devices can alternatively be used. The alignment member 1976 is movably and operatively coupled to the actuator 1915 such that when the actuator is actuated, the alignment member 1976 can be moved longitudinally relative to the handle assembly 1911. In other words, during use the actuator 1915 can be used to move the alignment member 1976 proximally or distally as described in more detail below.

As with the previous embodiments, the alignment member 1976 of the valve positioning device 1992 can be inserted through the apex of the heart and used to assist in the positioning of a prosthetic valve 1900 within the heart. For example, the valve positioning device 1992 can be used to prevent the prosthetic valve 1900 from entering the left ventricle prior to delivering the prosthetic valve 1900 to the annulus of the heart. The valve positioning device 1992 can also be used to rotate and orient the prosthetic valve 1900 in a desired position within the native annulus of the mitral valve. In some embodiments, the alignment member 1976 can be introduced into the heart through a procedural catheter (not shown in FIGS. 64-68) as described above, for example, with reference to FIG. 51B.

As previously described, after the prosthetic valve 1900 has been delivered to a left atrium of a heart and reverts back to a non-inverted expanded configuration, the tether 1936 can be threaded or inserted through the valve positioning device 1992 as shown in FIG. 64. More specifically, the tether 1936 can be threaded or inserted through a lumen (not shown) of the alignment member 1976, and through an interior of the handle assembly 1911 and the locking device 1995. The alignment member 1976 can then be inserted through the apex of the heart (or through a procedural catheter inserted through the apex of the heart) and a distal end of the alignment member 1976 can be inserted through the left ventricle, through the annulus, and into the left atrium. The alignment member 1976 at this point in the delivery procedure can be used, for example, to prevent the prosthetic valve 1900 from entering into the left ventricle too early or too deeply.

When the prosthetic valve 1900 has partially deployed or has transitioned into its expanded or deployed configuration, the valve positioning device 1992 can be used to help position the prosthetic valve 1900 within the annulus. More specifically, the tether 1936 can be pulled taut and then pinned to the handle assembly 1911 with the locking device 1995. With the tether 1936 pinned to the handle assembly 1911, the tether 1936 and prosthetic valve 1900 will not be able to move relative to the handle assembly 1911. In this embodiment, to move the alignment member 1976 distally, the user (e.g., physician) can actuate the actuator 1915 to cause the alignment member 1976 to move distally. In this embodiment, the actuator 1915 includes a rotatable advancement knob as shown in FIG. 64. Thus, the user rotates the advancement knob to move the alignment member 1976 distally such that the distal portion of the prosthetic valve 1900 (e.g., a connecting portion similar to or the same as connecting portion 544 described above with reference to FIGS. 10-12) is in contact with the distal end of the alignment member 1976 as shown in FIGS. 64 and 65.

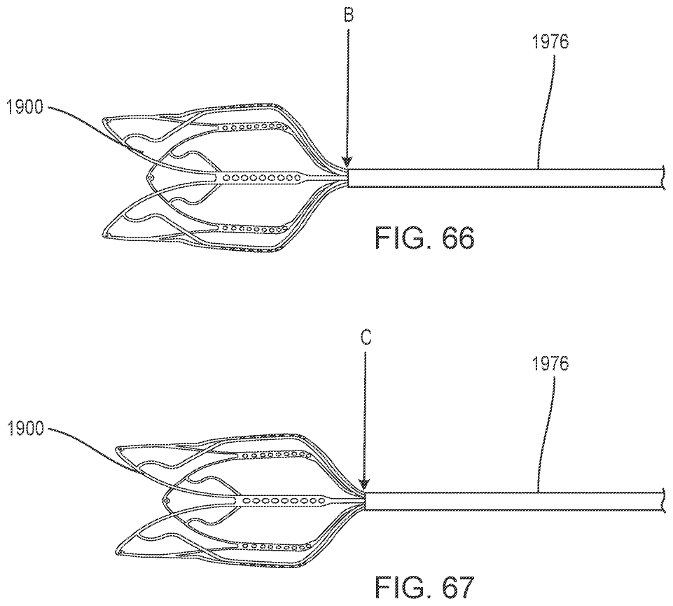

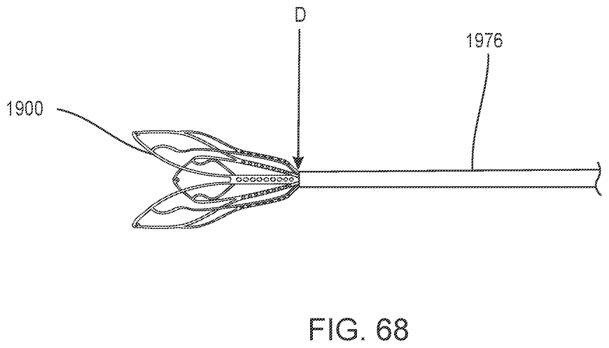

By continuing to turn the advancement knob (i.e., actuator 1915), the alignment member 1976 is advanced distally over the prosthetic valve as shown in the progression of the alignment member 1976 in FIGS. 65-68. In FIG. 65, the distal end of the alignment member 1976 is at a location A relative to the valve 1900; in FIG. 66, the distal end of the alignment member 1976 is at a location B relative to the valve 1900; in FIG. 67, the distal end of the alignment member 1976 is at a location C relative to the valve 1900; and in FIG. 68, the alignment member 1976 is at a location D relative to the valve 1900, where D is distal of C, C is distal of B and B is distal of A. As stated above, due to the tether 1936 being pinned or locked to the handle assembly 1911 and the tether 1900 being attached to the valve 1900, as the alignment member 1976 moves distally, the valve 1900 remains in a fixed position relative to the handle assembly 1911. As shown in FIG. 68, the alignment member 1976 can continue to advance distally over the micro-Vs of the connecting portion of the valve 1900, partially collapsing the valve 1900.

The valve positioning device 1992 can then be used to move and position the prosthetic valve 1900 within the annulus of the heart. For example, the alignment member 1976 and the prosthetic valve 1900 can be moved together (e.g., rotated, moved distally/proximally, moved posteriorly/anteriorly). The delivery sheath (not shown in FIGS. 64-68) used to deliver the prosthetic valve 1900 to the left atrium of the heart, can also be moved forward or further within the left atrium to help tilt the prosthetic valve 1900 toward the annulus, to for example, place the prosthetic valve 1900, the alignment member 1976 and the annulus in a concentric position relative to each other. With the prosthetic valve 1900, the alignment member 1900 and the annulus in a concentric alignment, the valve positioning device 1992 can be used to move and position the prosthetic valve 1900 with the annulus in a slow and controllable manner When the prosthetic valve 1900 is in the desired position and orientation within the annulus, the tether 1936 can be unlocked from the valve positioning device 1992 and the alignment member 1976 of the valve positioning device 1992 can be pulled out through the apex (or out through the procedural catheter).

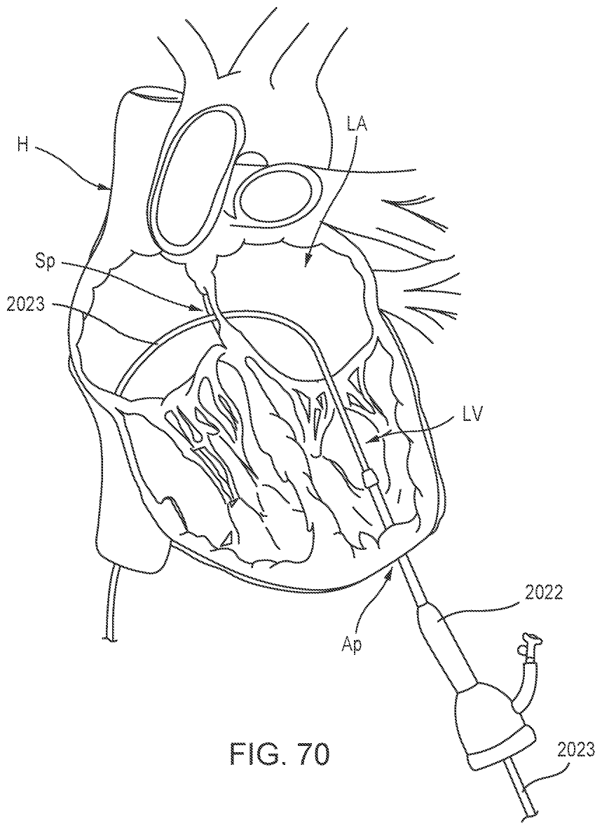

FIGS. 69-74 illustrate an alternative method of delivering a prosthetic valve within an annulus of a heart via a transfemoral delivery approach. As shown in FIG. 69, a procedural catheter 2022 is inserted through an apical puncture (e.g., a 5F apical puncture) in a ventricular wall at the apex Ap of the heart H. A guide wire 2023 is inserted through a lumen (not shown) of the procedural catheter 2022 and extended through the left ventricle LV, through a mitral valve gap and into the left atrium LA. A delivery sheath 2026 is introduced through a femoral vein puncture and extended through the inferior vena cava, into the right atrium, and then through a transseptal puncture of the septum Sp of the heart H, and into the left atrium LA of the heart H. A snare device 2028 is movably disposed within the delivery sheath 2026 and used to grab or snare a distal end portion of the guide wire 2023, as shown in FIG. 69. The snare device 2028 can be used to pull the guide wire 2023 through the delivery sheath 2026 such that the distal end portion of the guide wire 2023 extends outside the femoral vein and a proximal end of the guide wire 2023 is disposed through the ventricular wall at the apex Ap of the heart H, as shown in FIG. 70. Although not shown in FIGS. 69 and 70, the procedural catheter 2022 is disposed outside the patient's body, the distal end of the guide wire 2023 extends outside the femoral vein and outside the patient's body, and the proximal end of the guide wire 2023 extends outside the apex Ap and outside the patient's body. Although the above described snare process describes delivering the guide wire 2023 to the left atrium of the heart and then snaring the guide wire 2023 using the snare device 2028, in alternative embodiments, the guide wire 2023 can be delivered to the left ventricle LV and the snare device 2028 and delivery sheath 2026 can be inserted through the mitral annulus and into the left ventricle LV to grab or snare the guide wire 2023 as described above.

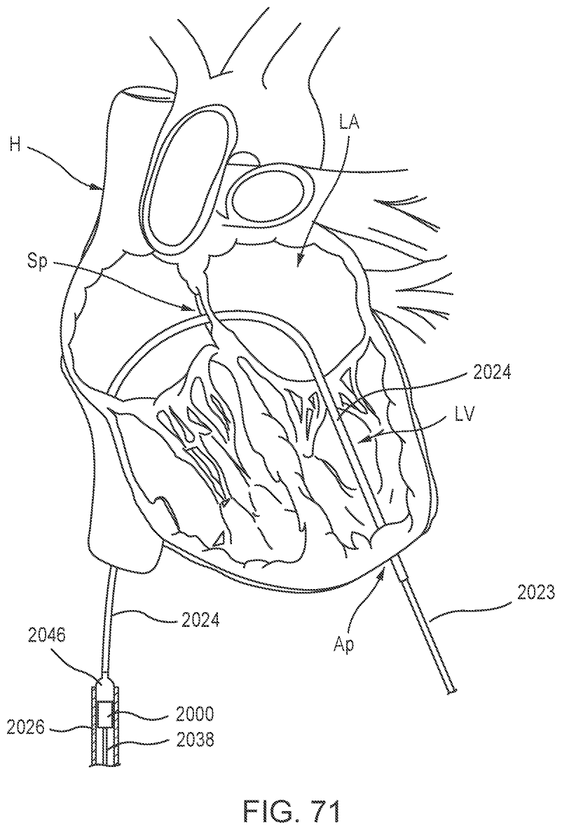

After the guide wire 2023 has been extended between the apex Ap and the access site to the femoral vein, the delivery sheath 2026 can be removed. A leader tube 2024 is loaded over the guide wire 2023 starting outside the heart (and outside the procedural catheter 2022) and exiting the femoral vein at the femoral puncture site as shown in FIG. 71. As shown in FIG. 71, the leader tube 2024 includes a balloon dilator member 2046 that is inserted into a distal end of the delivery sheath 2026 and disposed partially over a distal end portion of the prosthetic valve 2000. For example, the balloon dilator member 2046 can have a collapsed or uninflated configuration (not shown) for delivery over the guide wire 2023 and can then be inflated or otherwise moved to an expanded configuration as shown in FIG. 71. Also shown in FIG. 71, a pusher 2038 is disposed within the lumen of the delivery sheath 2026 and can be used to move or push the prosthetic valve into the left atrium LA, as described in more detail below. With the leader tube 2024 disposed between the femoral puncture site and the apex of the heart, the guide wire 2023 can be removed. Although not shown in FIGS. 71-73, the procedural catheter 2022 remains inserted into the left ventricle LV of the heart as shown in FIGS. 69 and 70.

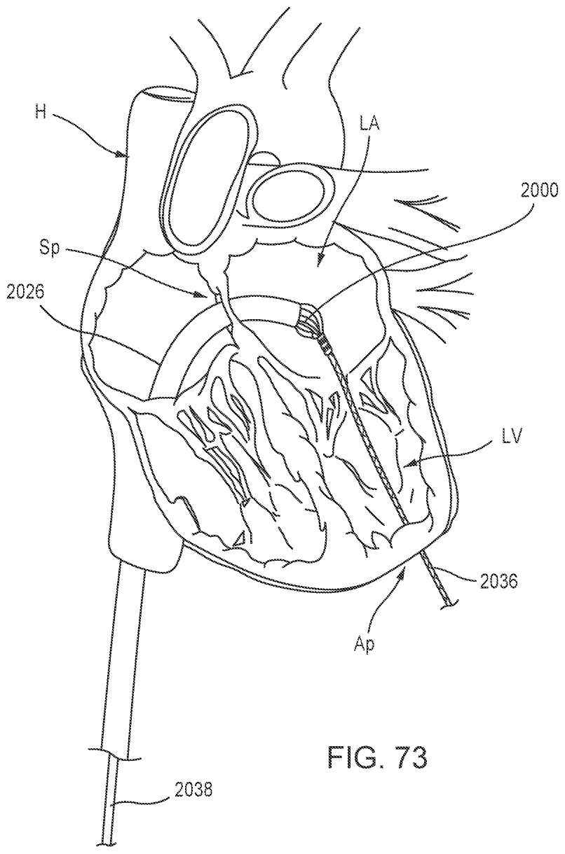

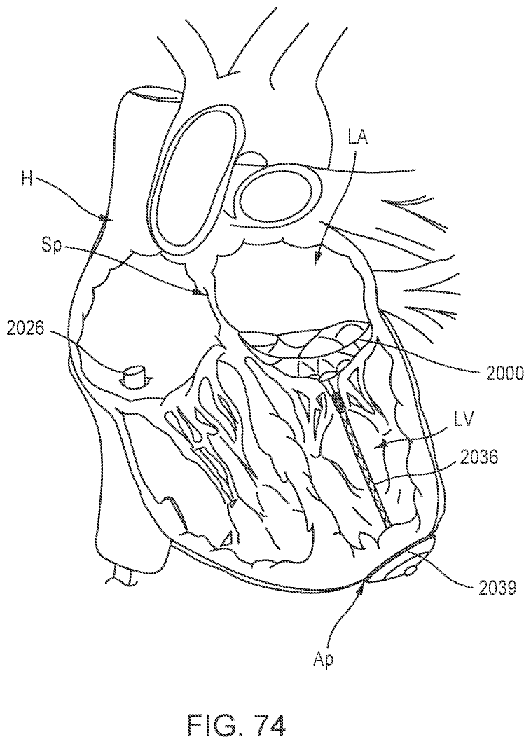

The prosthetic valve 2000 can be configured the same as or similar to the prosthetic valves described herein. The prosthetic valve 2000 (shown schematically within the delivery sheath 2026 in FIG. 71) can be disposed in an inverted configuration within the delivery sheath 2026 to reduce the overall outer perimeter of the prosthetic valve 2000. A tether 2036 is coupled to a distal end portion of the prosthetic valve 2000 (see FIGS. 73 and 74). The tether 2036 can be threaded through the leader tube 2024 prior to the leader tube 2024 being disposed within the distal end of the delivery sheath 2026. For example, as previously described, the tether 2036 can include a valve leader member (not shown) similar to the valve leader member 234 described above (see, e.g., FIG. 26). The valve leader member can have a tapered distal end to aid in the insertion and maneuvering of the valve leader member through the leader tube 2024. The valve leader member can be attached at a proximal end portion of the tether 2036, which is attached to the valve 2000. The tether 2036 can be formed, for example, as a braided rope or cord. The tether 2036 can be threaded through the leader tube 2024 with the valve leader member extended out the apex of the proximal end of the leader tube 2024 outside the apex of the heart. Thus, the tether 2036 extends between the apex Ap and the femoral puncture site where it is coupled to the valve 2000.

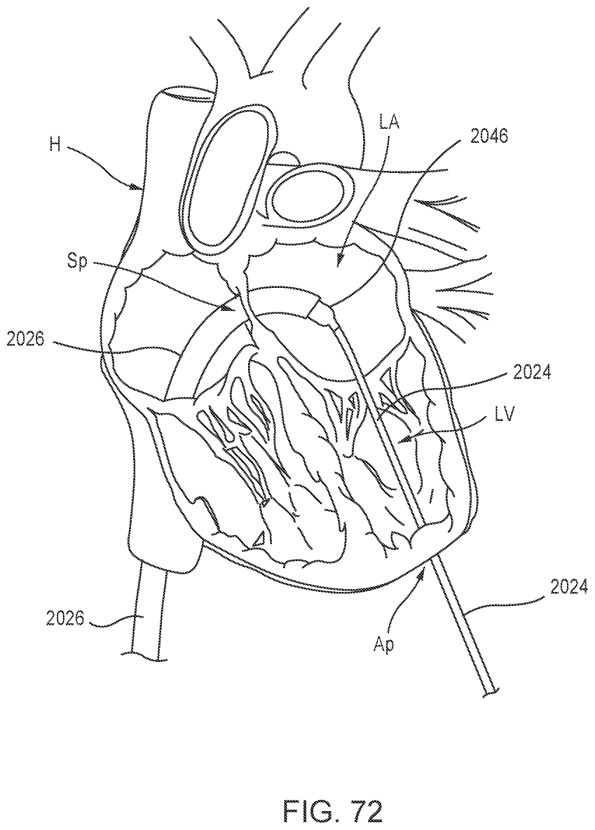

The delivery sheath 2026 can then be inserted through the femoral puncture site and moved through the femoral vein, through the inferior vena cava, into the right atrium, and then through the septum Sp until a distal end portion of the delivery sheath 2026 (with the valve 2000) is disposed within the left atrium LA, as shown in FIG. 72. The dilator balloon member 2046 can provide a smooth lead-in to assist in maneuvering the distal end of the delivery sheath 2026 through the femoral vein and within the heart. Although the delivery sheath 2026 is used to deliver both the snare device 2028 and the valve 2000, in other embodiments, a different delivery sheath can be used to deliver the snare device 2028 than is used to deliver the valve 2000.

With the distal end of the delivery sheath 2026 within the left atrium LA, the leader tube 2024 can be removed through the apex Ap, leaving the tether 2036 extended between the valve 2000 and outside the apex Ap of the heart (see FIG. 73). For example, the balloon dilator member 2046 can be moved back to a collapsed configuration for removal through the procedural catheter 2022. The procedural catheter 2022 can then also be removed. The pusher 2038 can be used to push or move the valve 2000 out the distal end of the delivery sheath 2026 and within the left atrium LA of the heart as shown in FIG. 73. As the valve exits the distal end of the delivery sheath 2026 the valve 2000 can revert and return to its original undeformed shape as described above, for example, for valve 200. For example, the valve 2000 can be formed with a shape-memory material and can have a biased undeformed shape and can be manipulated and/or deformed (e.g., compressed and/or expanded) and, when released, return to its original undeformed shape. The valve can be, for example, a valve constructed the same as or similar to, and function in the same or similar manner as, the prosthetic heart valve 500, described above.

As shown in FIG. 73, the tether 2036 extends from the valve 2000 through the apical puncture and outside the patient's body. As the delivery sheath 2026 is advanced, the tether 2036 can optionally be pulled at the apex end to help move the delivery sheath 2026, with the valve 2000 disposed therein, through the femoral vein, through the septal puncture and into the left atrium LA. The valve 2000 can then be fully deployed within the left atrium LA, as shown in FIG. 74, by using the pusher 2038 described above and/or by pulling the apex end portion of the tether 2036 until the valve 2000 is pulled out of the lumen of the delivery sheath 2026 and disposed within the left atrium LA.

In some embodiments, the pusher 2038 can also be used to aid in positioning the valve 2000 in a desired radial orientation within the left atrium LA. For example, the pusher device 2038 can define an internal lumen (not shown) that can be placed over an inner frame portion of the valve 2000 to hold the inner frame portion in a small diameter, which can help enable the valve 2000 to be positioned in a desired radial orientation and be seated within the annulus of the mitral valve. Further examples of such a valve assist device are described above with reference to FIGS. 29-31.

As shown in FIG. 74, as the valve 2000 is deployed within the left atrium LA, the valve 2000 is allowed to assume its biased expanded or deployed configuration. The delivery sheath 2026 can then be removed from the patient and the valve 2000 can be positioned and tensioned using the tether 2036 to obtain the desired or optimal location in the native mitral annulus and minimize perivalvular leaks. An epicardial pad device 2039 (as described above) can be used to secure the tether 2036 and valve 2000 in position within the mitral annulus. In some embodiments, a positioning device (not shown) can be used to help position the valve 2000 as previously described. In some embodiments, rather than securing the prosthetic mitral valve with a tether and epicardial pad, the prosthetic mitral valve can be secured with clips or other coupling methods to a portion(s) of the mitral valve apparatus and/or the ventricular wall of the heart. In some embodiments, a valve positioning device (e.g., 1376, 1392, 1492, 1592, etc.) as described above, can be used to assist in positioning the valve 2000 within the mitral annulus.

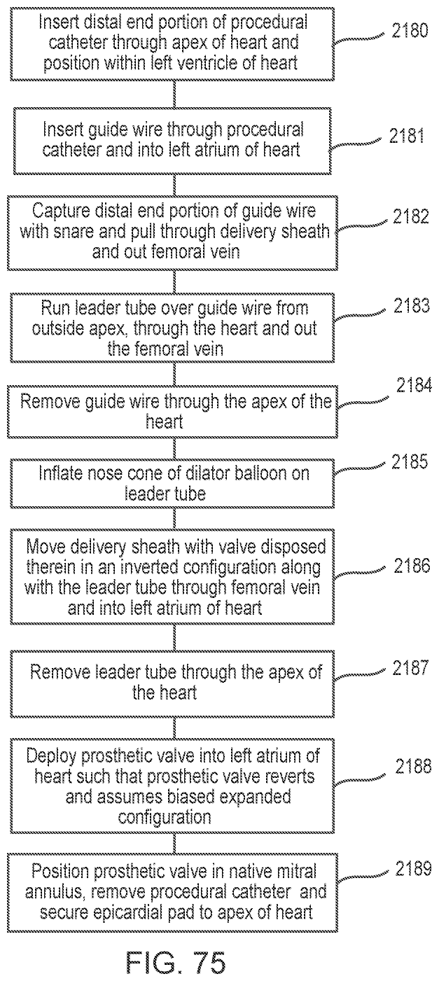

FIG. 75 is a flowchart illustrating a method of delivering and deploying a prosthetic mitral valve within a heart using a transfemoral delivery approach. The method includes at 2180, inserting a distal end portion of a procedural catheter through a puncture site at the apex of the heart, and positioning the distal end within the left ventricle of heart. At 2181, a guide wire is inserted through procedural catheter and a distal end of the guide wire is moved into the left atrium of the heart. At 2182, a distal end portion of the guide wire is captured with a snare and pulled through a delivery sheath and out the femoral vein as described above with reference to FIGS. 69-74. At 2183, a leader tube is moved or run over the guide wire from outside the apex, through the heart and out the femoral vein puncture site. At 2184, the guide wire can be removed through the apex puncture site on the heart. At 2185, the nose cone of a balloon dilator member on the leader tube can be inflated.

At 2186, the delivery sheath with a prosthetic valve disposed therein in an inverted configuration, along with the leader tube are moved through the femoral vein and into the left atrium of the heart. At 2187, the leader tube can be removed through the apex puncture site of the heart. At 2188, the prosthetic valve is deployed into the left atrium of the heart such that prosthetic valve reverts and assumes a biased expanded configuration. At 2189, the prosthetic valve is positioned within the native mitral annulus, the procedural catheter can be removed and an epicardial pad is secured to the apex of the heart.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

1. (canceled)

2. A method, comprising:
inserting a distal end of an alignment member into a left ventricle of a heart;
threading a proximal end of a tether coupled to a prosthetic heart valve through a lumen defined by the alignment member while moving the alignment member over the tether;
engaging a first portion of the prosthetic heart valve with the distal end of the alignment member; and
moving the prosthetic heart valve to a desired position within a native annulus of the heart by collectively moving the alignment member and the prosthetic heart valve.

3. The method of claim 2, further comprising:
inserting an outer sheath through an opening in an apex region of the heart prior to inserting the alignment member into the left ventricle, the alignment member being movably disposed within a lumen defined by the outer sheath.

4. The method of claim 2, further comprising:
disengaging the alignment member from the prosthetic heart valve after moving the prosthetic heart valve to the desired position; and
removing the alignment member from the heart.

5. The method of claim 2, further comprising:
capturing a second portion of the prosthetic heart valve, prior to moving the prosthetic heart valve to a desired position, by moving the alignment member distally relative to the prosthetic heart valve.

6. The method of claim 2, further comprising:
capturing a second portion of the prosthetic heart valve, prior to moving the prosthetic heart valve to a desired position, by pulling the tether proximally such that the second portion of the prosthetic heart valve is disposed within a lumen of the alignment member.

7. The method of claim 2, wherein the engaging step includes coupling the first portion of the prosthetic heart valve with an engagement region provided on the distal end of the alignment member.

8. The method of claim 7, wherein the first portion of the prosthetic heart valve comprises a plurality of struts.

9. The method of claim 8, wherein the engagement region is defined by a plurality of protrusions and a plurality of recesses, each recess being disposed circumferentially about the engagement region between adjacent protrusions and configured to receive one of the plurality of struts.

10. The method of claim 2, wherein the alignment member includes an expandable engagement region, and the engaging step includes moving the alignment member outside of an outer sheath such that the expandable engagement region moves from a collapsed configuration to an expanded configuration;
moving the expanded engagement region over the first portion of the prosthetic heart valve, and
moving the outer sheath distally to partially collapse the expandable engagement region such that the first portion of the prosthetic heart valve is captured within the expandable engagement region.

11. The method of claim 2, wherein the alignment member includes a snare including a loop, and the engaging step includes moving the snare outside of an outer sheath such that the snare moves from a closed configuration to an open configuration, and moving the snare over the tether of the prosthetic heart valve, the method further comprising:
moving the snare distally such that a first portion of the prosthetic heart valve is captured within the loop.

12. The method of claim 2, wherein the moving step includes rotating the prosthetic heart valve about a central axis of the prosthetic heart valve.

13. The method of claim 2, wherein the moving step includes laterally moving an orientation of the prosthetic heart valve relative to the native annulus.

14. The method of claim 2, further comprising:
disposing a prosthetic heart valve at least partially within a left atrium of a heart, prior to the moving step, such that a tether coupled to the prosthetic heart valve extends through the left ventricle and outside of the apex of the heart;

15. A method, comprising:
extending an engagement portion of an alignment member from an outer sheath and into a left ventricle of a heart;
inserting a proximal end of a tether coupled to a prosthetic heart valve through a lumen defined by the alignment member while moving the alignment member over the tether and toward a native annulus of the heart;
engaging a first portion of the prosthetic heart valve with the engagement portion of the alignment member;
locking the tether to a tether locking device provided on a handle assembly operatively coupled to the alignment member; and
moving the prosthetic heart valve to a desired position within the native annulus.

16. The method of claim 15, wherein the moving step comprises collectively moving the alignment member and the prosthetic valve when the first portion of the prosthetic heart valve is engaged with the alignment member.

17. The method of claim 15, wherein the moving step comprises manipulating the handle assembly to move the prosthetic heart valve.

18. The method of claim 15, wherein the moving step includes rotating the prosthetic heart valve about a central axis of the prosthetic heart valve.

19. The method of claim 15, wherein the moving step includes laterally moving an orientation of the prosthetic heart valve relative to the native annulus.

20. The method of claim 15, further comprising:
expanding the engagement portion of the alignment member from a collapsed configuration to an expanded configuration prior to the engaging step.

21. The method of claim 20, wherein the engagement portion is formed from a self-expanding material.
